SEQUENCE LISTING (354 sequences)

SEQ ID NO: 1 - DNA, 102 bp, Artificial Sequence
  Synthetic
tttaattagtacgataattaattaagttattaaatttaaaagacattattcaattaatatattatttatagatgtatagatatagctagagtcaagtttgaa

SEQ ID NO: 2 - DNA, 102 bp, Artificial Sequence
  Synthetic
ttttaggtgagaacgtcacttttatttatatagaagtatagagataatttattattcggtatatatacacgacaatatacattatgagctaattaaaatttc

SEQ ID NO: 3 - DNA, 102 bp, Artificial Sequence
  Synthetic
gtattaaatttcaaacatttaatcaaagcatatttagtacttacttacacattcactccatctcttcaaaacaaaataatcaaaaggaagttggagcctaaa

SEQ ID NO: 4 - DNA, 102 bp, Artificial Sequence
  Synthetic
attcaatccaaaaataactataaatactcattgaaatatcttctgagtcagtcttttccttaaagggaaaaataccctttttccaaaattggaaagttcatt

SEQ ID NO: 5 - DNA, 102 bp, Artificial Sequence
  Synthetic
agtgtttatatcaactcccattctgtaaatcaatccatgaatttccagcccgtgtttgaactgaacccgtcaatccacacacggggagtaatcccgtgatgg

SEQ ID NO: 6 - DNA, 102 bp, Artificial Sequence
  Synthetic
acccaatcacacagtatcatttcattttcttcttctaccttagcttcaaacgttcctcctacaacaaatcaattccaataacactataccatagctatacac

SEQ ID NO: 7 - DNA, 102 bp, Artificial Sequence
  Synthetic
tactataaccattaatgcttataactcctttgaaaacagtaccaaaagcagccacttcctagttgctcactgaacttattggttgctttctctagctcctca

SEQ ID NO: 8 - DNA, 102 bp, Artificial Sequence
  Synthetic
tctacaccaacaatgtcaaagatggtgtcttccaaaaaatttgagatcgatgctttttaatgggaagaataatttcaatttatgacagagcatggtgaaaga

SEQ ID NO: 9 - DNA, 102 bp, Artificial Sequence
  Synthetic
tggagtgcaaaactggtttctgggacatacccgatggctttaatgcgctcgtatcaagcttgcaagcaattcatatatttgttgggatcgaggatgtcgcct

SEQ ID NO: 10 - DNA, 102 bp, Artificial Sequence
  Synthetic
catgcatgccataaccagccatcaatgatgtccatgaaacaaaattcttgatgtttcatcttgttaaacacaacttgagcatcaccaatgtctccagatttg

SEQ ID NO: 11 - DNA, 102 bp, Artificial Sequence
  Synthetic
aaaaggtgtcgtggaatcatgttcattgcgaattacataggcatgaatttcgtttaccgaatcttagtgcccctaaatgagcacatgccatcaaagcacaag

SEQ ID NO: 12 - DNA, 102 bp, Artificial Sequence
  Synthetic
aaataagccaatagcatcttcaaatctaccaacatgagaatacgctgtaactcatagcattccacgataccacatccctaaccttcatttgcccaaaaactt

SEQ ID NO: 13 - DNA, 102 bp, Artificial Sequence
  Synthetic
cttcttctccttctcttacaacattgtccaactttttcactgcaacaaaaattaccatcacaaagaacccctttgaatacaacggcgaaagcgcctcttcct

SEQ ID NO: 14 - DNA, 102 bp, Artificial Sequence
  Synthetic
ccatttgtggcttcttccaactcttcataggtgaaactttgtaaattcatagccttccttgaattgggtttgtttagaaacttttgcttttctatacaagtg

SEQ ID NO: 15 - DNA, 102 bp, Artificial Sequence
  Synthetic
cttccaacactcaccatttctaaaaatagccacagcacaaaaacaatctccataaacaagccttcctgcaccaattctcatccaccaaccgaaaatactcat

SEQ ID NO: 16 - DNA, 102 bp, Artificial Sequence; identical to SEQ ID NO: 15
  Synthetic

SEQ ID NO: 17 - DNA, 102 bp, Artificial Sequence
  Synthetic
tagagactaatgttgccttttagcctttctcctccttaattagcttaaatcgataatcaaagattgcattttcaatgcaaatatatatatttattgaaactt

SEQ ID NO: 18 - DNA, 102 bp, Artificial Sequence
  Synthetic
aatgcaaatatatatatttattgaaacttgtagttattaaggagagagatgatgatgtgattgagaatggtacactatagtatacactcactccggctgtgc

SEQ ID NO: 19 - DNA, 102 bp, Artificial Sequence
  Synthetic
agacaacccaccaccaatggcgacctctcttggcgtttaaacttcgaaagtctttaaactttccaacacccacaaagagaagaaaccctcaccccctggcct

SEQ ID NO: 20 - DNA, 102 bp, Artificial Sequence
  Synthetic
attctttggtttactgctttctcaatgcaaactccaaatccgtatcaatatccctattggaaaaaggcgtatgcagccgttgggtatccttgtgtttgcgtc

SEQ ID NO: 21 - DNA, 102 bp, Artificial Sequence
  Synthetic
cgtgcctttgtccatttagattacgagttctcacacaaacccgagcatgctgcagtctcagtcttagtgttaagctgtaaaaccagttgttattatcttgtt

SEQ ID NO: 22 - DNA, 102 bp, Artificial Sequence
  Synthetic
gccaaacgagataacttagttggtttagtatttgcaatgatctggaacattcggttcgagtatgatcagtcttcaaaattttggtcagtccaaaatcaaaaa

SEQ ID NO: 23 - DNA, 102 bp, Artificial Sequence
  Synthetic
attcgagtcctttggttccaactcggtttaggaccccgaaatagaaaactgtgctaaggttccattgctcatgtactcatacaccagaagcctgtgctttct

SEQ ID NO: 24 - DNA, 102 bp, Artificial Sequence
  Synthetic
aatttccttttgcatcaaagttgatttgataatcatgaccaccaatattagaagcctattttatggtatgtaacaggagtttcaaaattgaacaaagatgtg

SEQ ID NO: 25 - DNA, 102 bp, Artificial Sequence
  Synthetic
gctttgagttttgagtgagtgatatacttactactttacaatctacaaaactagaaaaactcactatattccaaaggcatatagatacatcttatattctat

SEQ ID NO: 26 - DNA, 102 bp, Artificial Sequence
  Synthetic
cagttcaagaaatcaatgctatcaatataaaagtttttgtaatcttctaatatcatcattgcaactttgagcttcaaaaattggtatgcaccctttcatctt

SEQ ID NO: 27 - DNA, 102 bp, Artificial Sequence
  Synthetic
aacttgacacacagtttaatctaatttaaagtcaatttcttcttcattttcttattggaggggggttgggttgggtaaaaattgacttggtctataataatt

SEQ ID NO: 28 - DNA, 102 bp, Artificial Sequence
  Synthetic
gctttgagggtccaaaaagaaagcttgctaaggatccattgctcatgtactacataaacaagaagccggtgttgtccctcgttgcagaacccaattagcttt

SEQ ID NO: 29 - DNA, 102 bp, Artificial Sequence
  Synthetic
tctctcaaaactaatgagccaagtttggttagttcaacaactgagccttgagtgaacaagattgtctccattggctgaccaaacaatggttttttcgggtat

SEQ ID NO: 30 - DNA, 102 bp, Artificial Sequence
  Synthetic
caaatcataattttgaaccaaaccaatctattcttgagtggatttatttgtgtttcctttgatcattttagtttggaatttcaaaaaaccttaatggtttgg

SEQ ID NO: 31 - DNA, 102 bp, Artificial Sequence
  Synthetic
ttcggacaagctttctttcaaagcgttggaagctcatgtggtattcagttacccactctctatttccctgatttaaatttaactgccaattatgtggacaat

SEQ ID NO: 32 - DNA, 102 bp, Artificial Sequence
  Synthetic
tgtacaaggcaatcacaaatctcactctaaattgtgattggcatacgaacgaactcgtcctcagtagaatatgttatttcaaaccttcctcttcttgagaaa

SEQ ID NO: 33 - DNA, 102 bp, Artificial Sequence
  Synthetic
attcacgtgccagaaaactatgacggaaattggtttgttagtttgatgaatattttttctgaatctcaattgtccatggaacacagtaagcttgcatgttgg

SEQ ID NO: 34 - DNA, 102 bp, Artificial Sequence
  Synthetic
gtaagtaacatcaaccactctctactcttttatggatcatgggaaattaatcagaacttgcgaaagaaaagaaggaaattaatgcgtgtttgtttgtctggt

SEQ ID NO: 35 - DNA, 102 bp, Artificial Sequence
  Synthetic
atttgaaaagagttaggttaatttataatctgtcattcacttcaagagaagagatagcaattagacattaacattggtaacttatcaacaagattcgtctcc

SEQ ID NO: 36 - DNA, 102 bp, Artificial Sequence
  Synthetic
gcaattagacattaacattggtaacttatcaacaagattcgtctccctatctttcttatcttgattaatcttcttttgttattttttaattttctgaattgt

SEQ ID NO: 37 - DNA, 102 bp, Artificial Sequence
  Synthetic
cctgtaattaaagggtcagagcgttataattgtcaaattctttgtatgaactcgaaaaatattgatgatacgcccaaagatgtggcttgcaaatgatgggaa

SEQ ID NO: 38 - DNA, 102 bp, Artificial Sequence
  Synthetic
gcttctggctttttgcttcctctatctggtggagcagatagctcatctgttcgctgctatagttggctgtatgtgccagcttgttgtcaaaggtttgttttc

SEQ ID NO: 39 - DNA, 102 bp, Artificial Sequence
  Synthetic
aaattcattccttgataaataagctaaccaacactagcttcataagatagtcatattgatatttttgtctcttaattttcgtctatatggaagaataatgta

SEQ ID NO: 40 - DNA, 102 bp, Artificial Sequence
  Synthetic
gaagatggaatcgtttcgaatttccaatgaggaatacttaataaccttagcatggattagattcccaatttctttaggcaaattcaaagtagatgtggcata

SEQ ID NO: 41 - DNA, 102 bp, Artificial Sequence
  Synthetic
aatgatggctaatggcagaccagagcactttctaagcatctctaccgctaacttctttcttcctttcttcatcatcatttgagtctgaatttatatatccag

SEQ ID NO: 42 - DNA, 102 bp, Artificial Sequence
  Synthetic
aaagctccttggctaattcatcatcgcttatcattttgatttcttctctttctttctttggtgggagaagtgaaagcgaagtaaattgcttcgaagacattg

SEQ ID NO: 43 - DNA, 102 bp, Artificial Sequence
  Synthetic
tagttttgcccaaaccacccatcccacatacagagatcaccttatgcttactgagaattctctttttcagtcaaaagggcaaccaactcctcaatatcttta

SEQ ID NO: 44 - DNA, 102 bp, Artificial Sequence
  Synthetic
atccaactttgtggacattaattccctctctgaagatgttaatagatctttctgagcacaccttcattcttcttcaaagtcactttgaggacataagtctca

SEQ ID NO: 45 - DNA, 102 bp, Artificial Sequence
  Synthetic
acaaattttgattcattcaattacaacacatcaacagaagaacacaaaaaaccaaaagataggaaaaagagtcactaatgtaattattgaacaaacacacac

SEQ ID NO: 46 - DNA, 102 bp, Artificial Sequence
  Synthetic
ttaagtcaggcacaagtttcacatgagatattgaaggagccatggattaaatattcaaaccaccatactaaaaataaatggaaaaaatattacatttttcat

SEQ ID NO: 47 - DNA, 102 bp, Artificial Sequence
  Synthetic
tttattttccccacatatatataatgtgtttgtttaatatcataatctttgaattctttacatattttaatcatctatctttttatttgatatttatttaat

SEQ ID NO: 48 - DNA, 102 bp, Artificial Sequence
  Synthetic
taaacaaacacaattgattttacaagcacaattacataatcaaaagcaattgcacaacacacacattaaacatgaacataaaccagaaagttagagagacat

SEQ ID NO: 49 - DNA, 102 bp, Artificial Sequence
  Synthetic
gatttcataattaagaacccaagcaatagtcgcctagaatgtgtttctaggaattagatgaagcttgtaaatcgggtgttcttgctttactttctgttttaa

SEQ ID NO: 50 - DNA, 102 bp, Artificial Sequence
  Synthetic
taataactacatatatatattgaagatattgtttttgataattgaaaattattaattacacaataatatataagagaaaaaaaaaacaaataaagtaaattg

SEQ ID NO: 51 - DNA, 102 bp, Artificial Sequence
  Synthetic
attactcatgtcaaattatggttctttgacaccatttaggatatgtaatttaagtttaaatccaaaacaaataatataattacaaaagatagctaattgatg

SEQ ID NO: 52 - DNA, 102 bp, Artificial Sequence
  Synthetic
aattcgtaatttcttgagactctccaactgtagaagatcactcagtagaacgatccgtggtacaaatagataccaatgtttgtaaatttctagacttagttg

SEQ ID NO: 53 - DNA, 102 bp, Artificial Sequence
  Synthetic
aacactacggtattttgagtcaacgtcatgttgttgttcagcttcacctatggcagcgaattacatttgctttcagttgctcccaatcatataaggtgtgtt

SEQ ID NO: 54 - DNA, 102 bp, Artificial Sequence
  Synthetic
aaagctaaacatatatctgtttaacacactaggaacatatacaaagccaatcgaatttgacattataaatataaatggtagcaaagattaaaaaaaataaag

SEQ ID NO: 55 - DNA, 102 bp, Artificial Sequence
  Synthetic
tcgttggagcttaaaaagatggagttagaaatcgagtcaaacatagtaaactgaagctcggactatgttgactagttcaattgagaactcagttgaggatga

SEQ ID NO: 56 - DNA, 102 bp, Artificial Sequence
  Synthetic
ctttctgatgaagttcaaatgctccatcaagaggtttcgtcttcaagatcgttcccttcaggaactatctacaagaaaatctagcatccaacaggatatagc

SEQ ID NO: 57 - DNA, 102 bp, Artificial Sequence
  Synthetic
cgagaaagaaattaatgacactgttgagaaactggaggagacagaggtggctcatttcctccaaggaaaaagagttagccatggccagatttgagaggctgg

SEQ ID NO: 58 - DNA, 102 bp, Artificial Sequence
  Synthetic
aatgagacaggtaataatgtgtgcttatttaacgaaaattgttgttttaactggttttttttatttaatatttaaattaataattaggtaatattgtaatgt

SEQ ID NO: 59 - DNA, 102 bp, Artificial Sequence
  Synthetic
aacacttgcatagcttcacctaattcgccatatttaccgaatgtatcgattcatactattgtaaatttgtctgtctaatgacattcccatatccttaatctc

SEQ ID NO: 60 - DNA, 102 bp, Artificial Sequence
  Synthetic
ctttcagtattgaatttcacttcaagaccgagtcttttcacacctttgctcatgccttgagtggctcagacaacttgaaacgagactagttcttctggggat

SEQ ID NO: 61 - DNA, 102 bp, Artificial Sequence
  Synthetic
aatgagcaaaactacatcgcaccagcacatggagagcagttttaggggcaagcagagcaaaagggacataaagtatcaacttgaacatgtttagaagacaaa

SEQ ID NO: 62 - DNA, 102 bp, Artificial Sequence
  Synthetic
acccttggccaaaatcttatgaagcccgtggatttcaataatgtcatttttgagttaaagcttatcttttcttaaaaataccatttttcattctaagctttt

SEQ ID NO: 63 - DNA, 102 bp, Artificial Sequence
  Synthetic
cttagccactttctttgaatttctacttttgttgattgttcatggccatctccttctaacatctcataagcgtcattagtactctttttcatgaatgcctca

SEQ ID NO: 64 - DNA, 102 bp, Artificial Sequence
  Synthetic
gatccctgtcattgtgggtagcacatactcatgcaaacttctattttgaccaattagcctgatctactacaccccattgttatcattttgagccatatcttt

SEQ ID NO: 65 - DNA, 102 bp, Artificial Sequence
  Synthetic
gttatcattttgagccatatctttttctaactcaacatctctaatagcagtctctttccaacatcttctttcctttattctgtcgacaagcttctcaatttc

SEQ ID NO: 66 - DNA, 102 bp, Artificial Sequence
  Synthetic
gacgttgcataaactgcttgatcccgagaaaagagaaagattaacacacactagagttagtaaaaagttgcaaagaatggccctggcttggatgtgctcaac

SEQ ID NO: 67 - DNA, 102 bp, Artificial Sequence
  Synthetic
tattaagccaaaccaagttcgaatccaacctctcccaatatcgaaaaaacgaaaaaaaaaaggaaagaaaatagttgatttttttgaacaatttacatgttt

SEQ ID NO: 68 - DNA, 102 bp, Artificial Sequence
  Synthetic
agattgttggattcaaggattcttgcccgattccgttcaattttactaacgatggtgattgtccatgtattaaattatatctccaaattttaataagtacca

SEQ ID NO: 69 - DNA, 102 bp, Artificial Sequence
  Synthetic
acatcatctcattttcaaatcatacaaaacaattcaaaaacaaaaaaaatctccaacaatggcttctcactttttgaaggctgtctttgcattttgtacatt

SEQ ID NO: 70 - DNA, 102 bp, Artificial Sequence
  Synthetic
gttatttatttattgaaaatggtcatgttcaattgtgattttgtgcagatcaaagttacaaaatgtgacaaggttgtgtaaaacaaagagtattttgactgt

SEQ ID NO: 71 - DNA, 102 bp, Artificial Sequence
  Synthetic
tctaactcttattttaataaagattctaaacagagatagtactgatgaatcttggagaaaaattacagttttacaaatattgggtactatgtcgttaaaaaa

SEQ ID NO: 72 - DNA, 102 bp, Artificial Sequence
  Synthetic
gagccatttttgctataataatattttatgagatttttggttactgggatatactagagtagcataacatgagtagtgatatgagattttctacttttctag

SEQ ID NO: 73 - DNA, 102 bp, Artificial Sequence
  Synthetic
tcaaagatgctgacattgacagcgggtggtcttatacaagactgaaccccctgtcataataggtagcatataatcccccagacttctggcaggtagattctt

SEQ ID NO: 74 - DNA, 102 bp, Artificial Sequence
  Synthetic
ataaataaatcacaaaaaatactaaatgtcagattatcattacagttaaaagatactgtaattgactgaaaaagagcttgtttcaaattgtaatggtaatgg

SEQ ID NO: 75 - DNA, 102 bp, Artificial Sequence
  Synthetic
aactttttttttttgttttgttttgttatgttttgtgttttttttttcaatatacattgtctggcgtacatacattaaatttacacaatatggtgaattcaa

SEQ ID NO: 76 - DNA, 102 bp, Artificial Sequence
  Synthetic
agctttggagtggtgttacttgagattgtttgttgtagaagaaatgtggatcatggagattggtgaggaagagaaagagatcttgacagattgggcttatga

SEQ ID NO: 77 - DNA, 102 bp, Artificial Sequence
  Synthetic
attttatcactcttagtccatactctcaaagtctgcaggcatcttaaatttgccaaaagaagatggaatcttttcaatctgccaataaggaatacttaataa

SEQ ID NO: 78 - DNA, 102 bp, Artificial Sequence
  Synthetic
ttggtcataaatagcttgttaagctttttctaactaactcgtcatgtttgtccagaatcactactttgattcttgctctgctacatttttctttctcctttg

SEQ ID NO: 79 - DNA, 102 bp, Artificial Sequence
  Synthetic
atcataatcagaacttccattaacatagatggcaattcttctaacatttgctttctgttggttcttctcctttattcctccaatcaataaaatgcataaagt

SEQ ID NO: 80 - DNA, 102 bp, Artificial Sequence
  Synthetic
ggcgtgtattacattgctgagaaattgaggcccaagcatgacaatcaaagtagttccctcacatcaggatgctgatagacctttcttgcaagagtagtcttc

SEQ ID NO: 81 - DNA, 102 bp, Artificial Sequence
  Synthetic
gtttgatagatactttctcttatattttctcaatatttcatactctagactcgattgcataacaaagttgttttcaattatttatggataatacaaaaataa

SEQ ID NO: 82 - DNA, 102 bp, Artificial Sequence
  Synthetic
cgaataagacacacatagacaaactcagttgacttgacccaaagtcttcccgaagaaaatataataaatattaatttgactctgggcttctttcaatcaccc

SEQ ID NO: 83 - DNA, 102 bp, Artificial Sequence
  Synthetic
gcatacaacacttagatcatccactgtgacataaaaggaataaaagggtatcatagttctggtgtggtactgcttggaaattgtttgttgtcaaagaaactt

SEQ ID NO: 84 - DNA, 102 bp, Artificial Sequence
  Synthetic
ttgaggaatataaacgagaagatccaataaagagttgacatctttcatgatcaatgattatgaagtaactttgcattaaaatattcagtttttaataatata

SEQ ID NO: 85 - DNA, 102 bp, Artificial Sequence
  Synthetic
gataaaggaaatcaatagacaaggcagatgaagcttcttcaagctatgtccgaaaagcaaagacagttgaggcaagcttattcttatgttgaagacaatcat

SEQ ID NO: 86 - DNA, 102 bp, Artificial Sequence
  Synthetic
gcttattcttatgttgaagacaatcatgttgttggattcgataaagatattccaagagttggttgcccttttaactgaaaaagagaaccctcgtaagcataa

SEQ ID NO: 87 - DNA, 102 bp, Artificial Sequence
  Synthetic
ccttctccattcatgatctcatgagagaattgtgcgtgtctaaggctcaatgaagaaaactttctacattcaattgattgtaggaatcaatgggaagaacca

SEQ ID NO: 88 - DNA, 102 bp, Artificial Sequence
  Synthetic
atactaaatattatcgaaatatatattgaaaaactcactagagtgcaatatatatcacaataaacaactcttataccagataataagatggtgagggacatg

SEQ ID NO: 89 - DNA, 102 bp, Artificial Sequence
  Synthetic
aactcatacaccaacaaatgattcttcccatcatcacaatatccaagtagagcgaacgagattcttgtgatgtgtttggccaatggatttcaattcagcctt

SEQ ID NO: 90 - DNA, 102 bp, Artificial Sequence
  Synthetic
ccttttcgaattttgatgaaagcaatagatggacggttgtgatcaactcttcccatttgataagggaaacctcttcttccaacaagttcctcctctaagaac

SEQ ID NO: 91 - DNA, 102 bp, Artificial Sequence
  Synthetic
ggattgattagagagtacccttttaagcactcacactttggtctcttctcctttattgagtctgcaaatggtattgtatccacaaactccaattccttccca

SEQ ID NO: 92 - DNA, 102 bp, Artificial Sequence
  Synthetic
tcacaatgtatgatctgagtgttgcactcctcgtgtaaatataaaagcccagtttgcaattcctaaagcaagttcacatctttgagtccaactaggctttat

SEQ ID NO: 93 - DNA, 102 bp, Artificial Sequence
  Synthetic
tccttgtaggtaaagcaacgcaaatttgactcgggaacttcttgtggagcgaaaagaccttttattcctcttcttgtaaatgatgaaaaaccccatagacat

SEQ ID NO: 94 - DNA, 102 bp, Artificial Sequence
  Synthetic
ctttgcagccttgaatgaaatctggtttacaacttccataactgtcatttctggttgagtagagagtacccttctaagcattcacacttcggtctcttatct

SEQ ID NO: 95 - DNA, 102 bp, Artificial Sequence
  Synthetic
gtaactcctttatttgcaatcccacatggcactatgtgcttaaaatagttctaaatctggatcaatattaaatgccaacccatgagaggtgactgcatacga

SEQ ID NO: 96 - DNA, 102 bp, Artificial Sequence
  Synthetic
acctcaactaaagaatcaataagccactaaagaattactaagccactaacagttgaaatatattagacttccaaactaaatgaagagtaaataaacaaatca

SEQ ID NO: 97 - DNA, 102 bp, Artificial Sequence
  Synthetic
attgtaaaagcaacttcttttcaaacccaggaaaagaaactgggaaaaaaaggccaagaattttcttataaatatcgtggggagaaacacaaacaagtcaag

SEQ ID NO: 98 - DNA, 102 bp, Artificial Sequence
  Synthetic
agtggaaccaacaactcctcttctgggatgttaacatcagctttgatgttctgatagtaaccgaaagaaatttgtgtaatcgactttgtcaacagccatatt

SEQ ID NO: 99 - DNA, 102 bp, Artificial Sequence
  Synthetic
aagggcacactaggataacccaacaagaaaatttcaagatacaaacaagtagaaatggcgaaaaagtcagctaacttatgtttcaacttttgtcgtaatact

SEQ ID NO: 100 - DNA, 102 bp, Artificial Sequence
  Synthetic
ccattgtatattagtttgacatgaatggttttgttagcatagatctgttgacctttaatagttctgttcatgccacttgttttcagagtttagaatgttagt

SEQ ID NO: 101 - DNA, 102 bp, Artificial Sequence
  Synthetic
taatagttctgttcatgccacttgttttcagagtttagaatgttagtataaggttttaagttcacagtttagtgtggagatattagaaggtattcaagttct

SEQ ID NO: 102 - DNA, 102 bp, Artificial Sequence
  Synthetic
aagctcaatcagatgccatcacttgggtagttgtttgacttttcattttctctatttatgatgttttcttgacaataggtatgaatgtttagtactttgtct

SEQ ID NO: 103 - DNA, 102 bp, Artificial Sequence
  Synthetic
tttttcttttcttttcttctttttgctctggaaacagatgagagagatcatccatttttctcaaggacaaacaatggatcacgaacggggatggaggatggg

SEQ ID NO: 104 - DNA, 102 bp, Artificial Sequence
  Synthetic
ttttctcaaggacaaacaatggatcacgaacggggatggaggatgggtgactgggcgagcaatggaaggatccaacggcgataaaatggcgagggaaggaag

SEQ ID NO: 105 - DNA, 102 bp, Artificial Sequence
  Synthetic
atagaaaaggggtgaacaagaacaacaacaaggctatcattatcttggtggatctttgttgatctcttctttggggttaaaaacctttgcttggagttttac

SEQ ID NO: 106 - DNA, 102 bp, Artificial Sequence
  Synthetic
ctttcaacactaagctccttagctcctgagccgcggcatattccggcgaaacttttccggtagaagacgacggcgttggtaaatataagaagaaggtctcta

SEQ ID NO: 107 - DNA, 102 bp, Artificial Sequence
  Synthetic
aacacgacttagatcttaaccaaaaaaccaaaactcccgactcagatctcgaaacaagaacaaagagatttacacgatcgaaacgtcgcgtcgccgaacctc

SEQ ID NO: 108 - DNA, 102 bp, Artificial Sequence
  Synthetic
tttaagcctcccaccaaccccaacatccccctcactattatctctccctctgtttattggtagttacctccgtcttggccgcggccaccaccgtcgccttct

SEQ ID NO: 109 - DNA, 102 bp, Artificial Sequence
  Synthetic
cgaagccgtcgatggaaaacagaaccgaaccggtgagatcgaacggtccccgagaaatttaaccaacggctcagatttaagacccgagactcgatttttggt

SEQ ID NO: 110 - DNA, 102 bp, Artificial Sequence
  Synthetic
tttgtttcagtttttgttcaaagtttgaatctttattatctgtttggttgcatgagaaaataaggggtttttattttcttggtgggttttcagttcaaaagc

SEQ ID NO: 111 - DNA, 102 bp, Artificial Sequence
  Synthetic
cacactgcaaaagattttaaatcacattttttgtacgatatcgtgcgatggcagataatttatataaacaatttaatcaacaatcctagaagaaagagccta

SEQ ID NO: 112 - DNA, 102 bp, Artificial Sequence
  Synthetic
gtttccaacctcatctgtttcgaaaaagcattttgtgtaattcagtaagtatattcgctaaaggaagagaaaaaagtttccgagttaaagtgtttggaaggt

SEQ ID NO: 113 - DNA, 102 bp, Artificial Sequence
  Synthetic
tttgtatagttcttcaaacacaacaatatcaggttcatttgttatagcatatatattagacaaattataaagaattaagtcactaataagcgatgttaaata

SEQ ID NO: 114 - DNA, 102 bp, Artificial Sequence
  Synthetic
aatttaagagaagaagaagtaaaggaacgagaagaaaacgatgaaatgtgtagaagaacttgaaagcttagaccatgcagctcctttgaatgtggcttttcg

SEQ ID NO: 115 - DNA, 102 bp, Artificial Sequence
  Synthetic
gagataaagtttgaatttttacttacaaattgaggagaggaagtagtaatagaggagatgaagaagaaaaatgttaaattgatttgggtttcttctctttct

SEQ ID NO: 116 - DNA, 102 bp, Artificial Sequence
  Synthetic
atcaaatgtgtcagttcaaaagttgcaaataaataacagtgagaaatggacacgatttggaagaaaaaaaaaataagaaaatctatgtaatttgtatcatct

SEQ ID NO: 117 - DNA, 102 bp, Artificial Sequence
  Synthetic
atctgccaaaaacaagaaaattgaaactatgaatctgttagatatcatgaaggtccttttatttgaagagtttataatcttaatccatataaataaataggg

SEQ ID NO: 118 - DNA, 102 bp, Artificial Sequence
  Synthetic
ggtcgggatgaaaagcaggattctataataggccactacgctttttactaagaaaataagtttctgcatttcattcaactgcattaaaatttagatatattc

SEQ ID NO: 119 - DNA, 102 bp, Artificial Sequence
  Synthetic
ggtttttgagatttaatcttcctctctcattcactccatttcatcagtatgtttataccaattcaaagttctatcttcactctaaaatcaagccaattttca

SEQ ID NO: 120 - DNA, 102 bp, Artificial Sequence
  Synthetic
ctaaaataatccaaagttcatacatggttattctcacacactcacataacgtaagatagcatttcaaatataatagatttgtattctctgtagatagatgtg

SEQ ID NO: 121 - DNA, 102 bp, Artificial Sequence
  Synthetic
cgtaataatcccttcaacatcttaaaaatatctgatttatcgtaatagtaacatactttaaactctttcgaaaactcgattttgatcttctgattcttcaac

SEQ ID NO: 122 - DNA, 102 bp, Artificial Sequence
  Synthetic
aaactactcacataattcccaagagagatagaggccatacaaagtgagcttaccgaagctcttaatcccatctggggattgttcattaaaaaattcaagttg

SEQ ID NO: 123 - DNA, 102 bp, Artificial Sequence
  Synthetic
ttgaggaccttgaacctcatacaactcctcttcacgaactttctcgagattcccacttcttagcagctgcaacaaatacttgcgcgacacgtggaatagggt

SEQ ID NO: 124 - DNA, 102 bp, Artificial Sequence
  Synthetic
catttaccaatatcttcaaaatagaccaaaatagtgtttgagaaaagtgagaccaagatttaatgagaagtaaaaacaacaaaagtatgaagctttgttgtt

SEQ ID NO: 125 - DNA, 102 bp, Artificial Sequence
  Synthetic
ttgtggtctaaattcaaattaataacaaaataaaatagatgaaattattgatatatatatatatatatatgctaaataatgttgacagctaatttctaccct

SEQ ID NO: 126 - DNA, 102 bp, Artificial Sequence
  Synthetic
caaaatcttaaaataagttgtttgaagcatgctatatatacacgtgaaattaagttattttaagtttaattaaagtaccttaaattattaaaaaaattaaaa

SEQ ID NO: 127 - DNA, 102 bp, Artificial Sequence
  Synthetic
aatatcttcaatcccgctttcggtgatagtttccacaatattgttattattgtgtggtattatggcaactattggtgtcattgttggtcatattcaaattag

SEQ ID NO: 128 - DNA, 102 bp, Artificial Sequence
  Synthetic
ccggctagccaaggtggcaatcggattcctgtaaaagggtacaagattccctgccgcaattggcatcccgaggatgttgtagccgagggcccaaacataatt

SEQ ID NO: 129 - DNA, 102 bp, Artificial Sequence
  Synthetic
tgttcactgttcgcctgcatagggaaaaagacatcttacaaaagaaactcacaaaaattcgggctgcagagattttcttgtagtaattcaaatctctaacca

SEQ ID NO: 130 - DNA, 102 bp, Artificial Sequence
  Synthetic
gttggtgttgctagaccgagagcacacgggcaagcaaccaccaaaactgataataccgaattgcagcgcaagctcaaattcatccatgtcttttggtatcca

SEQ ID NO: 131 - DNA, 102 bp, Artificial Sequence
  Synthetic
taattcacagaacaaaaaagaaagtgagaaaaatacaatgaaagttaagttctttgaaacaaacccatgtcaaattcagcagagaaagaacaaacccagttg

SEQ ID NO: 132 - DNA, 102 bp, Artificial Sequence
  Synthetic
cttcttccattaccaaccaatgatgtgcacatctttcttaaccattcttcatttcttttcttcctcatcttcctatatatattatgataatacaatatatgt

SEQ ID NO: 133 - DNA, 102 bp, Artificial Sequence
  Synthetic
atgcattgtatcttctaatcctccattttcattctccaatccacttaaaatattctataacatcctttttcctgcaataacaaagaaattcacttaaaaaaa

SEQ ID NO: 134 - DNA, 102 bp, Artificial Sequence
  Synthetic
taagaaactgcaaaccgccaattgagataaggcgtttagcattgttggttcagctcctcttcgccaaaattagtaaaaagttgctctaagatcaaaagggtt

SEQ ID NO: 135 - DNA, 102 bp, Artificial Sequence
  Synthetic
ttttccagctttctattactcgctttagtattaagtttgttgaagggactatcattgcattccaatacttttcctgttgttgggcatgttttgtttccttga

SEQ ID NO: 136 - DNA, 102 bp, Artificial Sequence
  Synthetic
tcgagaaaatcaagaatagacaaacaactcccgcgaatcattactcgaaatctccttttctttaagaaaccttttgatgtatccacttaagattgacaccat

SEQ ID NO: 137 - DNA, 102 bp, Artificial Sequence
  Synthetic
aacttcatcaagagcaaccttctcataactcaacttcttactttgaactttcttcaaacactttttttcccttaacttcatccgaaaatatgtcattttctt

SEQ ID NO: 138 - DNA, 102 bp, Artificial Sequence
  Synthetic
tttcccttaacttcatccgaaaatatgtcattttcttcatcttcatcttcagtcttgaacctcaacaatctcattatttgaatgtgaagtgattgattctct

SEQ ID NO: 139 - DNA, 102 bp, Artificial Sequence
  Synthetic
cgagaggccaaagagtgtctttacgttgaaccagatgatgaatcttcgaaagaaaggtgttctttggtacaagaacaaactttgatcaaatcggcagtggaa

SEQ ID NO: 140 - DNA, 102 bp, Artificial Sequence
  Synthetic
ataactaaaggtggcaccgcctatagaaggtgttggacaccactaataccctaataacaatgctcttattattacttcctaacgtaccataatgtttgcatt

SEQ ID NO: 141 - DNA, 102 bp, Artificial Sequence
  Synthetic
tattacttcctaacgtaccataatgtttgcattaccgaaagaaaaagaactagggacattatcgtccaaagaaagtttttattgattttgaactgtctcctc

SEQ ID NO: 142 - DNA, 102 bp, Artificial Sequence
  Synthetic
aaaatagcacatttaatatactagacaagtatggtgcttgtttgttgattgaaatttaaaggttttgaaaaggtagaagatgccattaaaatctagattgag

SEQ ID NO: 143 - DNA, 102 bp, Artificial Sequence
  Synthetic
cctattcggattttcagctacggtagttaagcggccagtttagtgtgatgcttttagccgaagggtaaaacagtattttcataaaccgaaaaacggacctct

SEQ ID NO: 144 - DNA, 102 bp, Artificial Sequence
  Synthetic
ttgtcttttttcagcttctgaactttggttttttgttgttgaaatgcttactagggaagattttgattggagtttgagcaataatgacttctagagggaggt

SEQ ID NO: 145 - DNA, 102 bp, Artificial Sequence
  Synthetic
tttatattttttggtcatgtacacaacaaatcatcttggcaaatggtttagatatggaaatgccagttaatgtatcatgagtttaggcttagtttgtaaatg

SEQ ID NO: 146 - DNA, 102 bp, Artificial Sequence
  Synthetic
ttaggcttagtttgtaaatgcatcttctttcgctctgtgcctgcttaaggctctttaagtatctcctttcagcgtacttctatattgtcttccttgtttcta

SEQ ID NO: 147 - DNA, 102 bp, Artificial Sequence
  Synthetic
gctttgcaaaatgctgcagataaagctgaccggtgggttatattttatgtcttgggacgatcactttggtttttgcattagttgacaatcaatcttttgctg

SEQ ID NO: 148 - DNA, 102 bp, Artificial Sequence
  Synthetic
aagactcagattctagttccatataatattcttccactagatcctgatagtcgcaaatcaggcaattatgagatatcccgaggtatgtttgtaacattcagg

SEQ ID NO: 149 - DNA, 102 bp, Artificial Sequence
  Synthetic
taatattttctgttgttaggagtcttttaccagccatattcaatctattgtcttcctaattatgagtatgtagaacttttagactactgacattgctgacaa

SEQ ID NO: 150 - DNA, 102 bp, Artificial Sequence
  Synthetic
ctcttcaagaactacaaaaaatggtgcaagtacttgggtcgtaaaagtagctctttggtgagtgctttttgtgctgagctatttgtcattagattttgaatg

SEQ ID NO: 151 - DNA, 102 bp, Artificial Sequence
  Synthetic
caagaagttcagcagcgcaaacttctatacatgggactgtatcttttaatattggggggaggctgcaaatttaagatttatgcccgaatgcctttgttatat

SEQ ID NO: 152 - DNA, 102 bp, Artificial Sequence
  Synthetic
tctttttttgatgcatatgggaattgaatgaacaccttcccctaactcgcgacatacccctcctctcataccatttgagtttacctttgttagttccaattt

SEQ ID NO: 153 - DNA, 102 bp, Artificial Sequence
  Synthetic
agagtgtgcgagggccatcctatattgagatcattcgcattatttactaatcctccttgattaactttccagtgttgttgagcatatattcaatgtggttga

SEQ ID NO: 154 - DNA, 102 bp, Artificial Sequence
  Synthetic
gcagatggtgatctacttactgaattcaagatgagttccctccctacacttctatgggcactttgttaagcttattgaatttatggtaagttttgaatgtcc

SEQ ID NO: 155 - DNA, 102 bp, Artificial Sequence
  Synthetic
ttgtggcggaagaatatcgtgattacaagattttagcattttaaaacactagaaacatacatcaaccaaaaataactcataggaagcaattttttagtctct

SEQ ID NO: 156 - DNA, 102 bp, Artificial Sequence
  Synthetic
ttggtttgttttcttatgcagctgagacgtgtgatggaaactgatgcagcctatgacggacaacttaattgcttataatattattccacttgatgctcctag

SEQ ID NO: 157 - DNA, 102 bp, Artificial Sequence
  Synthetic
ttgtacaacatttacatgggcactgaaagttcattttaatttcttggtgatagtgatgatggaaaaatttcttatctgttgctgagttccacaaaacagatg

SEQ ID NO: 158 - DNA, 102 bp, Artificial Sequence
  Synthetic
tttgtttttttatatgtttgaccataaccttacaccttcaaaatttctgtctgtcatctccagattagatccttggaattagttcacaaactttttgaagag

SEQ ID NO: 159 - DNA, 102 bp, Artificial Sequence
  Synthetic
attatacctcaactgttgtggatttttaattcgagggtttcaatatattttatcttttgtaacaccaatctctcccttaatgatgtttcttgtttcctctga

SEQ ID NO: 160 - DNA, 102 bp, Artificial Sequence
  Synthetic
gttagaaatgaagcccttcgtgttgcttttattgatgaggttgaaacacttaaaggatgatggtaaagtacagagggaatatttctcaaagctcgttaaggg

SEQ ID NO: 161 - DNA, 102 bp, Artificial Sequence
  Synthetic
tgcttttctttccgttcccaagttctccctttctggtagattacttagtgcgtatatggtatataacatatagggatttataggctgaaaaagtctaaatta

SEQ ID NO: 162 - DNA, 102 bp, Artificial Sequence
  Synthetic
actattcttagtctaaggttctttattttccagtttggtgttgtctacaagactagatttgcaaggaaacaatacatcgctttcggtattacctctattcat

SEQ ID NO: 163 - DNA, 102 bp, Artificial Sequence
  Synthetic
atcccgattgcattcttttcttggttcccattcgtgtcaacattccaaacacagactcatgttcaaccaggctttcagccgaggtttggagatctctctcat

SEQ ID NO: 164 - DNA, 102 bp, Artificial Sequence
  Synthetic
caaaaataaataagattttaatttaatttaaaaccaaaaaagaaaaaaaaattaaatcctctaaaatcttcttttgaaatccataactattcggcttccacg

SEQ ID NO: 165 - DNA, 102 bp, Artificial Sequence
  Synthetic
tcctcttcgccatggctataataaggtttggtcttctcaacaacaacaaccaacaaaaccagcaaaatcaacagaagcaagacaagtttcagaattcatcga

SEQ ID NO: 166 - DNA, 102 bp, Artificial Sequence
  Synthetic
ggagaatggtacttgttcggagaagaagagcattggtgaagaggattaattgtaagtctgcatcttctgaacagataacccaattggatttcggaatatttt

SEQ ID NO: 167 - DNA, 102 bp, Artificial Sequence
  Synthetic
tggacataaaatgatgatgatgatgccttttgttatgtaaatgaggatattaacattttcgtaagaactggctttgttctccaaattctaccttagcaataa

SEQ ID NO: 168 - DNA, 102 bp, Artificial Sequence
  Synthetic
accaccaagagtttggtggagagctacaacaataatcaaactcttttcttaggttgattttgtcaaagctatgattaagatgggtaatattagtcctcttac

SEQ ID NO: 169 - DNA, 102 bp, Artificial Sequence
  Synthetic
cttttgtttttgtttttaagagaaacgacacaaacattagaaaacgacactgtaaaaacagaggtgggttacaaaatgattattgagaaactgaactgtgct

SEQ ID NO: 170 - DNA, 102 bp, Artificial Sequence
  Synthetic
tgtcttccttcttctgggttctttttcttgtttgagttcacacccttgtttaatattatgcataattttactctgtaaggctaagaaaccagagtagaaaag

SEQ ID NO: 171 - DNA, 102 bp, Artificial Sequence
  Synthetic
gacgatcatcgcttgatcacatacccattgcttactattatccatggtgactgatgatgatgatagtaagaaggatcatttaagaactgccacagcggatct

SEQ ID NO: 172 - DNA, 102 bp, Artificial Sequence
  Synthetic
atgttcaccctagccggtggtggagtcgagcccttcctctgcgcaattttctgcaggaggtaacttcacactaggcttgggcctgctttgtttctgggattt

SEQ ID NO: 173 - DNA, 102 bp, Artificial Sequence
  Synthetic
caagctaatttccaaaaagataacgaaatggctttgattaagaacttttcatgcacaatttaatacctttccacatccctcgtaatgacagacatattgtct

SEQ ID NO: 174 - DNA, 102 bp, Artificial Sequence
  Synthetic
cccattctcttatatcagtcgacgtcgttttattactaatctccatagctctttctcccacttaaaatctccaaaaccaaaacgccaaaactaaacacgtcg

SEQ ID NO: 175 - DNA, 102 bp, Artificial Sequence
  Synthetic
gaaaatgtctctcaaaaaatatatataaataagagaaaatgggtggtgtgtatttttggtcaaatctatgtttaattattaatttaattcatgcacagcatc

SEQ ID NO: 176 - DNA, 102 bp, Artificial Sequence
  Synthetic
ctgagttgctcgctgtcaaaacaaactgaaacacgcattttgttttaccaattgcaatgagaatacatccacttcaatttaaacaactgggaacagaaaggc

SEQ ID NO: 177 - DNA, 102 bp, Artificial Sequence
  Synthetic
tctaattttctcgtcaaccaaacagaaaaagtaataagaaaagacaataacttgagagacctgaagtagaaaacaataacccgatgaagacaacaagcttct

SEQ ID NO: 178 - DNA, 102 bp, Artificial Sequence
  Synthetic
ccgaagctaatccaaacactttaattgcttccatttttgatcaaaactgagaagacttcgaaagcaacagaaaaaagatagagaagaaacaaaagcagtgaa

SEQ ID NO: 179 - DNA, 102 bp, Artificial Sequence
  Synthetic
tccacatcaagtcttacttgattaagctgttttctagcttcctcgcattttcagttcattttctttttcaagttgctttttgtattcctccaactgctccac

SEQ ID NO: 180 - DNA, 102 bp, Artificial Sequence
  Synthetic
tcaatctatgtacatgaaaagactaagtatttcatttattccttcagtgagtacatttgataagtcttaggcttatcaatacttaccctgcattttacattt

SEQ ID NO: 181 - DNA, 102 bp, Artificial Sequence
  Synthetic
aatccaagatgacccaacccagatcatcatatactactaattgttcaaaaagtattctcccttcaacaaaatattagttcacaaagagagtcaagtgaagaa

SEQ ID NO: 182 - DNA, 102 bp, Artificial Sequence
  Synthetic
tgaaagcccattacatctgttggaaatcaatccaaccccaatctcaactctcctgtacacataaacacagcaaagcacaaaacagcgcagcaaatccaagaa

SEQ ID NO: 183 - DNA, 102 bp, Artificial Sequence
  Synthetic
taagaatgaaaataggaatataaataaaaatggaattaaatttaaaatgctatatattaatttgtttatcatcttgcattagaatggtcttttcttccattt

SEQ ID NO: 184 - DNA, 102 bp, Artificial Sequence
  Synthetic
cccgagtttgttgtgactcggccgagttcgaactcagttcagcattgatttctatatcttatcattgaagtttcagtcccaaacaataatggttgttgtggc

SEQ ID NO: 185 - DNA, 102 bp, Artificial Sequence
  Synthetic
tccgttggattttactataattttgtagtttttgtatagatggtgatagttgagaaatgagatttgggaattttgtgatagtgaagaaggggatagttgaaa

SEQ ID NO: 186 - DNA, 102 bp, Artificial Sequence
  Synthetic
aatttaatactttcattattggttacactgattagatagagtggtaggctgatattggatctgttctactagctctgcggttgttggtgcttaagtttgatg

SEQ ID NO: 187 - DNA, 102 bp, Artificial Sequence
  Synthetic
aggctacatacaaattatacctaccatgacatgttgaatttacagtatttctagtgctgatgatcattttatttgcagtgctgtacttgcacatattattct

SEQ ID NO: 188 - DNA, 102 bp, Artificial Sequence
  Synthetic
tttgttagttttgcatcaatgagaaacatccctgcatatgaaaatgctgttcaaggagtccttcgaacgatgcttggatctttacttgtgtcctagagtacg

SEQ ID NO: 189 - DNA, 102 bp, Artificial Sequence
  Synthetic
aatcctgtgcgggatctttctatactggctgcttctacgtaaggccttattgctactgttaggttttgcatctttaagtaaattaacatttgagtatgatta

SEQ ID NO: 190 - DNA, 102 bp, Artificial Sequence
  Synthetic
gacactgtgtcaattgcaagttggcatcaagatgacaaacacggtggaatctagattgaagcattttaaggtgtgttaataattgcccttagttttttcttt

SEQ ID NO: 191 - DNA, 102 bp, Artificial Sequence
  Synthetic
aggtgaaacttcttttctttaatataatttattcatcaaacaaaaaaacatctatggatcttgcttaaatgtgtatcgaattccacatataatgttatttac

SEQ ID NO: 192 - DNA, 102 bp, Artificial Sequence
  Synthetic
tgtcacatatttagattattacaaaaatgtgattcccacacgtataaaatcttgtatggaatttaagtgcatctctagatgtattaggataccaaattgaag

SEQ ID NO: 193 - DNA, 102 bp, Artificial Sequence
  Synthetic
acagcctttacaactgtacatttatgaaaaaacattagagatacaacaaaagagataaactagttggaaggaataaaaacacaattaggaataaggatggaa

SEQ ID NO: 194 - DNA, 102 bp, Artificial Sequence
  Synthetic
agataacaaagaatcaaatattcctaattacccaagaaagaaaactataatatatctctagctaaaaaaaataacaaaattcttaccctaagaaatatggtc

SEQ ID NO: 195 - DNA, 102 bp, Artificial Sequence
  Synthetic
ggatgctttagcatagcatacagaaacacatacgatgccatttattatttcactctcttaaaacaaacttatattccatttacatcatgtcacttaagtgcg

SEQ ID NO: 196 - DNA, 102 bp, Artificial Sequence
  Synthetic
tattactatatatctacccaatgtcgtatttacagggaaaaaagcttgactggtgggctgatgaataattttacatggaaactttagatttgtatttgaagt

SEQ ID NO: 197 - DNA, 102 bp, Artificial Sequence
  Synthetic
taacttacaaaaaggaaaacctttgaaaagacatgctttcccaagattcatcgggttagttcatcttggagggttgattcactaaacgcgtttgtgtttcta

SEQ ID NO: 198 - DNA, 102 bp, Artificial Sequence
  Synthetic
gcagatattgggctcatcagttttccaaaaaatttccaggtatgtggtttctagtatcattcaagcaacatagcctaactggttttaatgccatggtaaagt

SEQ ID NO: 199 - DNA, 102 bp, Artificial Sequence
  Synthetic
taaaggtaatacatgtgatttggttgtactgcttatggtaattccaaattctctttcccctttccaaaaatcccacattgcttctggtgagatttgatcata

SEQ ID NO: 200 - DNA, 102 bp, Artificial Sequence
  Synthetic
tttagtactagggtacatgatattgtgcttctattgcttgcacactcaacctgcaaatgctctttttcttttccttcttagaggagtgtaccactgtaaact

SEQ ID NO: 201 - DNA, 102 bp, Artificial Sequence
  Synthetic
accgatgcaataagagcaaagatagctcaagaggagtacaaagaacatcaagaatgctaggcaagaagcattgcagagaaagaaagccgagaggagaaaact

SEQ ID NO: 202 - DNA, 102 bp, Artificial Sequence
  Synthetic
taccggctgagctaagacggcaatgaatttttcaattaaaattttactatgctaacacagtgcctcatcgagccattgggcctagttttaatcggcccattt

SEQ ID NO: 203 - DNA, 102 bp, Artificial Sequence
  Synthetic
tggctgtccagacaaagcttccacacagcttcattcccactcgaaccttcagtcaacgccagagtcaaatgggtacgagacccatacctagatttcgcagta

SEQ ID NO: 204 - DNA, 102 bp, Artificial Sequence
  Synthetic
gagaagctaaaatgggatatgggtcttcctcacaatttcatatcttcgctctgtaatcgattttcccgattactttcaggtttgcgaaattgaaaaccctaa

SEQ ID NO: 205 - DNA, 102 bp, Artificial Sequence
  Synthetic
gactcaacaagacactgaatttgaagtgagtttaatcatggtggttgtgctctgaataatgtgcatgcatatttgctttaatgctcatatttggtctttgca

SEQ ID NO: 206 - DNA, 102 bp, Artificial Sequence
  Synthetic
tttaaaattggattatacagaacttccctttattgaaatatagcttggccagcatttactatcaacatttttccactttagttttttgtcattttctgtatt

SEQ ID NO: 207 - DNA, 102 bp, Artificial Sequence
  Synthetic
tgactttgggagctgtttcattgcagcatcatttagataatcaatccttaatggttatagcacagagaagatgttttctttgaaacgggtgcaatcatacca

SEQ ID NO: 208 - DNA, 102 bp, Artificial Sequence
  Synthetic
aatttttttgacaataattgttggtctataggggagagaggactctgttaactgactctcccaataaaggactcatattataaatagaagcattatagtccc

SEQ ID NO: 209 - DNA, 102 bp, Artificial Sequence
  Synthetic
tgctgttgatgctgaagttgctgttgcctgttaaatccatgctgcatagagatctctctcttgattgggggttaccacagttgacaaaggcatttgattaac

SEQ ID NO: 210 - DNA, 102 bp, Artificial Sequence
  Synthetic
gcttcagcaaagctattggtattaacagcatacaatgccaatccaccaaaacccaataaggaacatagttgcaaatagaagagcaagcagcttggcataagg

SEQ ID NO: 211 - DNA, 102 bp, Artificial Sequence
  Synthetic
gttcatatctatacaattacaaatctaaacgaaattaaacaacagatccagaatgaaattgatatatgattaccttaagttgctcaactcttccatggagat

SEQ ID NO: 212 - DNA, 102 bp, Artificial Sequence
  Synthetic
agtaaactagcatcggatgtattggctatatatcagattggactgatatttcacagcagaaaagatatcgtaaacttaccgtaatatattttctaagtaaat

SEQ ID NO: 213 - DNA, 102 bp, Artificial Sequence
  Synthetic
tccgaatccaggtgatcctactacaccagggcttagactgagtcgactcacgtagaagaagaacgattaggaacgagtttcaccttctcactcttcttacac

SEQ ID NO: 214 - DNA, 102 bp, Artificial Sequence
  Synthetic
tttcaccttctcactcttcttacacgacggagcagagatccgatgaggaggttcgcgaatcggaggaggaagggagtttgtgagccacgcgcttcgttgttt

SEQ ID NO: 215 - DNA, 102 bp, Artificial Sequence
  Synthetic
taacatataatcatactactaattactatattaattaccttaatttcggggtttaatcggagttctagaagagacggcggccggagcaaagatgcaggagtg

SEQ ID NO: 216 - DNA, 102 bp, Artificial Sequence
  Synthetic
cagtgatacattacattgttgaacccttccttaagaaaaactggtaaagtagacagtatcaaccacagttcccaaaacattggtctcaacatcattgtagat

SEQ ID NO: 217 - DNA, 102 bp, Artificial Sequence
  Synthetic
gtagagaaacttacaaaaatattagattttgggcaaatatttacaattttcactaccatatggcaaaaagagaaatttgctaaactaattatttgaattact

SEQ ID NO: 218 - DNA, 102 bp, Artificial Sequence
  Synthetic
tatattattagaagtttgacccaaataatgtgatcattttagtaattgtagagtcattaaatattaaagtgtcataaactaaatatgtaaatactttaaaag

SEQ ID NO: 219 - DNA, 102 bp, Artificial Sequence
  Synthetic
cagaaaccgataaacgaattaaaagagcaagaagaagaagaaaccctaaatctgagttgaagagagtttttgtagtgattaaaatatgccattgtttgatat

SEQ ID NO: 220 - DNA, 102 bp, Artificial Sequence
  Synthetic
tcttgaatacaccacaatgcaaccattacaaacctctctactctgttcatgctcacccaaggcttcttcatcattcaccaaaaccatatccattttcccatc

SEQ ID NO: 221 - DNA, 102 bp, Artificial Sequence
  Synthetic
actgcaaagaaactggtattggaaggcacaaatctgcaccaaggacaccactagcagaatggcttggctgagaggatgaatagaactcttattgaaagagtt

SEQ ID NO: 222 - DNA, 102 bp, Artificial Sequence
  Synthetic
tacatgtccacaatggcattacccacatagacatcttcattaaaaccatttacggacagcaaaaccatgaacttccttaccccacattgataaacccataga

SEQ ID NO: 223 - DNA, 102 bp, Artificial Sequence
  Synthetic
cctaaggtttcgggaaaggttgggatgtatgtctgtggagttactgcctatcgatcttagccatctcggccatgcccgagctgccattaattttgatgtcct

SEQ ID NO: 224 - DNA, 102 bp, Artificial Sequence
  Synthetic
ttcttcctaagatcttatttatgggattgtattttaatataatattttttctcatcgtactttgcagatcatcaagaatgactatgcatatgcagttgatgg

SEQ ID NO: 225 - DNA, 102 bp, Artificial Sequence
  Synthetic
ggtggaattgacttgatctttccacatcatgaaaatgaggttgcccagagtctgtgctgcatgccaagagagcaatgtaagttactggatgcataatggaca

SEQ ID NO: 226 - DNA, 102 bp, Artificial Sequence
  Synthetic
tggtaataagcaccaagccagtgtggatattgagctcaagggtggatcctctttggcacagtggttgcatttgcatgccaaactatggttacatcaggaccg

SEQ ID NO: 227 - DNA, 102 bp, Artificial Sequence
  Synthetic
gtccaacacagcagcatcaaaagaaatagtaagcttagaaatgtttggttgaagatgtgtactactcatcatcattgtgtcttaataatataactaataata

SEQ ID NO: 228 - DNA, 102 bp, Artificial Sequence
  Synthetic
agagcgaggccacgaattggtggactatatcccaggaatccgttcaactcgagctatcagatatctcagagaatttttttgagaaagaagatagtaaaacaa

SEQ ID NO: 229 - DNA, 102 bp, Artificial Sequence
  Synthetic
tggttcattagtcttgttaatctcagggagagttaatttaatgaaccaatacaataatgattcaagtggattaaggccttatttgctagggaagttaaggtg

SEQ ID NO: 230 - DNA, 102 bp, Artificial Sequence
  Synthetic
aaaaccatttgaatgattagaattagaagatgacattggtatcaactcatctaaagatggttgttccattctgttcttgcaagtaaatagagccagattggt

SEQ ID NO: 231 - DNA, 102 bp, Artificial Sequence
  Synthetic
ttcggttatcactaggttggagattagagagttttgttatgattatgaaagcttctttgtgtggccccttggataaattgttagcttttgcagttgagaata

SEQ ID NO: 232 - DNA, 102 bp, Artificial Sequence
  Synthetic
atcaaaatcaagagttgctctataatagtaatttgcaatagaagcactctcttgtactaaacatatccatttcattattctctctcagtatatacaaactac

SEQ ID NO: 233 - DNA, 102 bp, Artificial Sequence
  Synthetic
ttcagtgatgaaatctttttacttcttctactaccattcttttggcgtaagaaagaagaatcctacacaaactgtggctgccaataaaatattaacaaaaac

SEQ ID NO: 234 - DNA, 102 bp, Artificial Sequence
  Synthetic
tgatcaactcatcttggtttttcctcctctcttctacataaggaatcccatactaaggtgatattgtctttacgaactttgataaacgccctcgagttaaga

SEQ ID NO: 235 - DNA, 102 bp, Artificial Sequence
  Synthetic
actgatataagttgttaacttcccctctcaatttagctgcttttgttggagagaaaaatttagagaggaatttcatcggcaagtcaccacatgtgttgatag

SEQ ID NO: 236 - DNA, 102 bp, Artificial Sequence
  Synthetic
tttgtcccaccaacaatgtgtctgttggttcatcgaaactctcccacaaatctggttttgttgttgtttgctaggacaagattacctgtgtctaacacagcc

SEQ ID NO: 237 - DNA, 102 bp, Artificial Sequence
  Synthetic
cagataaagaattccaagttgaagtgaatgttataggccaaactcaccacagagaatcttgttgggttgaatggatattgtgatgagaataatcataggttg

SEQ ID NO: 238 - DNA, 102 bp, Artificial Sequence
  Synthetic
gtcttaaaccaagttgaaatcaaaggactctaattgctgttggcattgcatgacaacactcagatcaaagccaatttaatactaagactatgattagaggaa

SEQ ID NO: 239 - DNA, 102 bp, Artificial Sequence
  Synthetic
tttttgtgaatgttcaatggataaattcagactcaaatgttgatgaaaaaaggcaaggaaaaactagcactagagatgcttagaaagtgctctggtctgcca

SEQ ID NO: 240 - DNA, 102 bp, Artificial Sequence
  Synthetic
tctggtctgccattagctatcattgtgctcgctgggcttctttctaagaaatcacaccttatatgattgggagctgatgaaagaaaatgtaattcgctgcat

SEQ ID NO: 241 - DNA, 102 bp, Artificial Sequence
  Synthetic
caccacatggaaatcatcacatatatctctaacctcttcttatcacccaaagacatcatcttcataatccacaagaacatccaatgacccttctctgaagca

SEQ ID NO: 242 - DNA, 102 bp, Artificial Sequence
  Synthetic
agtaatcccttgttgacactactcttccttgtgttaaattgaacctttcgctcatttcctctcgaaatatacaagtagcccgtttcgttgaagattatctga

SEQ ID NO: 243 - DNA, 102 bp, Artificial Sequence
  Synthetic
ctttacattcaactcagtaactacttatgattttggtctctaaatttgtgtctcaactaatcaaagaccaaactcaagtacagtggagtaacctggtgataa

SEQ ID NO: 244 - DNA, 102 bp, Artificial Sequence
  Synthetic
ccgttacctagtcctactgtctcagcatctcgtaaaaaacagaaaccatcagcaatctgtagcttcgatgtctattggtgctccctcgccagcgctgcctcc

SEQ ID NO: 245 - DNA, 102 bp, Artificial Sequence
  Synthetic
aaaacccatcaacccaccaaattaaattcaaataaaaatccaaactttaagaataaacaagatcactcactctatcaaatcacagatccccaaattgaaaga

SEQ ID NO: 246 - DNA, 102 bp, Artificial Sequence
  Synthetic
cgaacccagttacaaacagcaacaacatctttgatttcagagaatccggtagagagaagctcttccggtggattttccggcacctctagaatcaaaggtgac

SEQ ID NO: 247 - DNA, 102 bp, Artificial Sequence
  Synthetic
agctcattaactccaaactcatgctgatcatttgtacttttcctcccaatcaactctttcttcactcctctttttcttaatacccttttcatcaatcgaaac

SEQ ID NO: 248 - DNA, 102 bp, Artificial Sequence
  Synthetic
tttcttcagaagacttattactccttttaccacaagttactataatagatctgagaagacttgtttgatttgggagctgatgaatctgttttctctacaact

SEQ ID NO: 249 - DNA, 102 bp, Artificial Sequence
  Synthetic
aacctgtctttacagttgtctggactccgatacacagaggcggttcggttaggagatttccatggcgatggagtcccagttcttggtgccgtaccggttaac

SEQ ID NO: 250 - DNA, 102 bp, Artificial Sequence
  Synthetic
tcacgtacttttttgggttgttgggccagcccgattatgatttttattttatattttatttatatttattattgggtaaatatatttttctattattgagga

SEQ ID NO: 251 - DNA, 102 bp, Artificial Sequence
  Synthetic
taacgacttttccgggttcggattcgggtcgtggaggatccgaatcatttcatcctctctggttcgggttccgtttcggatttcacggcgccgtttcgtcga

SEQ ID NO: 252 - DNA, 102 bp, Artificial Sequence
  Synthetic
atatatgtttctatgtatatttggttataaagtattgatttttctgattgtctaatgaagaaaaaagagatgagttggcaaaaccacagtcaaagaaattca

SEQ ID NO: 253 - DNA, 102 bp, Artificial Sequence
  Synthetic
gcaacaatgaggaaccctagaacctatacaaaaagatctaggccaacagaatacttctcaaccagagaaggtaaaaagttttcatttttaacttttaatgaa

SEQ ID NO: 254 - DNA, 102 bp, Artificial Sequence
  Synthetic
ataactgttgacaaaaatagtcgccaactcgtttgtaagtctactctacttgtttatcagattttaatagtcattagttgttgtgtttatccaccttaattg

SEQ ID NO: 255 - DNA, 102 bp, Artificial Sequence
  Synthetic
taataaaaattcgaggtgtaaaatatgatagtggtcataattcgagaggtacaaatagctaattatttaacgaaagtccttcgagaaaaatatttaacattt

SEQ ID NO: 256 - DNA, 102 bp, Artificial Sequence
  Synthetic
atcatcagcaacaccagcaataaaagctctcttacctaaacaaaacaaaactccacttatcatattagcaaaaatgtcacattattaaatttcattgagacc

SEQ ID NO: 257 - DNA, 102 bp, Artificial Sequence
  Synthetic
cacaaacgggtcttgacagctggaggagaggcgccatctgggtaagatggtgggaactacagggttcctattgggtggagggagggctgggctgccaccagc

SEQ ID NO: 258 - DNA, 102 bp, Artificial Sequence
  Synthetic
acctagcaaaagatatgcatatttccaccctcctgatgatcttgttgttaaggttcttcttctcataattttcagattcatcatcatctccaaaacttgcat

SEQ ID NO: 259 - DNA, 102 bp, Artificial Sequence
  Synthetic
tcatcataagtcctttcgatttctaaaaacaagaatatttactctcttatgagaaaagaacaatgcacaaacttcataatagcttacattaatcaactctgg

SEQ ID NO: 260 - DNA, 102 bp, Artificial Sequence
  Synthetic
catagcatataaggcaagaagaagtggatatccataactttcaattttatcttcaaaatattatcaaattatctaaatcacttcaaaaatgacactctaatg

SEQ ID NO: 261 - DNA, 102 bp, Artificial Sequence
  Synthetic
atcactgattgtggattactgctttgataattttgtgtgatttgatcaccagcgtcaaaaagaaaaaaagatgaataaacacttgtcttgccagaaaagagc

SEQ ID NO: 262 - DNA, 102 bp, Artificial Sequence
  Synthetic
ttaggttttgcatattctaatgtatagtaaatattatgaaacatcattgtctattaaattttagtggtgtttaattgagatgatgcccatggctaacataaa

SEQ ID NO: 263 - DNA, 102 bp, Artificial Sequence
  Synthetic
aataatatatcacgtgatactggacactattcgtagtctttaacaaatcgtctaaacacttgtaacacgtcacctataatataattagagaccacatgtgca

SEQ ID NO: 264 - DNA, 102 bp, Artificial Sequence
  Synthetic
agttaatttatgtattttggttgtttttgttatttttatgtttaagttggattttttgttatttgatgtttttatgaaattaaatgtcgaattaatgtagta

SEQ ID NO: 265 - DNA, 102 bp, Artificial Sequence
  Synthetic
tggcacatgttagttcagtgacggactcaaaaattttgatttaatagaagctgaaaattgaataaatatgtgtatcaataaggacataatattagattgtcc

SEQ ID NO: 266 - DNA, 102 bp, Artificial Sequence
  Synthetic
tataatatttcttcacatagctttttcactctctcttgtcacaccacaaaagaataatattacatgctaacacttcttacatcttcttaaagttctcttcaa

SEQ ID NO: 267 - DNA, 102 bp, Artificial Sequence
  Synthetic
gagggagctatcgacacttcgagaatggcacgattgatcctttcggtttactatttgtatggaattgtgctcaaagaggaaggcaatgagaatcttgctcgt

SEQ ID NO: 268 - DNA, 102 bp, Artificial Sequence
  Synthetic
ccgaatgattctcggttgtggatcgccatggcccggtgttatgaaaccgactcagcttcgaatgcttgatgaagcaattaagtgttacaaaagggcagttaa

SEQ ID NO: 269 - DNA, 102 bp, Artificial Sequence
  Synthetic
gttacttatgcttatacttgggagaaccctcctggatttgctcaaacaatataaaaattggtttggaaacaattcaagttcgccttccttgtttattttggc

SEQ ID NO: 270 - DNA, 102 bp, Artificial Sequence
  Synthetic
ataattatataccttgcttgttttcatctgattaatgacatgctgatgagctgattatacatgtgtgtcttggatacagataaaacctctagtaggaccgac

SEQ ID NO: 271 - DNA, 102 bp, Artificial Sequence
  Synthetic
gaatctgccatggatgtgccatccaatttggaagcaagaagaagaatgtcgctttttctcaaattcattgtttatggatatgcctgatgcccctaaagttcg

SEQ ID NO: 272 - DNA, 102 bp, Artificial Sequence
  Synthetic
gaattgcagtacagatcagatgtgtatcatgtaattctttgatgttttggctgtgttctgttaagtttgctaaaacagtgtgttatcttattgtatgaaaat

SEQ ID NO: 273 - DNA, 102 bp, Artificial Sequence
  Synthetic
gtcgatgattggactgattggaataagtggataagcaacagaggaggtatctggtgttccgcctgaaaagagttgggaatcatggtgggaggaggagcaaga

SEQ ID NO: 274 - DNA, 102 bp, Artificial Sequence
  Synthetic
cttcagcagttgactgaggaaattcctgatgaggtttgttgtatgtctttgattatcagttgtatttttgaacatccgaaattttgtgaagcatttgtgttg

SEQ ID NO: 275 - DNA, 102 bp, Artificial Sequence
  Synthetic
gccatggggacctttgtctcatggacaggaggggctgttaaatgagtatactgcttaaagagcatagtgaaataaagttgtgatatatcttatgggggtgct

SEQ ID NO: 276 - DNA, 102 bp, Artificial Sequence
  Synthetic
gaggaagctttgaaaatgagaaacctgcttgaagaattccaccgtgatcactggcattcgtcctgccacaattcttggtgttagggagcatgtctttactgg

SEQ ID NO: 277 - DNA, 102 bp, Artificial Sequence
  Synthetic
atatttggcacaagaacttgttaaataatatgaaatgggacgttcattcataatcttcttgattcttattcaaggactagtagagagtctaactagtagcaa

SEQ ID NO: 278 - DNA, 102 bp, Artificial Sequence
  Synthetic
gaaaaaagcttacttagcagcaaaatcacctctaaaactcccttatctgtaggatatagcagagagcaacttttgcctgatttctgtcttcttttgtaacac

SEQ ID NO: 279 - DNA, 102 bp, Artificial Sequence
  Synthetic
tgccaagtgatgttgtggagaaaatcatgttgttggtgcctgcagattctgctagtagagtgtaaatctgtgaacaagttctggtattcttgtatttctact

SEQ ID NO: 280 - DNA, 102 bp, Artificial Sequence
  Synthetic
attttcttatgttgacttggtcaatggagactttattcgagtgttgtctaactatatatatacatacataaatttttttatatatataaataaattgtaaca

SEQ ID NO: 281 - DNA, 102 bp, Artificial Sequence
  Synthetic
ggcgaaaccatcgagtggctgctccagcaagccgaacctgctatcattgcaggccactggtaccggaacgattccggctaatttttcgagtttgaatatttc

SEQ ID NO: 282 - DNA, 102 bp, Artificial Sequence
  Synthetic
agaccttctaatattttaccggctactgccatgtgggcagtagcgccagcctcaagctagtggagctggcagcaccttttggatgctgccagtctcggctgg

SEQ ID NO: 283 - DNA, 102 bp, Artificial Sequence
  Synthetic
ggagctggcagcaccttttggatgctgccagtctcggctggagctggagttgtctccagttccagtagctgctgctgctgctgttacggtcagtcagcagca

SEQ ID NO: 284 - DNA, 102 bp, Artificial Sequence
  Synthetic
agtctcatcatcagtaatgtcagttggaattaaaacatttgaaaatttagtaatgatttataagatgcaaaaaagatgatagctttagagcttacgataatg

SEQ ID NO: 285 - DNA, 102 bp, Artificial Sequence
  Synthetic
aaaaagaatgcccagttagattcaaaaaagcttagccgtttgtccagttctcacattatgaagattccatttgtaccttacaatttcaaaggagaaattagt

SEQ ID NO: 286 - DNA, 102 bp, Artificial Sequence
  Synthetic
ttagatggaaaagaaaagtataatgttatttcgttgaataggactacactagctaggcaaaatattgaatttgaatataaatcttcaactgcatacttacat

SEQ ID NO: 287 - DNA, 102 bp, Artificial Sequence
  Synthetic
agtttgggatcttgccatggagccaggtaactatagattttcatgctgcatgtatgatgcaaattttcagtcatatatattgttgtttcattagatgctcca

SEQ ID NO: 288 - DNA, 102 bp, Artificial Sequence
  Synthetic
agaacaaatataaaatgagcctaattcctttagagaagctgaactgcagcagattgccggaattttttccctcaatttctgtgtaacagcaactcttacagt

SEQ ID NO: 289 - DNA, 102 bp, Artificial Sequence
  Synthetic
ttcccaccatctgcaatgagatatggtcgaacttcatccaatacagtttcagacgttctcttcagttagtggtaattccactgctgaatctggagtagcaac

SEQ ID NO: 290 - DNA, 102 bp, Artificial Sequence
  Synthetic
atcattgaagcaagaagttgtcaaaacaattcatcattttaaagcttctgtcgatcaaattgatcattttattattcttttatttatatcaatagaaatata

SEQ ID NO: 291 - DNA, 102 bp, Artificial Sequence
  Synthetic
tttttgtgtatctttctagttattaaatgcatttcattgtttttctcaaaatgtaaatcgattctaaatgttgggttatttctataggtcggtgctcttgaa

SEQ ID NO: 292 - DNA, 102 bp, Artificial Sequence
  Synthetic
ttagcatttctctaacatttttcctttgataaggagaggaattaggaaatctaaccatctaactaggatattaactttactttagcaaatattatgttatgt

SEQ ID NO: 293 - DNA, 102 bp, Artificial Sequence
  Synthetic
ggcgagaaagctttcgagaaatatggaaagggcttcatctcaatgcattttctcagatcaacacttaggcacaaccaagaagatgctgctgttcaagtttgc

SEQ ID NO: 294 - DNA, 102 bp, Artificial Sequence
  Synthetic
atccaaattcttcaacaaatacccatctttcttctccatcttccaaccaagatccaggtcttcctccacgtgtcaaactcacaagacaagctctctcaatcc

SEQ ID NO: 295 - DNA, 102 bp, Artificial Sequence
  Synthetic
caacctcagggtagtgcaacatgtccaaggccgcaacaacaacctttacaagcagactcagcaatctcctaatgtgcaaggaagcgcctcccaatctcctct

SEQ ID NO: 296 - DNA, 102 bp, Artificial Sequence
  Synthetic
ctaaaaactattctcaatagtagtatgataccgagaagggtgctaggcactcaataataccctttagcatttctcaataataatactaaaaaaatattaaaa

SEQ ID NO: 297 - DNA, 102 bp, Artificial Sequence
  Synthetic
gacatatgaggttccctttcgagtacagcaattacatccttgattgttatagttactacggggcacctttgtttgaggcgagacgccttggcttcttccgaa

SEQ ID NO: 298 - DNA, 102 bp, Artificial Sequence
  Synthetic
cggctatcctccttgggtacagaaaagagctgttcttcctcctcctataatggtgtgaaacatcataagatcaaatcattgcataattaactagtagactac

SEQ ID NO: 299 - DNA, 102 bp, Artificial Sequence
  Synthetic
ggaagaaagtgaatgtgggggtgccatgcttgtggatgatgaaggaacaacacccagcagaagttggaaaacccattcttgatggctgcttctctaaagact

SEQ ID NO: 300 - DNA, 102 bp, Artificial Sequence
  Synthetic
tgcaatgtggaattatgtaaagattttaaggctcgtttggtacgatatattctggtcgtattatatttttatataatactatgttaaattttaattataagc

SEQ ID NO: 301 - DNA, 102 bp, Artificial Sequence
  Synthetic
agaatgagtatatgattcctttcaatgacttcactcttccctttccttaagcgaatccaccttctccgatatggcatctgaaacaagcccgagcatcatcgc

SEQ ID NO: 302 - DNA, 102 bp, Artificial Sequence
  Synthetic
aaattcagggggcatgatttagtacatgtcaaagttcggagagaaaaattgactaattagcctaaatataactcttcacctcataacacattttgactcact

SEQ ID NO: 303 - DNA, 102 bp, Artificial Sequence
  Synthetic
agatcggttctggcggcggagaagggttgagttgtagtgggcctccggttgaggcccatcaccggcaatggttttggaccttttcggaaccggtgggctgtc

SEQ ID NO: 304 - DNA, 102 bp, Artificial Sequence
  Synthetic
agggtgtgataaaacttgttcacaaacctaaattgtactagggaatcagcatggtgcccacaacataattttctccacaagttcacttggcaaatcataaaa

SEQ ID NO: 305 - DNA, 102 bp, Artificial Sequence
  Synthetic
cttgtgacgggcctcgtggatgggcctatcatttgggctgaaaatgaggtgccacatgtagaggcccaacctcgtgttaatgatggtagagaaacttacaaa

SEQ ID NO: 306 - DNA, 102 bp, Artificial Sequence
  Synthetic
ctttagagtacatatacgacatgtcgttttcttgggtaaacattttacgctcttttagccctttagagtatatatacgacaagtcgttttcatggataaatg

SEQ ID NO: 307 - DNA, 102 bp, Artificial Sequence
  Synthetic
aatttttcaattttgttttagctaaaccacacaaagctttgtcagtattaactattttagtttaatgttaaaaacaactgatgataacaaattgttttttca

SEQ ID NO: 308 - DNA, 102 bp, Artificial Sequence
  Synthetic
ttaggcctttggtagttatgttgaccaagcatttggtgaaatagaagtaacggcatcattggcttccccatgttgagcatacccaccaatcatgactgtcca

SEQ ID NO: 309 - DNA, 102 bp, Artificial Sequence
  Synthetic
aatgagattggggaagatccactaagtttaagtaatcggttctgccaagagatacctttctgacatggatgctcttcagtgtctccttcctacccatcagcc

SEQ ID NO: 310 - DNA, 102 bp, Artificial Sequence
  Synthetic
aaggtatatatagcctgattgattgaagatgagttgaaagccactgtttactagtgttgctagaccaataagggaaattttgagtgtcaagtgggaaagttc

SEQ ID NO: 311 - DNA, 102 bp, Artificial Sequence
  Synthetic
gagaggctggttttaacggcgggatatgctgaagcagaaagagcagttgctgttggaatttggagatgctgaagaggctaagcttctccttgctgagaaaga

SEQ ID NO: 312 - DNA, 102 bp, Artificial Sequence
  Synthetic
taataatgtatttatatatattgtttatttttcctaattttaagatagaagtacacacaatgttacaaatattatacatttaagctataaaatacaaattat

SEQ ID NO: 313 - DNA, 102 bp, Artificial Sequence
  Synthetic
gagttgttagtgatttttatcatagttattaaattatacatatatgaaatcgtaacattgtttatatacatgtatcatgatcaggcaatctatatgttttga

SEQ ID NO: 314 - DNA, 102 bp, Artificial Sequence
  Synthetic
gactttggattagcaaagctattgttgattaaccagagtcatactaacactagccattagagggacaaagggatatgtggcaccagaatggtttagcaacat

SEQ ID NO: 315 - DNA, 102 bp, Artificial Sequence
  Synthetic
ctgacaggtctcaatataattataatttatgcatatgtttgacatgatgatccatccagtagtatgtaagtctctataattaatgtttcattacttaataat

SEQ ID NO: 316 - DNA, 102 bp, Artificial Sequence
  Synthetic
tagataattgttaaatcacatattggagaaaattctagtagcatatttgaagtaagacactcaagttctctgattggatttagttattattatgattcatca

SEQ ID NO: 317 - DNA, 102 bp, Artificial Sequence
  Synthetic
tgaagtgaaacgtcgtttgagttcatcaaacctgtgtttacaggtatctgagagtccatggggattcagttcggttggagagttccatggcgatggagtccc

SEQ ID NO: 318 - DNA, 102 bp, Artificial Sequence
  Synthetic
aatcacaaaatacaaagcacaaaaattgtcttatgaaaaaagagtgataaaggtaccagccgagatcgtgttgactctaactcggtgttttctacccgcttc

SEQ ID NO: 319 - DNA, 102 bp, Artificial Sequence
  Synthetic
ttgcaccaaatcttccaacaagattgtctcttgccctaacaccgtcgatttcttggtgattcaacgcgcttattataactataccgagcaacatctgatctt

SEQ ID NO: 320 - DNA, 102 bp, Artificial Sequence
  Synthetic
tatggtagggcaatatctggttttcccacattctttctatctttcctttggtctagaaaatctcttatctaatttgtcttaacacacaatatttcaagcatc

SEQ ID NO: 321 - DNA, 102 bp, Artificial Sequence
  Synthetic
tttgcagctaagcacctcctcaataccaaaaaccaatcttctatgtccttacctttgtttcaaagacccttccccccatgacgatcatcgcttgatcacata

SEQ ID NO: 322 - DNA, 102 bp, Artificial Sequence
  Synthetic
tataaatcttcaactgcatacttacataaaagagtttaatgagtccaaactcgtaagaaggaataaaatttcaaaagaccaccaaaagagcagagttcttga

SEQ ID NO: 323 - DNA, 102 bp, Artificial Sequence
  Synthetic
ctaaattaattctctcattagtgatctactatggcaacttatgtctaactgtaattctctcattagtgattaaatagagtggtaacttgtattggatctgtt

SEQ ID NO: 324 - DNA, 102 bp, Artificial Sequence
  Synthetic
agaattccattcaagttacacgggcttccagtcacttcagttttccacccctactcgttctattttcttcattgcaacaaaaaagaatgttcgtgtttatga

SEQ ID NO: 325 - DNA, 102 bp, Artificial Sequence
  Synthetic
atgattcactacttgctcctgcacagatgaattcattgattgtggctgtgtcattgttctggtcttgttgaatttgaaagtttgcactttcatctaaagtag

SEQ ID NO: 326 - DNA, 102 bp, Artificial Sequence
  Synthetic
tggttcttgatggagagttcatttccatggatttgaggatcacattcacacttcatttccaaatccaaataagctcattttttgttgccttccaaaaagacc

SEQ ID NO: 327 - DNA, 102 bp, Artificial Sequence
  Synthetic
attatacagacggacagtttcctactgacagtagagaatttgcaaaacgcgatattttacacagtttatatgggaacagaaaacaggtggacacgctcaaga

SEQ ID NO: 328 - DNA, 102 bp, Artificial Sequence
  Synthetic
acctctaatgagacggacaaatgactctttggtgatttcttccctctgaacaatacaaaaaccaataagaaatagtaaaagaaaactaataatacaaatgag

SEQ ID NO: 329 - DNA, 24 bp, Artificial Sequence
  Synthetic
ccatatttaccgaatgtatcgctc

SEQ ID NO: 330 - DNA, 27 bp, Artificial Sequence
  Synthetic
ttagacagacaaatttacaatagtcta

SEQ ID NO: 331 - DNA, 25 bp, Artificial Sequence
  Synthetic
cttacaatgccacattattagagaa

SEQ ID NO: 332 - DNA, 25 bp, Artificial Sequence
  Synthetic
atgatacttggtctgtaatcaatga

SEQ ID NO: 333 - DNA, 26 bp, Artificial Sequence
  Synthetic
tttacaacttccataactgtcatgtc

SEQ ID NO: 334 - DNA, 27 bp, Artificial Sequence
  Synthetic
cttagaagggtactctctactcaaaca

SEQ ID NO: 335 - DNA, 25 bp, Artificial Sequence
  Synthetic
tgttattctaatgaaatctgatccg

SEQ ID NO: 336 - DNA, 25 bp, Artificial Sequence
  Synthetic
gaactttagtagcaatgtgagttgg

SEQ ID NO: 337 - DNA, 43 bp, Artificial Sequence
  Synthetic
gaaggtgaccaagttcatgcttgaaaatgaggttgcccagagt

SEQ ID NO: 338 - DNA, 43 bp, Artificial Sequence
  Synthetic
gaaggtcggagtcaacggatttgaaaatgaggttgcccagagc

SEQ ID NO: 339 - DNA, 20 bp, Artificial Sequence
  Synthetic
ctctcttggcatgcagcaca

SEQ ID NO: 340 - DNA, 44 bp, Artificial Sequence
  Synthetic
gaaggtgaccaagttcatgctcgagtcttttcacacctttgctc

SEQ ID NO: 341 - DNA, 44 bp, Artificial Sequence
  Synthetic
gaaggtcggagtcaacggattcgagtcttttcacacctttgcta

SEQ ID NO: 342 - DNA, 21 bp, Artificial Sequence
  Synthetic
ttgtctgagccactcaaggca

SEQ ID NO: 343 - DNA, 45 bp, Artificial Sequence
  Synthetic
gaaggtgaccaagttcatgctccattcgtgtcaacattccaaaca

SEQ ID NO: 344 - DNA, 45 bp, Artificial Sequence
  Synthetic
gaaggtcggagtcaacggattccattcgtgtcaacattccaaacc

SEQ ID NO: 345 - DNA, 24 bp, Artificial Sequence
  Synthetic
gaaagcctggttgaacatgagtct

SEQ ID NO: 346 - DNA, 47 bp, Artificial Sequence
  Synthetic
gaaggtgaccaagttcatgctacaggaaaagtattggaatgcaatgt

SEQ ID NO: 347 - DNA, 47 bp, Artificial Sequence
  Synthetic
gaaggtcggagtcaacggattacaggaaaagtattggaatgcaatga

SEQ ID NO: 348 - DNA, 29 bp, Artificial Sequence
  Synthetic
gctttagtattaagtttgttgaagggact

SEQ ID NO: 349 - DNA, 43 bp, Artificial Sequence
  Synthetic
gaaggtgaccaagttcatgctgcaagcaaccaccaaaactgat

SEQ ID NO: 350 - DNA, 43 bp, Artificial Sequence
  Synthetic
gaaggtcggagtcaacggattgcaagcaaccaccaaaactgaa

SEQ ID NO: 351 - DNA, 21 bp, Artificial Sequence
  Synthetic
gcttgcgctgcaattcggtat

SEQ ID NO: 352 - DNA, 46 bp, Artificial Sequence
  Synthetic
gaaggtgaccaagttcatgctcctgttaaatccatgctgcatagag

SEQ ID NO: 353 - DNA, 46 bp, Artificial Sequence
  Synthetic
gaaggtcggagtcaacggattcctgttaaatccatgctgcatagaa

SEQ ID NO: 354 - DNA, 20 bp, Artificial Sequence
  Synthetic
gtcaactgtggtaaccccca

(19) United States
(12) Patent Application Publication
(10) Pub. No.: US 2025/0268163 A1
(43) Pub. Date: Aug. 28, 2025

(54) CANNABIS PLANTS EXHIBITING POWDERY MILDEW RESISTANCE AND METHODS FOR OBTAINING SAME

(71) Applicant: CANOPY GROWTH CORPORATION, Smiths Falls (CA)

(72) Inventor: Amit DEOKAR, Smiths Falls (CA)

(21) Appl. No.: 18/282,256

(22) PCT Filed: May 6, 2022
(86) PCT No.: PCT/CA2022/050716

§ 371 Date: Sep. 15, 2023

Related U.S. Application Data
(60) Provisional application No. 63/185,752, filed on May 7, 2021.
(60) Provisional application No. 63/304,169, filed on Jan. 28, 2022.

(51) Int. Cl. A01H 1/04; A01H 1/00; C12Q 1/6895

(52) U.S. Cl. CPC A01H 1/045; A01H 1/1255; C12Q 1/6895; C12Q 2600/13; C12Q 2600/156

(57) ABSTRACT

The present technology is in the field of plant breeding and genetics, particularly as it pertains to the genus Cannabis. More specifically, there are provided methods and compositions for producing a population of Cannabis plants with enhanced resistance to powdery mildew. The methods use the detection of molecular genetic markers linked to powdery mildew resistance loci to select for plants displaying an enhanced powdery mildew resistance phenotype.

### CROSS-REFERENCE TO RELATED APPLICATIONS

This application is a National Phase Entry of International PCT patent application no. PCT/CA2022/050716 filed on May 6, 2022, which claims the benefit of and priority to U.S. provisional patent application No. 63,185,752, filed on May 7, 2021; and to U.S. provisional patent application No. 63/304,169, filed on Jan. 28, 2022; the content of each of which is herein incorporated in entirety by reference.

### SEQUENCE LISTING

This application contains a Sequence Listing which has been submitted in ASCII format via Patent Center and is hereby incorporated by reference in its entirety. The ASCII copy, created on Apr. 4, 2024, is named “100728-185 Updated Sequence Listing.txt” and is 90 KB.

### FIELD OF TECHNOLOGY

The present technology generally relates to Cannabis plants exhibiting powdery mildew resistance as well as molecular markers for same and methods of producing same.

### BACKGROUND

Cannabis sativa L. is a diploid (2n=20) annual species belonging to the genus Cannabis in the Cannabaceae family. Cannabis sativa is commonly known as cannabis, hemp, Indian hemp, marihuana, and marijuana. Cannabis has been cultivated throughout the centuries as a source of fiber and food, and for its therapeutic and recreational properties (Farnsworth, N. R., 1969). The pharmaceutical and recreational properties of this unique plant are associated with a unique class of terpenophenolic compounds known as cannabinoids. Cannabinoids interact with receptors of human and animal endocannabinoid systems and can lead to a plethora of potential medical and therapeutic effects (Di Marzo & Piscitelli, 2015). Cannabinoids are produced and stored in the glandular trichomes present on the surfaces of female inflorescences. Over 500 phytocannabinoids and non-cannabinoid constituents have been identified and/or isolated from C. sativa L, including the well-known psychoactive compound Δ9-tetrahydro-cannabinol (Δ9-THC) and non-psychoactive compounds such as cannabidiol (CBD) (an isomer of THC), cannabichromene (CBC) and cannabigerol (CBG) (ElSohly et al., 2017).

As the legal cannabis market size continues to grow, a consistent supply of high-quality cannabis products remains a priority. Of the many factors affect the yield, quality, and marketability of cannabis products, the levels of infection by destructive pathogens such as powdery mildew and contamination by agrochemicals used to control diseases are among the most important. General susceptibility of the commercial material, the pathogenicity of the fungus, large seasonal spore loads, and the lack of effective and approved disease management strategies make powdery mildew a major concern for commercial cannabis cultivation.

Cannabis powdery mildew is caused by biotrophic parasitic fungi such as the Golovinomyces species (Thompson et al., 2017; P6pin et al., 2018). Powdery mildew develops on leaves, stems, and flower buds at any stage of development. Early stages of infection can be observed on young leaves as white mycelium on the leaf surface, whereas in advanced stages of infection, profuse sporulation results in a powdery appearance on the leaf surface (Punja et al., 2019). Powdery mildew infection can severely damage a plant by limiting photosynthetic activities and reducing nutrient availability to the plant, causing premature leaf fall, and reducing overall vigor and potential yield (Scott and Punja, 2020).

Powdery mildew disease is largely managed by applications of chemical products or bio-control agents. However, development and growth of resistant cultivars will be a more sustainable, effective, and economical strategy against this disease. Similar approaches have been used successfully for a wide range of crops such as wheat, barley, and tomato (Ren et al., 2017; Bengtsson et al., 2017; Nekrasov et al., 2017). One of the major hurdles for using this approach in cannabis is a lack of natural resistance against the powdery mildew in cannabis, as well as a lack of knowledge of the genetic mechanisms for regulating powdery mildew disease resistance in cannabis.

Both qualitative and quantitative resistance against the powdery mildew disease has been reported in many plant species. In some plants, such as tomato nine monogenic resistance genes (Ol-genes) have been identified confirming resistance to Tomato powdery mildew (Seifi et al 2014), while in other species, such in wheat over 100 powdery mildew resistance quantitative trait loci (QTL) have been identified (Ren et al 2017). Resistance to powdery mildew in Hop (Humulus lupulus L. var lupulus) another member of the Cannabaceae plant family, has been reported as controlled by both qualitative and quantitative genetic control (Henning et al. 2017). Recently, Padgitt-Cobb and others (2020) identified a narrow genomic region mapped to hop linkage group 10. This region contained several putative R-genes and putative peroxidase-3 genes as a potential candidate for genes involved in resistance for powdery mildew in the hop.

Attempts to breed powdery mildew resistant cannabis cultivars have been greatly impeded by the lack of a natural source of resistance and a poor understanding of the inheritance of disease resistance, as well as the unavailability of robust genetic markers linked to the trait. The use of markers in cannabis breeding could not only reduce the cost of developing new varieties but may also increase the precision and efficiency of selection of powdery mildew resistant breeding lines in the breeding program, as well as reduce the number of years required to develop new and improved resistant varieties.

There is a need to identify genetic markers in Cannabis for powdery mildew resistance and to develop Cannabis strains exhibiting such resistance.

### SUMMARY

According to various aspects, the present technology relates to a Cannabis plant, plant part, tissue or cell thereof, wherein the Cannabis plant, plant part, tissue or cell thereof has an enhanced resistance to powdery mildew, as well as molecular markers for same and methods of producing same.

According to various aspects, the present disclosure provides a method for creating a population of Cannabis plants with enhanced powdery mildew resistance, the method comprising: providing a first population of Cannabis plants; detecting the presence of a genetic marker that is genetically linked to a powdery mildew resistance locus in a defined linkage group, as described herein, by about 20 cM or less in the first population; selecting one or more Cannabis plants containing said marker from the first population of Cannabis plants; and producing a population of offspring from at least one of said selected Cannabis plants.

The present disclosure further provides a method wherein the genetic marker detected is genetically linked to the powdery mildew resistance locus on the defined linkage group by less than about 15 cM, or less than about 10 cM, or less than about 5 cM.

In some embodiments of the present technology, the powdery mildew resistance locus is located within a chromosome interval on Chromosome 2.

In some embodiments of the present technology, the powdery mildew resistance locus is located within a chromosome interval on Chromosome 2 at 83660977-84353662 bp.

In some embodiments of the present technology, the genetic marker for powdery mildew resistance is located within a chromosome interval on Chromosome 2.

In some embodiments of the present technology, the genetic marker for powdery mildew resistance is located within a chromosome interval on Chromosome 2 at 83660977-84353662 bp.

In some embodiments of the present technology, the genetic marker is selected from the markers set forth in FIGS. 4A-4E.

In some embodiments of the present technology, the genetic marker has a sequence selected from the sequences set forth in FIGS. 5A-5M. In some embodiments, the genetic marker has a sequence selected from sequences having at least, greater than or about 75%, about 80%, about 85%, about 90%, about 95%, about 96%, about 97%, about 98% or about 99% sequence identity to sequences set forth in FIGS. 5A-5M.

In another embodiment, the present disclosure provides detecting a genetic marker located within a chromosome interval comprising and flanked by a first genetic marker and a second genetic marker, as described herein. In another embodiment, the present disclosure provides detecting a genetic marker located within a chromosome interval comprising and/or flanked by one or more genetic marker as described herein, e.g., by a DNA sequence comprising or consisting of the genetic marker. In some embodiments of the present technology, the powdery mildew resistance chromosome interval is located on Chromosome 2.

In some embodiments of the present technology, the powdery mildew resistance chromosome interval is located on Chromosome 2 at 83660977-84353662 bp.

In some embodiments of the present technology, the genetic marker is selected from the markers set forth in FIGS. 4A-4E.

In some embodiments of the present technology, the genetic marker has a sequence selected from the sequences set forth in FIGS. 5A-5M. In some embodiments, the genetic marker has a sequence selected from sequences having at least, greater than or about 75%, about 80%, about 85%, about 90%, about 95%, about 96%, about 97%, about 98% or about 99% sequence identity to sequences set forth in FIGS. 5A-5M.

The present disclosure also provides a method of creating a population of Cannabis plants comprising at least one allele associated with enhanced powdery mildew resistance comprising at least one sequence selected from the group of genetic markers described herein, the method comprising the steps of: genotyping a first population of Cannabis plants, said population containing at least one allele associated with enhanced powdery mildew resistance, the at least one allele associated with enhanced powdery mildew resistance comprising at least one sequence selected from the group consisting of sequences of genetic markers described herein; selecting from said first population one or more identified Cannabis plants containing said at least one allele associated with enhanced powdery mildew resistance comprising at least one sequence selected from the group consisting of sequences of genetic markers described herein; and producing from said selected Cannabis plants a second population, thereby creating a population of Cannabis plants comprising at least one allele associated with enhanced powdery mildew resistance comprising at least one sequence selected from the group consisting of sequences of genetic markers described herein.

The present disclosure further provides a method for creating a population of Cannabis plants with enhanced powdery mildew resistance comprising: providing a first population of Cannabis plants; concurrently detecting the presence of at least one genetic marker that is genetically linked to one or more of the genetic markers for powdery mildew resistance described herein by about 20 cM or less in the first population; selecting one or more Cannabis plants containing said at least one markers from the first population of Cannabis plants; and producing a population of offspring from at least one of said selected Cannabis plants. In one embodiment, the at least one genetic marker detected is genetically linked to at least one of the genetic markers for powdery mildew resistance described herein by less than about 15 cM. In another embodiment, the at least one genetic marker detected is genetically linked to at least one of the genetic markers for powdery mildew resistance described herein by less than about 10 cM. In another embodiment, the at least one genetic marker detected is genetically linked to at least one of the genetic markers for powdery mildew resistance described herein by less than about 5 cM.

According to various aspects, the present disclosure provides a population of Cannabis plants with enhanced powdery mildew resistance.

According to various aspects, the present disclosure provides a population of Cannabis plants with one or more genetic markers for powdery mildew resistance described herein.

According to various aspects, the present disclosure provides a molecular marker, e.g., a genetic marker, for use in breeding and/or selecting Cannabis plants with enhanced powdery mildew resistance. In an embodiment, the molecular marker comprises or consists of a genetic marker as described herein, e.g., having the sequence thereof, or a portion of the sequence thereof. In an embodiment, the molecular marker comprises or consists of an isolated nucleic acid molecule having the sequence of a genetic marker as described herein, or a portion thereof, or a nucleotide sequence having at least, greater than or about 75% sequence identity thereto, or greater than or about 85% sequence identity to the complimentary sequence thereto.

According to various aspects, the present technology relates to an isolated nucleic acid molecule for identifying a genetic marker for powdery mildew resistance as described herein.

According to various aspects, the present technology relates to an isolated polypeptide encoded by the isolated nucleic acid molecule as described herein.

According to various aspects, the present technology relates to an antibody that specifically binds the isolated polypeptide described herein.

According to various aspects, the present technology relates to an organism, tissue or cell comprising the isolated nucleic molecule or the isolated polypeptide or the genetic marker as described herein. In some instances, the organism, tissue or cell is a plant, a plant tissue or a plant cell. In some further instances, the organism, tissue or cell is a Cannabis plant, a Cannabis tissue, or a Cannabis cell.

Other aspects and features of the present disclosure will become apparent to those ordinarily skilled in the art upon review of the following description of specific embodiments.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 shows results from a genome-wide scan (Chromosomes 1-9) for each of 4 related pedigrees (P1, P2, P3, P4) sharing the same powdery mildew resistance/susceptibility phenotype.

FIG. 2 is a chart of LOD score vs. Mbp, which shows an enlarged view of a QTL region identified on Chromosome 2 for each of the 4 pedigrees (P1, P2, P3, P4).

FIG. 3 is a chart of phenotype (AUDPC) vs. genotype (Phase A or B on P5) at the QTL for each of the 4 pedigrees (P1, P2, P3, P4).

FIGS. 4A-4E show SNP positions and alleles for the two phases (Phase A and Phase B) for powdery mildew resistance genetic markers in the QTL region on Chromosome 2.

FIGS. 5A-5M shows the sequences of powdery mildew resistance genetic markers in the QTL region on Chromosome 2; sequences are shown in the 5′ to 3′ orientation from left to right, with the Phase A and Phase B alleles shown in square brackets in the center of the sequence as [A/B].

FIG. 6 is a graph showing the results of allele specific T-ARMS for the 91K.chr2Ap83851294[T/C] SNP. Agarose gel analysis of 91K.chr2Ap83851294[T/C] SNP marker. The 203 and 150 bp amplicons corresponds to C and T allele of the 91K.chr2Ap83851294 SNP. All the PCR reactions except the no template control (NTC) showed a 302 bp PCR control amplicon. The resistant parent (in duplicate 1 and 2) and seven F₁ plants shows heterozygous genotype (both 203 and 150 bp amplicon corresponding to C and T allele), while the susceptible parent (in duplicate 1 and 2) and seven F₁ shows homozygous CC genotype corresponds to a 203 bp amplicon.

FIG. 7 is a graph illustrating representative result (allele description plot) of KASP/PACE genotype assay for SNP marker 91kv2Chr2Ap84275859. Heterozygous individuals with allele 1/2 are represented in green, while the homozygous individual with allele 1 are represented in red

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure in which variations and additions do not depart from the present technology. Hence, the following description is intended to illustrate some particular embodiments of the technology, and not to exhaustively specify all permutations, combinations and variations thereof.

### Definitions

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not. Every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species (McPartland & Guy, 2017). For example some describe Cannabis plants as a single species, C. sativa L., with multiple subspecies (Small & Cronquist, 1976)(McPartland & Small, 2020) while others classify Cannabis plants into multiple species, most commonly as C. sativa L. and C. indica Lam. and sometimes additionally as C. ruderalis Janisch. (Schultes et al., 1974), depending on multiple criteria including morphology, geographic origin, chemical content, and genetic measurements. Regardless, all plants of genus Cannabis can interbreed and produce fertile offspring (Small, 1972).

As used herein, the expression “powdery mildew” refers to a disease which affects a wide range of plants and is characterized by development of white mycelium or a powdery appearance on the leaf surface. Powdery mildew (also referred to as “PM”) can be caused by many different fungal species in the genera Erysiphe, Microsphaera, Phyllactinia, Podosphaera, Sphaerotheca, and Uncinula (Braun and Cook, 2012). At present, there is no consensus on the species and races of powdery mildew pathogen affecting the Cannabis plant. Powdery mildew on cannabis has been reported to be caused by Podosphaera macularis (formerly Sphaerotheca macularis), Leveillula taurica (McPartland 1996), and Golovinomyces species including G. cichoracearum (P6pin et al 2018), G. ambrosiae (Wiseman et al., 2021) and G. spadiceus (Farinas and Peduto Hand 2020).

The term “strain” as used herein refers to different varieties of the plant genus Cannabis. For example, the term “strain” can refer to different pure or hybrid varieties of Cannabis plants. In some instances, the Cannabis strain of the present technology can by a hybrid of two strains. Different Cannabis strains often exhibit distinct chemical compositions with characteristic levels of cannabinoids and terpenes, as well as other components. Differing cannabinoid and terpene profiles associated with different Cannabis strains can be useful e.g. for the treatment of different diseases, or for treating different subjects with the same disease.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In another embodiment, a cannabinoid is one of a class of diverse chemical compounds that may act on cannabinoid receptors such as CB₁ and CB₂ in cells that alter neurotransmitter release in the brain.

In many cases, a cannabinoid can be identified because its chemical name will include the text string “*cannabi*”. However, there are a number of cannabinoids that do not use this nomenclature, such as for example those described herein.

As used herein, the expression “% by weight” is calculated based on dry weight of the total material.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids. As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). Further, in some embodiments of the present disclosure, the cannabinoid is a cannabinoid dimer. The cannabinoid may be a dimer of the same cannabinoid (e.g. THC-THC) or different cannabinoids. In an embodiment of the present disclosure, the cannabinoid may be a dimer of THC, including for example Cannabisol.

In an embodiment, a cannabinoid may occur in its free form, or in the form of a salt; an acid addition salt of an ester; an amide; an enantiomer; an isomer; a tautomer; a prodrug; a derivative of an active agent of the present technology; different isomeric forms (for example, enantiomers and diastereoisomers), both in pure form and in admixture, including racemic mixtures; enol forms.

As used herein, the expressions “nucleic acid,” “nucleic acid molecule,” “oligonucleotide,” and “polynucleotide” are each used herein to refer to a polymer of at least three nucleotides. In some embodiments, a nucleic acid comprises deoxyribonucleic acid (DNA). In some embodiments, a nucleic acid comprises ribonucleic acid (RNA). In some embodiments, a nucleic acid is single stranded. In some embodiments, a nucleic acid is double stranded. In some embodiments, a nucleic acid comprises both single and double stranded portions. Unless otherwise stated, the terms encompass nucleic acid-like structures with synthetic backbones, as well as amplification products. In some embodiments, nucleic acids of the present disclosure are linear nucleic acids.

As used herein, the term “gene” refers to a part of the genome that codes for a product (e.g., an RNA product and/or a polypeptide product). A “gene sequence” is a sequence that includes at least a portion of a gene (e.g., all or part of a gene) and/or regulatory elements associated with a gene. In some embodiments, a gene includes coding sequence; in some embodiments, a gene includes non-coding sequence. In some particular embodiments, a gene may include both coding (e.g., exonic) and non-coding (e.g., intronic) sequences. In some embodiments, a gene may include one or more regulatory elements (e.g., a promoter) that, for example, may control or impact one or more aspects of gene expression (e.g., cell-type-specific expression, inducible expression, etc.).

As used herein, the expression “coding sequence” refers to a sequence of a nucleic acid or its complement, or a part thereof, that: i) can be transcribed to an mRNA sequence that can be translated to produce a polypeptide or a fragment thereof; or ii) an mRNA sequence that can be translated to produce a polypeptide or a fragment thereof. Coding sequences include exons in genomic DNA or immature primary RNA transcripts, which are joined together by the cell's biochemical machinery to provide a mature mRNA.

As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence, or the increase or reduction/elimination of an existing character, property, function, phenotype or trait not found in the protein encoded by the parental sequence.

The expression “degree or percentage of sequence identity” refers herein to the degree or percentage of sequence identity between two sequences after optimal alignment. Percentage of sequence identity (or degree of identity) is determined by comparing two aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

As used herein, the term “isolated” refers to nucleic acids or polypeptides that have been separated from their native environment, including but not limited to virus, proteins, glycoproteins, peptide derivatives or fragments or polynucleotides. For example, the expression “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5′ and 3′ ends of the nucleic acid) from which the nucleic acid is derived.

Two nucleotide sequences or amino-acids are said to be “identical” if the sequence of nucleotide residues or amino-acids in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Needleman and Wunsch, J. Mol. Biol. 48: 443 (1970), by the search for similarity method of Pearson and Lipman, Proc. Natl. Acad. Sci. (U.S.A.) 85: 2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection. Other alignment programs may also be used such as: “Multiple sequence alignment with hierarchical clustering”, F. CORPET, 1988, Nucl. Acids Res., 16 (22), 10881-10890.

As used herein, the expression “conservative substitutions” refers to a substitution made in an amino acid sequence of a polypeptide without disrupting the structure or function of the polypeptide. Conservative amino acid substitutions may be accomplished by substituting amino acids with similar hydrophobicity, polarity, and R-chain length for one another. Additionally, by comparing aligned sequences of homologous proteins from different species, conservative amino acid substitutions may be identified by locating amino acid residues that have been mutated between species without altering the basic functions of the encoded proteins. Amino acid substitutions that are conservative are typically as follows: i) hydrophilic: Alanine (Ala) (A), Proline (Pro) (P), Glycine (Gly) (G), Glutamic acid (Glu) (E), Aspartic acid (Asp) (D), Glutamine (Gln) (Q), Asparagine (Asn) (N), Serine (Ser) (S), Threonine (Thr) (T); ii) Sulphydryl: Cysteine (Cys) (C); iii) Aliphatic: Valine (Val) (V), Isoleucine (Ile) (I), Leucine (Leu) (L), Methionine (Met) (M); iv) Basic: Lysine (Lys) (K), Arginine (Arg) (R), Histidine (His) (H); and v) Aromatic: Phenylalanine (Phe) (F), Tyrosine (Tyr) (Y), Tryptophan (Trp) (W).

An “expression system” as used herein refers to reagents and components (e.g. in a kit) and/or solutions comprising said reagents and components for recombinant protein expression, wherein the expression system is cell free and includes optionally translation competent extracts of whole cells and/or other translation machinery reagents or components optionally in a solution, said reagents and components optionally including RNA polymerase, one or more regulatory protein factors, one or more transcription factors, ribosomes, and tRNA, optionally supplemented with cofactors and nucleotides, and the specific gene template of interest. Chemical based expression systems are also included, optionally using unnaturally occurring amino acids. In some instances, the expression systems of the present technology are in vitro expression systems.

The expressions “transformed with”, “transfected with”, “transformation” and “transfection” are intended to encompass introduction of nucleic acid (e.g. a construct) into a cell by one of many possible techniques known in the art.

The term “primer” as used herein, typically refers to oligonucleotides that hybridize in a sequence specific manner to a complementary nucleic acid molecule (e.g., a nucleic acid molecule comprising a target sequence). In some embodiments, a primer will comprise a region of nucleotide sequence that hybridizes to at least 8, e.g., at least 10, at least 15, at least 20, at least 25, or 20 to 60 nucleotides of a target nucleic acid (i.e., will hybridize to a sequence of the target nucleic acid). In general, a primer sequence is identified as being either “complementary” (i.e., complementary to the coding or sense strand (+)), or “reverse complementary” (i.e., complementary to the anti-sense strand (−)). In some embodiments, the term “primer” may refer to an oligonucleotide that acts as a point of initiation of a template-directed synthesis using methods such as PCR (polymerase chain reaction) under appropriate conditions (e.g., in the presence of four different nucleotide triphosphates and a polymerization agent, such as DNA polymerase in an appropriate buffer solution containing any necessary reagents and at suitable temperature(s)). Such a template directed synthesis is also called “primer extension.” For example, a primer pair may be designed to amplify a region of DNA using PCR. Such a pair will include a “forward primer” and a “reverse primer” that hybridize to complementary strands of a DNA molecule and that delimit a region to be synthesized and/or amplified.

As used herein, the expression “wild-type” refers to a typical or common form existing in nature; in some embodiments it is the most common form.

As used herein, “allele” generally refers to an alternative nucleic acid sequence at a particular locus; the length of an allele can be as small as 1 nucleotide base but is typically larger. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease prone plants. A favorable allelic form of a chromosome interval is a chromosome interval that includes a nucleotide sequence that contributes to superior agronomic performance at one or more genetic loci physically located on the chromosome interval.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

“Crossed” or “cross” means to produce progeny via fertilization (e.g. cells, seeds or plants) and includes crosses between plants (sexual) and self fertilization (selfing).

“Gene” refers to a heritable sequence of DNA, i.e., a genomic sequence, with functional significance. The term “gene” can also be used to refer to, e.g., a cDNA and/or a mRNA encoded by a genomic sequence, as well as to that genomic sequence.

“Genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome interval. The terms “phenotype,” or “phenotypic trait” or “trait” refers to one or more trait of an organism, i.e., the detectable characteristics of a cell or organism which can be influenced by genotype. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of several genes.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells that can be cultured into a whole plant.

“Plant” refers to a whole plant or any part thereof, such as a cell or tissue culture derived from a plant, comprising any of: whole plants, plant components or organs (e.g., leaves, stems, roots, etc,), plant tissues, seeds, plant cells, and/or progeny of the same. A plant cell is a biological cell of a plant, taken from a plant or derived through culture from a cell taken from a plant. As used herein, the expression “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots.” Plant part may also include certain extracts such as kief or hash which includes Cannabis trichomes or glands.

“Polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

A “population of plants” or “plant population” means a set comprising any number, generally more than one, of individuals, objects, or data from which samples are taken for evaluation, e.g. estimating QTL effects. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silica representations of the plants. The population members need not be identical to the population members selected for use in subsequent cycles of analyses or those ultimately selected to obtain final progeny plants. Often, a plant population is derived from a single biparental cross, but may also derive from two or more crosses between the same or different parents. Although a population of plants may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

“Resistance allele” means the nucleic acid sequence associated with powdery mildew resistance or enhanced resistance to powdery mildew. Similarly, “resistance marker” or “resistance loci” means respectively the marker or loci associated with powdery mildew resistance or enhanced resistance to powdery mildew.

“Recombinant” in reference to a nucleic acid or polypeptide indicates that the material (e.g., a recombinant nucleic acid, gene, polynucleotide, polypeptide, etc.) has been altered by human intervention. The term recombinant can also refer to an organism that harbors recombinant material, e.g., a plant that comprises a recombinant nucleic acid is considered a recombinant plant.

“Resistance” or “enhanced resistance” in a plant to disease conditions (such as powdery mildew) is an indication that the plant is less affected by disease conditions with respect to yield, survivability and/or other relevant agronomic measures, compared to a less resistant, more “susceptible” plant. The terms “enhanced resistance” and “improved resistance” are used interchangeably. Resistance is a relative term, indicating that a “resistant” plant survives and/or produces better yields in disease conditions (e.g., powdery mildew conditions) compared to a different (less resistant) plant (e.g., a different Cannabis strain) grown in similar conditions. As used in the art, disease “resistance” is sometimes used interchangeably with disease “tolerance.” One of skill in the art will appreciate that plant resistance to disease conditions varies widely, and can represent a spectrum of more-resistant or less-resistant phenotypes. However, by simple observation, one of skill in the art can generally determine the relative resistance or susceptibility of different plants, plant lines or plant families under disease conditions, and furthermore, will also recognize the phenotypic gradations of “resistant.” In an embodiment, a Cannabis plant with enhanced resistance to powdery mildew is a plant having enhanced powdery mildew resistance compared to a wild-type Cannabis plant. In another embodiment, a Cannabis plant with enhanced resistance to powdery mildew is a plant having enhanced powdery mildew resistance compared to a different, less resistant or more susceptible plant (e.g., a different Cannabis strain) grown in similar conditions.

As used herein, the term “chromosome interval” designates a contiguous linear span of genomic DNA that resides in planta on a single chromosome. The term also designates any and all genomic intervals defined by any of the markers set forth in this disclosure. The genetic elements located on a single chromosome interval are physically linked and the size of a chromosome interval is not particularly limited. In some aspects, the genetic elements located within a single chromosome interval are genetically linked, typically with a genetic recombination distance of, for example, less than or equal to 20 cM, or alternatively, less than or equal to 15 cM, or alternatively, less than or equal to 10 cM, or alternatively, less than or equal to 5 cM. That is, two genetic elements within a single chromosome interval undergo meiotic recombination at a frequency of less than or equal to about 20%, or less than or equal to 15%, or less than or equal to 10%, or less than or equal to 5%, respectively.

The boundaries of a chromosome interval can be defined by genetic recombination distance or by markers. In one embodiment, the boundaries of a chromosome interval comprise markers. In another embodiment, the boundaries of a chromosome interval comprise markers that will be linked to the gene controlling the trait of interest, i.e., any marker that lies within a given interval, including the terminal markers that define the boundaries of the interval, and that can be used as a marker for the presence or absence of powdery mildew resistance. In one embodiment, the intervals described herein encompass marker clusters that co-segregate with powdery mildew resistance. The clustering of markers generally occurs in relatively small domains on the chromosomes, indicating the presence of a genetic locus controlling the trait of interest in those chromosome regions. The interval encompasses markers that map within the interval as well as the markers that define the terminal.

An interval described by the terminal markers that define the endpoints of the interval will include the terminal markers and any marker localizing within that chromosome domain, whether those markers are currently known or unknown. Although it is anticipated that one skilled in the art may describe additional polymorphic sites at marker loci in and around the markers identified herein, any marker within the chromosome intervals described herein that are associated with powdery mildew resistance fall within the scope of the present disclosure.

“Quantitative trait loci” or a “quantitative trait locus” (QTL) is a genetic domain that effects a phenotype that can be described in quantitative terms and can be assigned a “phenotypic value” which corresponds to a quantitative value for the phenotypic trait. A QTL can act through a single gene mechanism or by a polygenic mechanism. In some aspects, the disclosure provides QTL chromosome intervals, where a QTL (or multiple QTLs) that segregates with powdery mildew resistance is contained in those intervals. In one embodiment, the boundaries of chromosome intervals are drawn to encompass markers that will be linked to one or more QTL. In other words, the chromosome interval is drawn such that any marker that lies within that interval (including the terminal markers that define the boundaries of the interval) is genetically linked to the QTL. Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. Regardless, knowledge of how many QTL are in a particular interval is not necessary to make or practice the present technology.

Chromosome Intervals, QTLs and Molecular Markers Associated with Powdery Mildew Resistance

The present disclosure identifies QTLs and molecular markers linked to powdery mildew resistance. The present disclosure provides for strains with powdery mildew resistance. The present disclosure includes and provides for methods to introduce resistance into existing Cannabis strains as part of a powdery mildew management strategy. The present disclosure identifies loci associated with powdery mildew resistance, provides tightly linked single nucleotide polymorphisms (SNPs) and demonstrates that certain genetic markers are important for powdery mildew resistance.

As provided herein, powdery mildew resistance can be obtained using markers described herein, which tag certain genetic markers identified as linked to powdery mildew resistance. Such markers can be used in early generations to select for populations with resistance or used in later generations to characterize and prioritize which material to advance into yield testing trials. The markers provided herein also provide for the breeding of plants incorporating all known powdery mildew resistance alleles into a single strain. The present disclosure provides an expanded base of powdery mildew resistant material for breeding programs, and this larger base allows agronomically acceptable and high-yielding varieties with powdery mildew resistance.

In one embodiment, the present disclosure provides a plant comprising a nucleic acid molecule selected from the group consisting of sequences of genetic markers described herein and fragments thereof, and complements of both. In another embodiment, the present disclosure also provides a plant comprising the allele of at least one chromosome interval or genetic marker described herein, or fragments and complements thereof. The present disclosure also provides for any plant comprising any combination of one or more powdery mildew resistance loci linked to at least one marker selected from the group consisting of sequences of genetic markers described herein.

Locations in the Cannabis genome of QTLs and the chromosome intervals comprising markers closely linked to it are described herein. Genetic and physical map positions of markers and chromosome intervals associated with QTLs are described herein. In some embodiments, genetic map loci are represented in cM, with position zero being the first (most distal) marker known at the beginning of the chromosome on a Cannabis genetic map. As used herein, “cM” refers to the classical definition of a centimorgan (Edwards, J. H., 1919) wherein one cM is equal to a 1% chance that a trait at one genetic locus will be separated from a trait at another locus due to crossing over in a single meiosis (meaning the traits cosegregate 99% of the time), and this definition is used herein to delineate map locations pertaining to this technology.

Thus, one skilled in the art can use the present technology to improve the efficiency of breeding for improved powdery mildew resistance in Cannabis by associating resistance phenotypes with genotypes at previously unknown resistance loci in the cannabis genome. Disclosed herein are chromosome intervals that comprise alleles responsible for phenotypic differences between powdery mildew resistant and powdery mildew susceptible cannabis lines. Example chromosome intervals are characterized by the genomic regions including and flanked by and including the markers described herein and comprise markers within or closely linked to (e.g., within 20 cM of) the loci described herein. The present technology also comprises other intervals whose borders fall between, or any interval closely linked to those intervals. Examples of markers useful for this purpose comprise the SNP markers described herein, or any marker that maps within the chromosome intervals described herein (including the termini of the intervals), or any marker linked to those markers. Such markers can be assayed simultaneously or sequentially in a single sample or population of samples. Accordingly, the markers and methods of the present disclosure can be utilized to guide the breeding of cannabis varieties with the desired complement (set) of allelic forms of chromosome intervals associated with superior agronomic performance (powdery mildew resistance, along with any other available markers for yield, other disease resistance, etc.). Any of the disclosed marker alleles can be introduced into a cannabis line using standard methods such as, without limitation, via introgression, by traditional breeding (or introduced via transformation, or both) to yield a Cannabis plant with superior agronomic performance. The number of alleles associated with powdery mildew resistance that can be introduced or be present in a Cannabis plant of the present disclosure ranges from one to the number of alleles disclosed herein, each integer of which is incorporated herein as if explicitly recited.

In some embodiments, marker-assisted selection (MAS) using additional markers flanking either side of the QTLs described herein can provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein could be useful and within the scope of the present technology.

Similarly, by identifying plants lacking the desired marker locus, susceptible or less resistant plants can be identified, and, e.g., eliminated from subsequent crosses. Similarly, these marker loci can be introgressed into any desired genomic background, germplasm, plant, line, variety, etc., as part of an overall MAS breeding program designed to enhance yield. The present disclosure also provides chromosome QTL intervals that find equal use in MAS to select plants that demonstrate powdery mildew resistance or enhanced powdery mildew resistance. Similarly, the QTL intervals can also be used to counterselect plants that are susceptible or have reduced resistance to powdery mildew.

In some embodiments, the present disclosure provides methods for selecting a Cannabis plant with enhanced powdery mildew resistance. These methods comprise detecting a powdery mildew resistant allele at a polymorphic locus in a chromosomal segment flanked by any two of marker loci described herein. In other embodiments, these methods comprise detecting a powdery mildew resistant allele at a polymorphic locus in a chromosomal segment flanked by any two of marker loci having the sequence of genetic markers described herein. In further embodiments, these methods comprise detecting a powdery mildew resistant haplotype in a chromosomal segment flanked by any two of marker loci described herein. In other embodiments, these methods comprise detecting a powdery mildew resistant allele at a polymorphic locus in a chromosomal segment flanked by any two of marker loci described herein. In further embodiments, these methods comprise detecting a powdery mildew resistant haplotype in a chromosomal segment flanked by any two of marker loci described herein. In other embodiments, these methods comprise detecting a powdery mildew resistant allele at a polymorphic locus in a chromosomal segment flanked by any two of marker loci described herein. In further embodiments, these methods comprise detecting a powdery mildew resistant haplotype in a chromosomal segment flanked by any two of marker loci described herein.

The present disclosure also extends to a method of making a progeny Cannabis plant. The method comprises crossing a first parent Cannabis plant with a second Cannabis plant and growing the female Cannabis plant under plant growth conditions to yield Cannabis plant progeny. Methods of crossing and growing Cannabis plants are well within the ability of those of ordinary skill in the art. Such Cannabis plant progeny can be assayed for alleles associated with powdery mildew resistance and, thereby, the desired progeny selected. Such progeny plants or seed can be sold commercially, used to provide cannabis for medicinal or recreational use, processed to obtain a desired constituent of the cannabis, or further utilized in subsequent rounds of breeding. At least one of the first or second Cannabis plants is a Cannabis plant of the present disclosure in that it comprises at least one of the allelic forms of the markers of the present disclosure, such that the progeny are capable of inheriting the allele.

In some embodiments, a method of the present disclosure is applied to at least one related Cannabis plant such as from progenitor or descendant lines in the subject Cannabis plants' pedigree such that inheritance of the desired resistance allele can be traced. The number of generations separating the Cannabis plants being subject to the methods of the present disclosure will generally be from 1 to 20, commonly 1 to 5, and typically 1, 2, or 3 generations of separation, and quite often a direct descendant or parent of the Cannabis plant will be subject to the method (i.e., one generation of separation). Thus, with this technology, one skilled in the art can detect the presence or absence of powdery mildew resistance genotypes in the genomes of Cannabis plants as part of a marker assisted selection program. In one embodiment, a breeder ascertains the genotype at one or more markers for a resistant parent, which contains a powdery mildew resistance allele, and the genotype at one or more markers for a susceptible parent, which lacks the resistance allele. For example, the markers of the present disclosure can be used in MAS in crosses by subjecting the segregating progeny to MAS to maintain resistance alleles. A breeder can then reliably track the inheritance of the resistance alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the resistant parent can be reliably predicted to express the resistant phenotype; progeny that share genotypes with the susceptible parent can be reliably predicted to express the susceptible phenotype. Thus, the laborious and inefficient process of manually phenotyping the progeny for powdery mildew resistance is avoided.

By providing the positions in the Cannabis genome of the intervals and the powdery mildew resistance associated markers within, this technology also allows one skilled in the art to identify other markers within the intervals disclosed herein or linked to the chromosome intervals disclosed herein.

Closely linked markers flanking the locus of interest that have alleles in linkage disequilibrium with a resistance allele at that locus may be effectively used to select for progeny plants with enhanced powdery mildew resistance. Thus, the markers described herein, as well as other markers genetically or physically mapped to the same chromosome interval, may be used to select for Cannabis plants with enhanced powdery mildew resistance. Typically, a set of these markers will be used (e.g., 2 or more, 3 or more, 4 or more, 5 or more, 6 or more, 7 or more, 8 or more, 9 or more, 10 or more) in the flanking region above the gene and a similar set in the flanking region below the gene. Optionally, as described above, a marker within the actual gene and/or locus may also be used. The parents and their progeny may be screened for these sets of markers, and the markers that are polymorphic between the two parents may be used for selection. In an introgression program, this allows for selection of the gene or locus genotype at the more proximal polymorphic markers and selection for the recurrent parent genotype at the more distal polymorphic markers. The choice of markers actually used to practice this technology is not particularly limited and can be any marker that maps within the chromosome intervals described herein, any marker closely linked (e.g., within 20 cM) to a marker in such chromosome intervals, or any marker selected from the markers described herein. Furthermore, since there are many different types of marker detection assays known in the art, it is not intended that the type of marker detection assay (e.g. RAPDs, RFLPs, SNPs, AFLPs, etc.) used to practice this technology be limited in any way.

Additional genetic markers can be used either in conjunction with the markers described herein or independently of the markers described herein to practice the methods of the present disclosure. Publicly available marker databases from which useful markers can be obtained can also be used.

### Molecular Genetic Markers

As used herein, “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

As used herein, “marker” also refers to nucleic acid sequences complementary to the genomic sequences, such as nucleic acids used as probes. Markers corresponding to genetic polymorphisms between members of a population can be detected by methods well-established in the art. These include, for example and without limitation, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A favorable allele of a marker is the allele of the marker that co-segregates with a desired phenotype (e.g., powdery mildew resistance). As used herein, a QTL marker has a minimum of one favorable allele, although it is possible that the marker might have two or more favorable alleles found in the population. Any favorable allele of that marker can be used advantageously for the identification and construction of powdery mildew resistant plant lines. Optionally, one, two, three or more favorable allele(s) of different markers are identified in, or introgressed into a plant, and can be selected for or against during MAS. Desirably, plants or germplasm are identified that have at least one such favorable allele that positively correlates with disease tolerance or improved disease tolerance. Alternatively, a marker allele that co-segregates with powdery mildew susceptibility also finds use with the technology, since that allele can be used to identify and counter select susceptible plants. Such an allele can be used for exclusionary purposes during breeding to identify alleles that negatively correlate with powdery mildew resistance, to eliminate susceptible plants or germplasm from subsequent rounds of breeding. In the present disclosure, favorable alleles confer powdery mildew resistance. The favorable alleles conferring resistance to powdery mildew may be referred to as “resistance alleles.”

The more tightly linked a marker is with a DNA locus influencing a phenotype, the more reliable the marker is in MAS, as the likelihood of a recombination event unlinking the marker and the locus decreases. Markers containing the causal mutation for a trait, or that are within the coding sequence of a causative gene, are ideal as no recombination is expected between them and the sequence of DNA responsible for the phenotype.

Genetic markers are distinguishable from each other (as well as from the plurality of alleles of any one particular marker) on the basis of polynucleotide length and/or sequence. A number of Cannabis molecular markers are known in the art and are published or available from various sources. In general, any differentially inherited polymorphic trait (including a nucleic acid polymorphism) that segregates among progeny is a potential genetic marker.

In some embodiments of the disclosure, one or more marker alleles are selected for in a single plant or a population of plants. In these methods, plants are selected that contain favorable alleles from more than one powdery mildew resistance marker, or alternatively, favorable alleles from more than one powdery mildew resistance marker are introgressed into a desired germplasm. One of skill in the art recognizes that the identification of favorable marker alleles is germplasm-specific. The determination of which marker alleles correlate with powdery mildew resistance (or susceptibility) is determined for the particular germplasm under study. One of skill in the art recognizes that methods for identifying the favorable alleles are known in the art. Identification and use of such favorable alleles is within the scope of this disclosure. Furthermore, identification of favorable marker alleles in plant populations other than the populations used or described herein is within the scope of this disclosure.

### Marker Detection

In some aspects, methods of the disclosure utilize an amplification step to detect/genotype a marker locus, but amplification is not always a requirement for marker detection (e.g., Southern blotting and RFLP detection may be used). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

“Amplifying,” in the context of nucleic acid amplification, is any process whereby additional copies of a selected nucleic acid (or a transcribed form thereof) are produced. In some embodiments, an amplification based marker technology is used wherein a primer or amplification primer pair is admixed with genomic nucleic acid isolated from the first plant or germplasm, and wherein the primer or primer pair is complementary or partially complementary to at least a portion of the marker locus, and is capable of initiating DNA polymerization by a DNA polymerase using the plant genomic nucleic acid as a template. The primer or primer pair is extended in a DNA polymerization reaction having a DNA polymerase and a template genomic nucleic acid to generate at least one amplicon. In other embodiments, plant RNA is the template for the amplification reaction. In some embodiments, the QTL marker is an SNP type marker, and the detected allele is a SNP allele, and the method of detection is allele specific hybridization (ASH).

In general, the majority of genetic markers rely on one or more property of nucleic acids for their detection. Typical amplification methods include various polymerase-based replication methods, including the polymerase chain reaction (PCR), ligase mediated methods such as the ligase chain reaction (LCR) and RNA polymerase based amplification (e.g., by transcription) methods.

As used herein, an “amplicon” is an amplified nucleic acid, e.g., a nucleic acid that is produced by amplifying a template nucleic acid by any available amplification method (e.g., PCR, LCR, transcription, or the like).

As used herein, a “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns.

As used herein, a “template nucleic acid” is a nucleic acid that serves as a template in an amplification reaction (e.g., a polymerase-based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill in the art will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present disclosure. These probes are typically composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of other vendors.

In one embodiment, the presence or absence of a molecular marker is determined simply through nucleotide sequencing of the polymorphic marker region. This method is readily adapted to high throughput analysis as are the other methods noted above, e.g., using available high throughput sequencing methods such as sequencing by hybridization.

In alternative embodiments, in silica methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors.

While the exemplary markers provided in the figures and tables herein are SNP markers, any of the aforementioned marker types can be employed in the context of the disclosure to identify chromosome intervals encompassing genetic element(s) that contribute to superior agronomic performance (e.g., powdery mildew resistance or enhanced powdery mildew resistance).

### Primers and Probes

In general, synthetic methods for making oligonucleotides, including probes, primers, molecular beacons, PNAs (peptide nucleic acids), LNAs (locked nucleic acids), etc., are known. For example, oligonucleotides can be synthesized chemically according to a solid phase phosphoramidite triester method. Oligonucleotides, including modified oligonucleotides, can also be ordered from a variety of commercial sources.

Nucleic acid probes to the marker loci can be cloned and/or synthesized. Any suitable label can be used with a probe of the disclosure. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon.

It is not intended that the nucleic acid probes of the disclosure be limited to any particular size. In some embodiments, the molecular markers of the disclosure are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. As used in the art, PCR primers used to amplify a molecular marker are sometimes termed “PCR markers” or simply “markers.” It will be appreciated that suitable primers to be used with the technology can be designed using any suitable method. It is not intended that the technology be limited to any particular primer or primer pair. In some embodiments, the primers of the disclosure are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons. It is not intended that the primers of the disclosure be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length, or alternatively at least 500 nucleotides in length. Marker alleles in addition to those recited herein also find use with the present disclosure.

### Linkage Analysis and QTL

As used herein, “linkage” or “genetic linkage” describes the degree with which one marker locus is “associated with” another marker locus or some other locus (for example, a powdery mildew resistance locus). For example, if locus A has genes “A” or “a” and locus B has genes “B” or “b” then a cross between parent 1 with AABB and parent 2 with aabb will produce four possible gametes where the genes are segregated into AB, Ab, aB and ab. The null expectation is that there will be independent equal segregation into each of the four possible genotypes, i.e. with no linkage ¼ of the gametes will be of each genotype. Segregation of gametes into genotypes differing from ¼ is attributed to linkage. As used herein, linkage can be between two markers, or alternatively between a marker and a phenotype. A marker locus can be associated with (linked to) a trait, e.g., a marker locus can be associated with powdery mildew resistance or enhanced powdery mildew resistance when the marker locus is in linkage disequilibrium (LD) with the resistance trait. The degree of linkage of a molecular marker to a phenotypic trait (e.g., a QTL) is measured, e.g., as a statistical probability of cosegregation of that molecular marker with the phenotype.

As used herein, the linkage relationship between a molecular marker and a phenotype is given as the statistical likelihood that the particular combination of a phenotype and the presence or absence of a particular marker allele is random. Thus, the lower the probability score, the greater the likelihood that a phenotype and a particular marker will cosegregate. In some embodiments, a probability score of 0.05 (p=0.05, or a 5% probability) of random assortment is considered a significant indication of co-segregation. However, the present disclosure is not limited to this particular standard, and an acceptable probability can be any probability of less than 50% (p<0.5). For example, a significant probability can be less than 0.25, less than 0.20, less than 0.15, or less than 0.1. The phrase “closely linked,” in the present disclosure, means that recombination between two linked loci occurs with a frequency of equal to or less than about 10% (i.e., they are separated on a genetic map by not more than 10 cM). In one embodiment, any marker of the disclosure is linked (genetically and physically) to any other marker that is at or less than 50 cM distant. In another embodiment, any marker of the disclosure is closely linked (genetically and physically) to any other marker that is in close proximity, e.g., at or less than 10 cM distant. Two closely linked markers on the same chromosome can be positioned 20, 19, 18, 17, 16, 15, 14, 13, 12, 11, 10, 9, 8, 7, 6, 5, 4, 3, 2, 1, 0.75, 0.5 or 0.25 cM or less from each other.

Classical linkage analysis can be thought of as a statistical description of the relative frequencies of co-segregation of different traits. Linkage analysis is the well characterized descriptive framework of how traits are grouped together based upon the frequency with which they segregate together. That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. The further apart on a chromosome the genes reside, the less likely they are to segregate together, because homologous chromosomes recombine during meiosis. Thus, the further apart on a chromosome the genes reside, the more likely it is that there will be a crossing over event during meiosis that will result in the marker and the DNA sequence responsible for the trait the marker is designed to track segregating separately into progeny. A common measure of linkage is the frequency with which traits cosegregate. This can be expressed as a percentage of cosegregation (recombination frequency) or, also commonly, in centiMorgans (cM).

Linkage analysis is used to determine which polymorphic marker allele demonstrates a statistical likelihood of co-segregation with the powdery mildew resistance phenotype (thus, a “resistance marker allele”). Following identification of a marker allele for co-segregation with the powdery mildew resistance phenotype, it is possible to use this marker for rapid, accurate screening of plant lines for the resistance allele without the need to grow the plants through their life cycle and await phenotypic evaluations, and furthermore, permits genetic selection for the particular resistance allele even when the molecular identity of the actual resistance QTL is unknown. Tissue samples can be taken, for example, from the endosperm, embryo, or mature/developing plant and screened with the appropriate molecular marker to rapidly determine which progeny contain the desired genetics. Linked markers also remove the impact of environmental factors that can often influence phenotypic expression. Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, in the context of the present disclosure, one cM is equal to a 1% chance that a marker locus will be separated from another locus (which can be any other trait, e.g., another marker locus, or another trait locus that encodes a QTL), due to crossing over in a single generation.

When referring to the relationship between two genetic elements, such as a genetic element contributing to resistance and a proximal marker, “coupling” phase linkage indicates the state where the “favorable” allele at the resistance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for resistance) is physically linked with an “unfavorable” allele at the proximal marker locus, and the two “favorable” alleles are not inherited together (i.e., the two loci are “out of phase” with each other).

### Quantitative Trait Loci

An allele of a QTL can comprise multiple genes or other genetic factors even within a contiguous genomic region or linkage group, such as a haplotype. As used herein, an allele of a powdery mildew resistance locus can encompass more than one gene or nucleotide sequence where each individual gene or nucleotide sequence is also capable of exhibiting allelic variation and where each gene or nucleotide sequence is also capable of eliciting a phenotypic effect on the quantitative trait in question. In an aspect of the present disclosure the allele of a QTL comprises one or more genes or nucleic acid sequences that are also capable of exhibiting allelic variation. The use of the term “an allele of a QTL” is thus not intended to exclude a QTL that comprises more than one gene or other genetic factors. Specifically, an “allele of a QTL” in the present technology can denote a haplotype within a haplotype window wherein a phenotype can be powdery mildew resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. When all the alleles present at a given locus on a chromosome are the same, that plant is homozygous at that locus. If the alleles present at a given locus on a chromosome differ, that plant is heterozygous at that locus. Plants of the present disclosure may be homozygous or heterozygous at any particular resistance locus or for a particular polymorphic marker.

The principles of QTL analysis and statistical methods for calculating linkage between markers and useful QTL, or between any loci in a genome are well known in the art. Exemplary methods include penalized regression analysis, ridge regression, single point marker analysis, complex pedigree analysis, Bayesian MCMC, identity-by-descent analysis, interval mapping, composite interval mapping, and Haseman-Elston regression. QTL analyses are often performed with the help of a computer and specialized software available from a variety of public and commercial sources known to those of skill in the art.

In some embodiments of the present disclosure, a “LOD score” is used to indicate the likelihood that a marker is associated with a QTL. The LOD score essentially expresses how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arus and Moreno-Gonzalez, Plant Breeding, Hayward, Bosemark, Romagosa (eds.) Chapman & Hall, London, pp. 314-331 (1993). A log₁₀ of an odds ratio (LOD) is calculated as: LOD=log₁₀ MLE for the presence of a QTL (MLE given no linked QTL), where MLE is a maximum likelihood estimate. As used herein, a nucleic acid marker is genetically linked to a QTL, where the marker nucleic acid molecule exhibits a LOD score of greater than 2.0, as judged by interval mapping, for powdery mildew resistance or partial resistance, preferably where the marker nucleic acid molecule exhibits a LOD score of greater than 3.0, as judged by interval mapping, for powdery mildew resistance or partial resistance, more preferably where the marker nucleic acid molecule exhibits a LOD score of greater than 3.5, as judged by interval mapping, for powdery mildew resistance or partial resistance, and even more preferably where the marker nucleic acid molecule exhibits a LOD score of about 4.0, as judged by interval mapping, for powdery mildew resistance or partial resistance based on maximum likelihood methods described by Lander and Botstein, Genetics, 121:185-199 (1989), and implemented in the software package MAPMAKER/QTL (default parameters) (Lincoln and Lander, Mapping Genes Controlling Quantitative Traits Using MAPMAKER/QTL, Whitehead Institute for Biomedical Research, Massachusetts (1990)).

### Genetic Mapping

A “genetic map” is the relationship of genetic linkage among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map. A “genetic recombination frequency” is the frequency of a crossing over event (recombination) between two genetic loci. Recombination frequency can be observed by following the segregation of markers and/or traits following meiosis. A genetic recombination frequency can be expressed in centimorgans (cM). In some cases, two different markers can have the same genetic map coordinates. In that case, the two markers are in such close proximity to each other that recombination occurs between them with such low frequency that it is undetected.

Genetic maps are graphical representations of genomes (or a portion of a genome such as a single chromosome) where the distances between markers are measured by the recombination frequencies between them. Plant breeders use genetic maps of molecular markers to increase breeding efficiency through Marker assisted selection (MAS), a process where selection for a trait of interest is not based on the trait itself but rather on the genotype of a marker linked to the trait. A molecular marker that demonstrates reliable linkage with a phenotypic trait provides a useful tool for indirectly selecting the trait in a plant population, especially when accurate phenotyping is difficult, slow, or expensive.

In general, the closer two markers or genomic loci are on the genetic map, the closer they lie to one another on the physical map. A lack of precise proportionality between cM distances and physical distances can exist due to the fact that the likelihood of genetic recombination is not uniform throughout the genome; some chromosome regions are cross-over “hot spots,” while other regions demonstrate only rare recombination events, if any.

Genetic mapping variability can also be observed between different populations of the same crop species. In spite of this variability in the genetic map that may occur between populations, genetic map and marker information derived from one population generally remains useful across multiple populations in identification of plants with desired traits, counter-selection of plants with undesirable traits and in guiding MAS. As one of skill in the art will recognize, recombination frequencies (and as a result, genetic map positions) in any particular population are not static. The genetic distances separating two markers (or a marker and a QTL) can vary depending on how the map positions are determined. For example, variables such as the parental mapping populations used, the software used in the marker mapping or QTL mapping, and the parameters input by the user of the mapping software can contribute to the QTL marker genetic map relationships. However, it is not intended that the technology be limited to any particular mapping populations, use of any particular software, or any particular set of software parameters to determine linkage of a particular marker or chromosome interval with the disease tolerance phenotype. It is well within the ability of one of ordinary skill in the art to extrapolate the novel features described herein to any gene pool or population of interest, and using any particular software and software parameters. Indeed, observations regarding genetic markers and chromosome intervals in populations in addition to those described herein are readily made using the teaching of the present disclosure.

### Association Mapping

In one aspect, the present disclosure provides chromosome intervals, marker loci, germplasm for conducting genome-wide association mapping for powdery mildew resistance. Exemplary chromosome intervals and marker loci are provided herein. Smaller intervals defined by any two marker loci disclosed herein are also contemplated. Association or LD mapping techniques aim to identify genotype-phenotype associations that are significant. It is effective for fine mapping in outcrossing species where frequent recombination among heterozygotes can result in rapid LD decay. LD is non-random association of alleles in a collection of individuals, reflecting the recombinational history of that region. Thus, LD decay averages can help determine the number of markers necessary for a genome-wide association study to generate a genetic map with a desired level of resolution.

Large populations are generally better for detecting recombination, while older populations are generally associated with higher levels of polymorphism, both of which contribute to accelerated LD decay. However, smaller effective population sizes tend to show slower LD decay, which can result in more extensive haplotype conservation. Understanding of the relationships between polymorphism and recombination is useful in developing strategies for efficiently extracting information from these resources. Association analyses compare the plants' phenotypic score with the genotypes at the various loci. Subsequently, any suitable Cannabis genetic map (for example, a composite map) can be used to help observe distribution of the identified QTL markers and/or QTL marker clustering using previously determined map locations of the markers.

### Marker-Assisted Selection

“Introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a trans gene, or the like. In any case, offspring comprising the desired allele can be repeatedly backcrossed to a line having a desired genetic background and selected for the desired allele, to result in the allele becoming fixed in a selected genetic background.

A primary motivation for development of molecular markers in plant species is the potential for increased efficiency in plant breeding through marker assisted selection (MAS). Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present disclosure provides the means to identify plants that are powdery mildew resistant, exhibit enhanced powdery mildew resistance or are susceptible to powdery mildew by identifying plants having a specified allele that is linked to the phenotype or to one or more of the molecular markers described herein.

In general, MAS uses polymorphic markers that have been identified as having a significant likelihood of co-segregation with a resistance trait. Such markers are presumed to map near a gene or genes that give the plant its resistance phenotype, and are considered indicators for the desired trait, and are termed QTL markers. Plants are tested for the presence or absence of a desired allele in the QTL marker.

Identification of plants or germplasm that include a marker locus or marker loci linked to a resistance trait or traits provides a basis for performing marker assisted selection. Plants that comprise favorable markers or favorable alleles are selected for, while plants that comprise markers or alleles that are negatively correlated with tolerance can be selected against. Desired markers and/or alleles can be introgressed into plants having a desired genetic background to produce an introgressed resistance plant or germplasm. In some embodiments, it is contemplated that a plurality of resistance markers are sequentially or simultaneous selected and/or introgressed. The combinations of tolerance markers that are selected for in a single plant is not limited, and can include any combination of markers disclosed herein or any marker linked to the markers disclosed herein, or any markers located within the QTL intervals disclosed herein.

In some embodiments, the allele that is detected is a favorable allele that positively correlates with disease tolerance or improved disease tolerance. In the case where more than one marker is selected, an allele is selected for each of the markers; thus, two or more alleles are selected. In some embodiments, it can be the case that a marker locus will have more than one advantageous allele, and in that case, either allele can be selected. It will be appreciated that the ability to identify QTL marker loci alleles that correlate with powdery mildew resistance, enhanced powdery mildew resistance or susceptibility of a Cannabis plant to powdery mildew provides a method for selecting plants that have favorable marker loci as well. That is, any plant that is identified as comprising a desired marker locus (e.g., a marker allele that positively correlates with resistance) can be selected for, while plants that lack the locus, or that have a locus that negatively correlates with tolerance, can be selected against.

In some embodiments, a powdery mildew resistant first Cannabis plant or germplasm (the donor) can be crossed with a second Cannabis plant or germplasm (the recipient, depending on characteristics that are desired in the progeny) to create an introgressed Cannabis plant or germplasm as part of a breeding program designed to improve powdery mildew resistance of the recipient Cannabis plant or germplasm. In some aspects, the recipient plant can also contain one or more disease resistant loci, which can be qualitative or quantitative trait loci. In another aspect, the recipient plant can contain a transgene.

In some embodiments, the recipient Cannabis plant or germplasm will typically display increased resistance to powdery mildew conditions as compared to the first Cannabis plant or germplasm, while the introgressed Cannabis plant or germplasm will display an increased resistance to powdery mildew conditions as compared to the second plant or germplasm. An introgressed Cannabis plant or germplasm produced by these methods are also a feature of this technology.

MAS is a powerful shortcut to selecting for desired phenotypes and for introgressing desired traits into cultivars (e.g., introgressing desired traits into plant lines). MAS is easily adapted to high throughput molecular analysis methods that can quickly screen large numbers of plant or germplasm genetic material for the markers of interest and is much more cost effective than raising and observing plants for visible traits.

When a population is segregating for multiple loci affecting one or multiple traits, e.g., multiple loci involved in tolerance, or multiple loci each involved in tolerance or resistance to different diseases, the efficiency of MAS compared to phenotypic screening becomes even greater, because all of the loci can be evaluated in the lab together from a single sample of DNA.

### Marker-Assisted Backcrossing

One application of MAS is to use the resistance or enhanced resistance markers to increase the efficiency of an introgression effort aimed at introducing a resistance QTL into a desired background. If the nucleic acids from a plant are positive for a desired genetic marker allele, the plant can be self-fertilized to create a true breeding line with the same genotype, or it can be crossed with a plant with the same marker or with other characteristics to create a sexually crossed hybrid generation.

Another use of MAS in plant breeding is to assist the recovery of the recurrent parent genotype by backcross breeding. Backcross breeding is the process of crossing a progeny back to one of its parents or parent lines. Backcrossing is usually done for the purpose of introgressing one or a few loci from a donor parent (e.g., a parent comprising desirable resistance marker loci) into an otherwise desirable genetic background from the recurrent parent. The more cycles of back crossing that are done, the greater the genetic contribution of the recurrent parent to the resulting introgressed variety. This is often necessary, because resistance plants may be otherwise undesirable, e.g., due to low yield, low fecundity, undesired chemotype, or the like. In contrast, strains which are the result of intensive breeding programs may have excellent yield, fecundity, favorable chemotype (e.g., THC and/or CBD levels) or the like, merely being deficient in one desired trait such as powdery mildew resistance.

Moreover, in another aspect, while maintaining the introduced markers associated with resistance, the genetic contribution of the plant providing resistance can be reduced by back-crossing or other suitable approaches. In one aspect, the nuclear genetic material derived from the donor material in the plant can be less than or about 50%, or less than or about 25%, or less than or about 13%, or less than or about 10%, or less than or about 7%, or less than or about 5%, or less than or about 3%, or less than or about 2% or less than or about 1%, as long as the recipient remains resistant to powdery mildew.

Genetic diversity is important for long term genetic gain in any breeding program. With limited diversity, genetic gain will eventually plateau when all of the favorable alleles have been fixed within the population. One objective is to incorporate diversity into a pool without losing the genetic gain that has already been made and with the minimum possible investment. MAS provides an indication of which genomic regions and which favorable alleles from the original ancestors have been selected for and conserved over time, facilitating efforts to incorporate favorable variation from other germplasm sources.

### Methods of Producing Cannabis Plants Resistant to Powdery Mildew

The present disclosure includes and provides for a method of producing Cannabis plants resistant to powdery mildew using molecular markers to select for one or more Cannabis plants containing at least one resistance loci described herein. Markers that are genetically linked to and can be used for selection of the resistance loci described herein are described in the present disclosure.

In some embodiments, the present disclosure provides methods for creating a population of Cannabis plants that are resistant or moderately resistant to powdery mildew, which methods comprise: (a) detecting in a first population of Cannabis plants or seeds the presence of at least one marker allele that is genetically linked to and within about 20, 15, 10, 5, 2.5, 1, 0.5, or 0.25 cM of at least one resistance loci described herein; (b) selecting a Cannabis plant or seed containing the genetically linked marker allele(s); and (c) producing a population of offspring from the selected Cannabis plant or seed. In some embodiments, the detection of the marker allele genetically linked to one or more powdery mildew resistance loci is performed concurrently, e.g., in a multiplexed reaction. In other embodiments, the detection of the marker allele genetically linked to one or more powdery mildew resistance loci is performed separately, e.g., in separate reaction.

In some embodiments of the disclosure, the method enables production of plants resistant to powdery mildew or having enhanced powdery mildew resistance, e.g., enhanced compared to a wild-type Cannabis plant. Systems, including automated systems for selecting plants that comprise a marker of interest, as described herein, and/or for correlating presence of the marker with powdery mildew resistance are also a feature of the disclosure. These systems can include e.g. probes relevant to marker locus detection, detectors for detecting labels on the probes, appropriate fluid handling elements and temperature controllers that mix probes and templates and/or amplify templates and systems instructions that correlate label detection to the presence of a particular marker locus or allele.

In some embodiments, this technology could be used on any plant selected from the genus Cannabis. In one embodiment, the plant is selected from the species Cannabis sativa.

In some embodiments, the present disclosure provides a Cannabis plant to be assayed for resistance or susceptibility to powdery mildew by any method to determine whether a plant is resistant, susceptible, or whether it exhibits some degree of resistance or susceptibility. Populations of plants can be similarly characterized in this manner, or further characterized as segregating for the trait of powdery mildew resistance.

It is further understood that a Cannabis plant of the present disclosure may exhibit the characteristics of any chemotype. As used herein, the term “chemotype” refers to the cannabinoid chemical phenotype in individual Cannabis strains. In general, chemotype is primarily determined by, but not limited to, chemical ratios or predominance of CBD, THC, and CBG and/or their acid counterparts CBDA, THCA, and CBGA present in mature or semi-mature Cannabis flower. For example, Small and Beckstead assigned chemotypes based on ratios of THCA and CBDA: plants producing primarily THCA (Type I), CBDA (Type III) or both THCA and CBDA (Type II) (Small & Beckstead, 1973). Much rarer CBGA-dominant hemp plants were later identified as a new chemotype (Type IV) (Fournier et al., 1987). Cannabis with less than 0.3% total THC by dry weight is recognized as hemp. In some embodiments of the present technology, the Cannabis strain has a low-THC/high-CBD chemotype, e.g., having a tetrahydrocannabinol (THC) content of between about 0.05% and about 0.5% by weight, or between about 0.05% and about 0.25% by weight, or between 0.05% and about 0.1% by weight, and/or a cannabidiol (CBD) content of between about 0.01% and about 30% by weight, or between about 0.01% and about 25% by weight, or between about 0.01% and about 20% by weight, or between about 0.01% and about 15% by weight, or between about 0.01% and about 10% by weight, or between about 1% and about 30% by weight, or between about 1% and about 25% by weight, or between about 1% and about 20% by weight, or between about 1% and about 15% by weight, or between about 1% and about 10% by weight.

The present disclosure also provides for parts of the Cannabis plants of the present disclosure. Plant parts, without limitation, include seed, endosperm, ovule and pollen. In one embodiment of the present disclosure, the plant part is a seed. In another embodiment of the present disclosure, the plant part is a plant cell.

In some embodiments, the Cannabis seed can be subjected to various treatments. For example, the seeds can be treated to improve germination by priming the seeds or by disinfection to protect against seed-born pathogens. In another embodiment, seeds can be coated with any available coating to improve, for example, plantability, seed emergence, and protection against seed-born pathogens. Seed coating can be any form of seed coating including, but not limited to, pelleting, film coating, and encrustments.

In another embodiment, the Cannabis plant can show a comparative powdery mildew resistance compared to a non-resistant control Cannabis plant. In this aspect, a control Cannabis plant will generally be genetically similar except for the powdery mildew resistance allele or alleles in question. Such plants can be grown under similar conditions with equivalent or near equivalent exposure to the powdery mildew.

In a further aspect, this disclosure provides processed products made from the disclosed Cannabis plants. Such products include, but are not limited to, meal, oil, plant extract, starch, or fermentation or digestion product.

In some embodiments, the present technology relates to an isolated nucleic acid molecule having at least about 75%, or at least about 80%, or at least about 85%, at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity to the sequence of a powdery mildew resistance marker described herein.

In some embodiments, the present technology relates to nucleic acid molecules that hybridize to the above disclosed sequences. Hybridization conditions may be stringent in that hybridization will occur if there is at least about a 96% or about a 97% sequence identity with the nucleic acid molecule described herein. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5×Denhardt's solution, 10% dextran sulfate, and 20 mg/mL denatured, sheared salmon sperm DNA, following by washing the hybridization support in 0.1×SSC at about 65° C. Other known hybridization conditions are well known and are described in Sambrook et al., Molecular Cloning: A Laboratory Manual, Third Edition, Cold Spring Harbor, N.Y. (2001).

In some embodiments, the isolated nucleic acid of the present technology comprises a nucleic acid sequence as set forth herein or a fragment thereof.

Fragments contemplated by the present technology, but are not limited to, fragments having a nucleic acid sequence as set forth in any one of the sequences described herein, as well as sequences with at least or about 85% or more sequence identity thereto.

In some embodiments, the isolated nucleic acid molecule of the present technology comprises at least and/or up to or about 15, at least and/or up to or about 20 at least and/or up to or about 25, at least and/or up to or about 30, at least and/or up to or about 40 at least and/or up to or about 50, at least and/or up to or about 60, at least and/or up to or about 70, at least and/or up to or about 80, at least and/or up to or about 90, at least and/or up to 100, at least or up to or about 200, at least or up to or about 300, at least or up or about 400, at least or up to or about 500, at least or up to or about 600, at least or up to or about 700, at least or up to or about 800, at least or up to or about 900, at least or up to or about 1000, at least or up to or about 1100, at least or up to or about 1200, at least or up to or about 1300, at least or up to or about 1400 or at least or up to or about 1500 or about 1600 contiguous nucleotides of a powdery mildew resistance marker described herein. For example, the nucleic acid molecule can be from 15 contiguous nucleotides up to 1500 contiguous nucleotides or any range or number of nucleotides there between.

The length of the nucleic acid molecule described above will depend on the intended use. For example, if the intended use is as a primer or probe, for example, for PCR amplification or for screening a library, the length of the nucleic acid molecule will be less than the full length sequence, such as a fragment of for example, about 15 to about 50 nucleotides, or at least about 15 nucleotides of the sequences described herein and/or its complement. In these embodiments, the primers or probes may be substantially identical to a highly conserved region of the nucleic acid sequence or may be substantially identical to either the 5′ or 3′ end of the DNA sequence. In some cases, these primers or probes may use universal bases in some positions so as to be ‘substantially identical’ but still provide flexibility in sequence recognition. Suitable primer and probe hybridization conditions are well known in the art.

In an embodiment, the nucleic acid molecule can be used as a primer and for example comprises the nucleic acid sequence as described herein.

In an embodiment, the nucleic acid is conjugated to and/or comprises a heterologous moiety, such as a unique tail, purification tag or detectable label. The unique tail can be a specific nucleic acid sequence. The nucleic acid can for example be end labelled (5′ or 3′) or the label can be incorporated randomly during synthesis.

In one embodiment, the present technology provides an isolated nucleic acid that encodes for the polypeptide having an amino acid sequence of a powdery mildew resistance marker described herein.

In some embodiments, the present technology relates to an isolated polypeptide having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identity to an amino acid sequence of a powdery mildew resistance marker described herein.

In some embodiments, the present technology relates to a construct or an in vitro expression system having an isolated nucleic acid molecule having at least, greater than or about 75% sequence identity to a powdery mildew resistance marker described herein. Accordingly, the present technology further relates to a method for preparing a construct or in vitro expression system including such a sequence, or a fragment thereof, for introduction of the sequence or partial sequence in a sense or anti-sense orientation, or a complement thereof, into a cell.

In some embodiments, the present technology also provides for organisms, tissues or cells such as Cannabis plants, Cannabis tissue and Cannabis cells having at least one powdery mildew resistance loci or genetic marker described herein.

In some embodiments, the present technology also provides for organisms, tissues or cells that comprise the nucleic acids and/or the polypeptides as defined herein. In some embodiments, the organisms, tissues or cells are plants, plant tissues or plant cells that exhibit enhanced powdery mildew resistance. In some instances, such plants are Cannabis plants and such plant tissues and plant cells are Cannabis tissue and Cannabis cells.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Identification of Novel Sources of Genetic Resistance to Powdery Mildew from Cannabis Strains and Development of a Segregating F1 Population

Initially, 40 commercial cannabis strains (Canopy Growth Corporation, Smiths Falls, ON, Canada) were screened to evaluate level of resistance against natural levels of powdery mildew infection. These cannabis strains were naturally inoculated by placing plants that were heavily infected with powdery mildew into the growth room and allowing the air circulation to disperse the spores. Plants were evaluated weekly starting in the 2^{nd} week after plantlets were transferred to the room using a 0-4 scale, where 0 was no visible symptoms, 1=1-10 percent, 2=11-30 percent, 3=31 to 60 percent, and 4=61 to 100 percent of total leaf area of a whole plant was infected with powdery mildew. This initial screening identified twelve strains as susceptible (score=4) and three strains with no visible PM symptoms (score=0). Results of the initial PM screenings were further confirmed in a second replicated trial using artificial inoculations.

Based on the results of the two screenings, five strains were selected as parents for the development of mapping populations. A nested association mapping (NAM) population design was used to develop an F1 mapping population with the resistant strain as a common parent and pollen accepter, and the remaining four strains as susceptible parents and pollen donors. Parental strains were treated with Silver Thiosulfate (STS) to induce male flowers to generate the above indicated crosses. Over 200 seeds from each cross were germinated to develop a 200 F1 population, and altogether 800 F1 from all four crosses were generated. Five clones from each set of 800 F1 seedlings were generated for phenotyping.

### Example 2—Phenotyping the F1 Population

Around 800 healthy F1 clones were grown using standard cannabis cultivation practices. Briefly, rooted clones were transferred to 6″×6″ rockwool blocks and grown under non-inductive vegetative growth conditions (16 hours light, 8 hours night) for three weeks. Plants were then inoculated with powdery mildew spores and grown under 12 hours light and night for inductive reproductive growth conditions. After the initial visible symptoms of powdery mildew growth were detected (around two weeks after the inoculation), plants were scored for PM symptoms using the above 0-4 scale. A total of seven weekly observations were recorded on flowering stage plants. In addition, the Area Under the Disease Progress Curve (AUDPC) over 7 weeks was calculated as a measure of PM disease severity and used for QTL analysis along with the 0-4 PM rating.

### Example 3—Whole Genome Sequencing of Parental Genomes

Genomic DNA of all five parental strains was sequenced using Illumina™ short reads sequencing technology and Pair-End (PE) and Mate-Pair (MP) and Linked-reads sequencing libraries (10× Genomics™ Chromium™). All five strains were sequenced with total depth (coverage) in the range of 190-204× (calculated based on estimated genome size of 1.6 Gbp).

The sequencing data was processed and assembled using NRGene's DeNovoMAGIC™ assembler application version 3.0. The resultant analysis generated fully assembled, unphased and fully phased genome of the five parental strains. The scaffold N50 for the genome assemblies ranged from 8.2-18.5 Mb for unphased and 0.8-3.4 Mb for the phased genome assemblies.

### Example 4—SNP Genotyping

The bi-parental F1 mapping population was genotyped using skim-based genotyping by sequencing (skim GBS) which uses a low coverage, whole genome sequencing approach for whole genome genotyping (Golicz et al., 2015). Genomic DNA from the 800 F1 was extracted using NucleoSpin™ Plant II (Macherey-Nagel™). NGS libraries were prepared using RIPTIDE™ kit (iGenomX) and sequencing was done on Illumina™ NovaSeq6000 using 150 PE sequencing. Approximately 200 progenies from each of the four populations were sequenced to around 2.1× coverage of the diploid cannabis genome size of 1.6 Gbp.

SNP genotyping and genetic maps were constructed using NRGene's GenoMAGIC™ big-data toolkit. Briefly, for SNP calling, unique k-mers were extracted from the assembled scaffolds of the parental lines of each population. The k-mers were filtered according to their coverage in the sequencing data of the parental lines and the F1 population data. These k-mers were then detected in the data of the population samples. For imputation of the missing data, a HMM-based imputation model was used to fill in the missing data for each of the progeny samples using the available skim-sequencing data of the F1 population. Further inheritance pattern in the F1 population was determined and genetic maps were generated.

### Example 5—Linkage Map Creation and QTL Identification

A QTL mapping study is performed to identify the genetic region associated with resistance/susceptibility to powdery mildew using the NRGene's GenoMAGIC™ software. The haplotype marker (SNP) segregation patterns in the nested population lines are used to construct a high resolution genetic linkage map in collaboration with NRGene, based on the Genetic Analysis of Clonal F1 and Double cross (GACD) program described by Zhang and others (2015) or the Heterozygous Mapping Strategy (HetMappS) of Hyma and others (2015). Quantitative trait locus (QTL) analyses of powdery mildew resistance/susceptibility, yield and cannabinoid content is performed using MapQTL 6 (Kyazma, Wageningen, The Netherlands) using the multiple-QTL-mapping (MQM) approach (Jansen, 1993), which tests the presence of a QTL every 1 cM between pairs of adjacent markers. A genome-wide LOD score is estimated using 1000 permutations (Doerge and Churchill, 1996) at a significance level of α=0.05. In order to control background variation caused by genetic variation in other regions of the genome and detect QTL with higher resolution, automated cofactor selection is conducted, by which a set of markers throughout the linkage map are used as cofactors. The percentage of phenotypic variation explained by QTL (% Explained) and the additive effects of loci are calculated with the maximum likelihood method (Kao, 2000). Marker patterns in the parents and individuals in the mapping population are aligned and visualized by NRGene's proprietary software (the haplotype-based marker approach was presented at PAG2018 by Dr Ruth Wagner (Monsanto), https://pag.confex.com/pag/xxvi/meetingapp.cgi/Paper/31991).

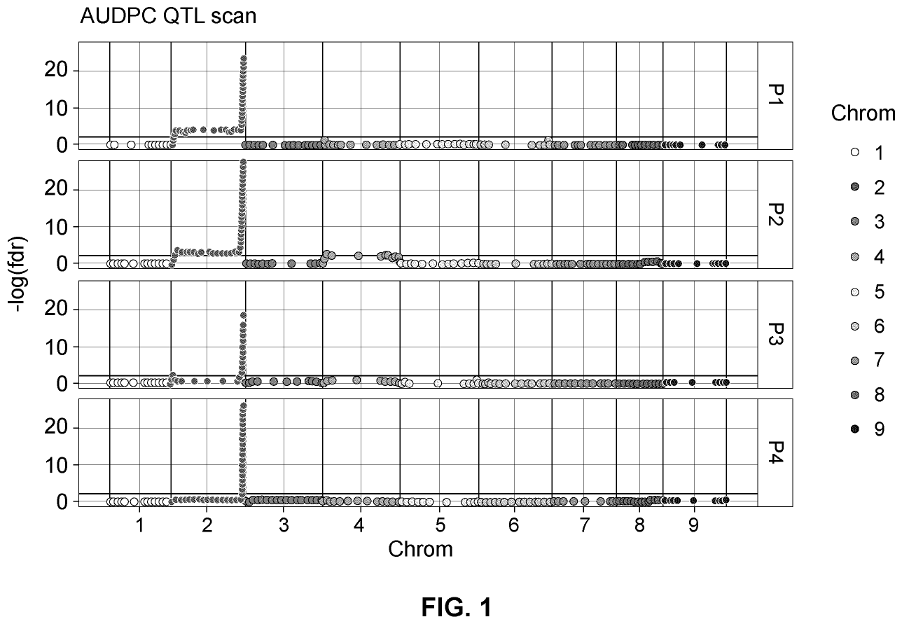

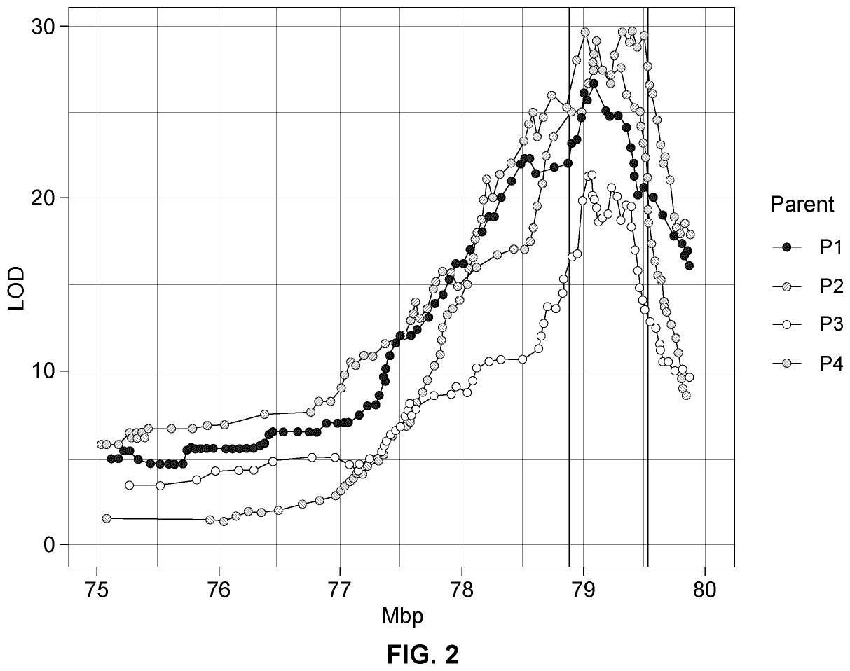

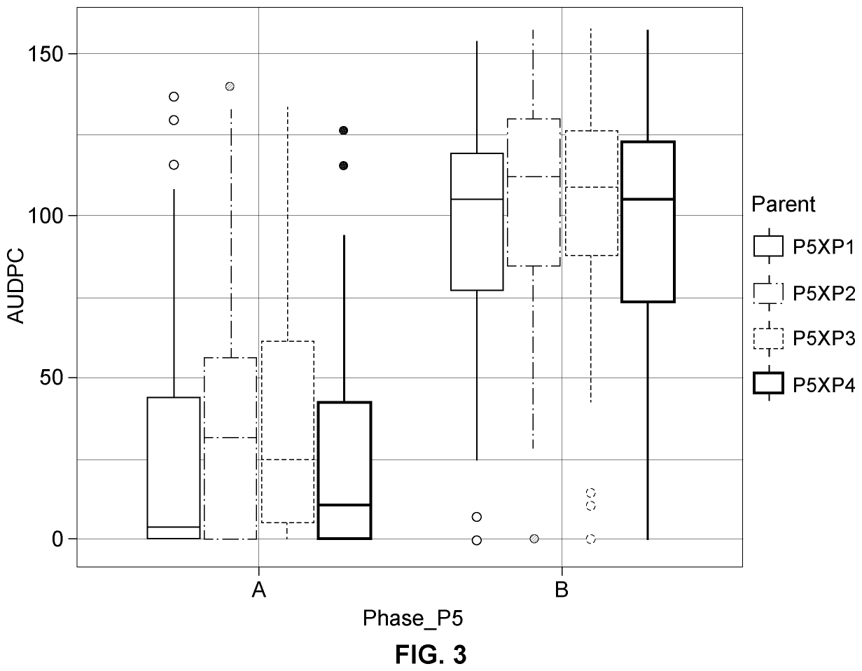

QTL analysis was conducted for 4 related pedigrees (P1, P2, P3, P4) sharing the same phenotype (powdery mildew resistance/susceptibility score). All pedigrees shared a single parent designated as P5. The phenotype (powdery mildew resistance score) was a resistance score transformed to AUDPC (Area Under the Disease Progress Curve) over 7 weeks. A genome-wide AUDPC QTL scan for each of the 4 pedigrees identified a single QTL on Chromosome 2/Phase B of P5 (78892031-79717276 bp) responsible for an increase in the disease score (which means higher susceptibility). The associated allele was inherited from the P5 parent and corresponds to genome Phase B as defined by the genome assembly. Results of the AUDPC QTL scan are shown in FIGS. 1-3, as follows: FIG. 1 shows results from the genome-wide scan for each of the 4 pedigrees; FIG. 2 shows an enlarged view of the QTL region identified on Chromosome 2; and FIG. 3 shows a chart of phenotype vs. genotype at the QTL for each of the 4 pedigrees. The coordinates of the identified QTL chromosome intervals associated with powdery mildew susceptibility (on Phase B of P5) and powdery mildew resistance (on Phase A of P5) are given in Table 1.

TABLE 1
QTL chromosome intervals associated with powdery
mildew resistance/susceptibility on Chromosome 2.

| | Associated QTL | | | |
|---|---|---|---|---|
| | chromosome | | | Size |
| Phenotype | interval | CI start | CI end | (bp) |
| Powdery mildew | On P5 Phase A | 83660977 | 84353662 | 692685 |
| resistance | | | | |
| Powdery mildew | On P5 Phase B | 78892031 | 79717276 | 825245 |
| susceptibility | | | | |

### Example 6—Gene-Based Marker Development: SNP Marker Associated with the QTL for Resistance to Cannabis Powdery Mildew

Haplotype-markers in the linkage map and physical map created by the de novo genome assembly are used to align both maps and identify candidate genes underlying the QTLs for disease resistance, yield and cannabinoid content in cannabis. Amplicon sequencing is used to confirm or identify polymorphic regions, including SNPs, in candidate genes falling into the QTL regions. Gene-specific markers are developed for the genes using a three-primer temperature switch PCR (TSP) technique (Tabone et al., 2009; Hayden et al., 2009). The assay consists of a pair of a gene (locus)-specific primers (GS) and a nested allele-specific primer (AS) that is designed to assay for SNP. Forward and reverse GS primers are designed with melting temperature between 60-65° C. (63° C. optimum), to produce s PCR product size greater than 400 bp and located at least 100 bp from the SNP. A single allele-specific primer (forward) will be designed with the melting temperature between 43-48° C. (45° C. optimum) with its 3′ end ending at the SNP.

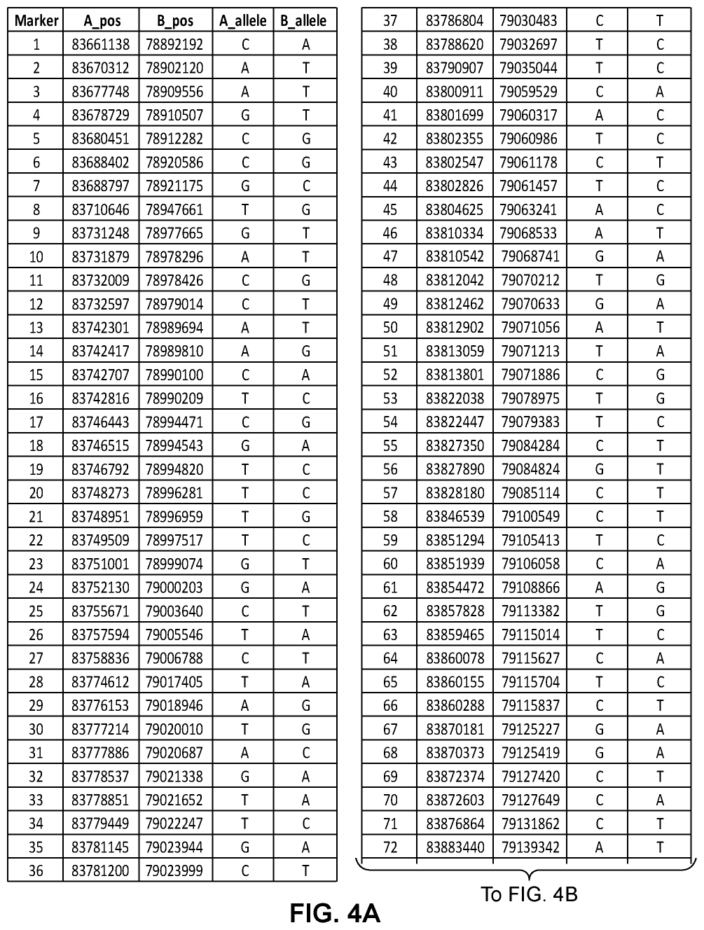

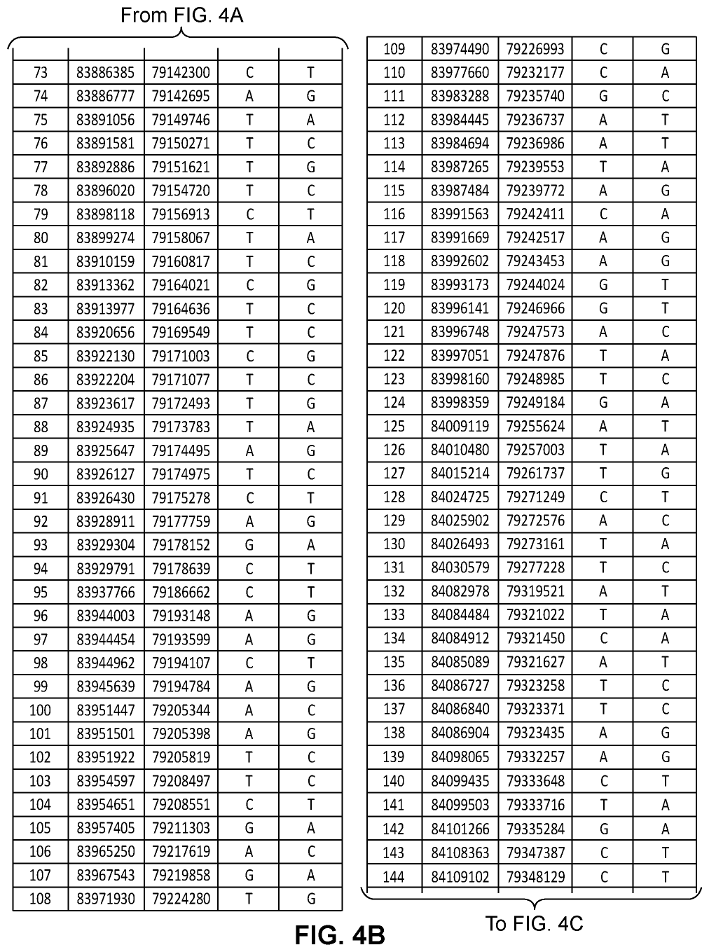

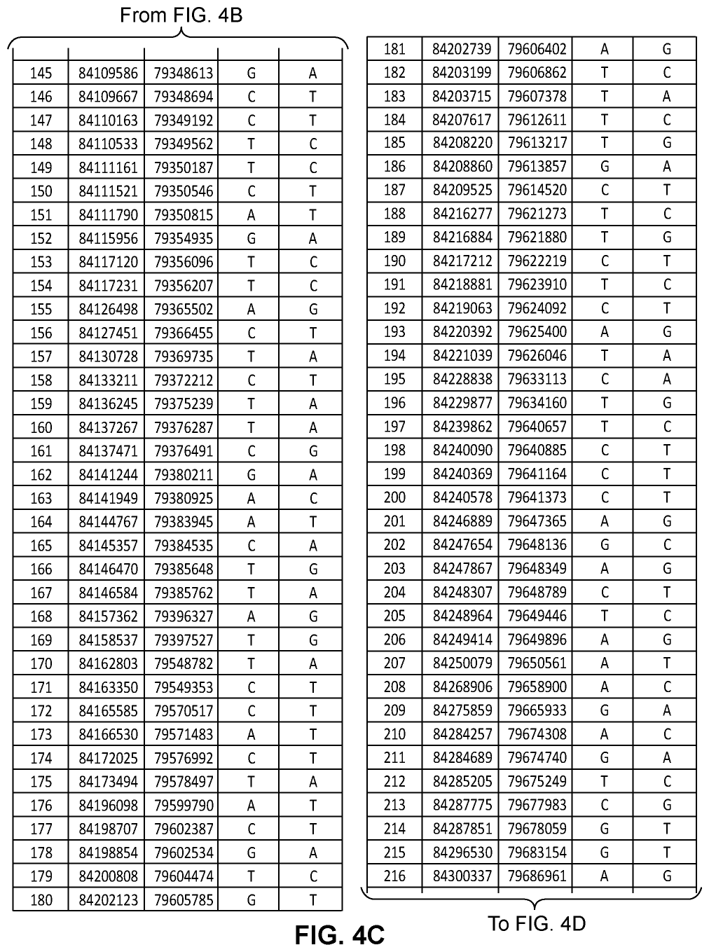

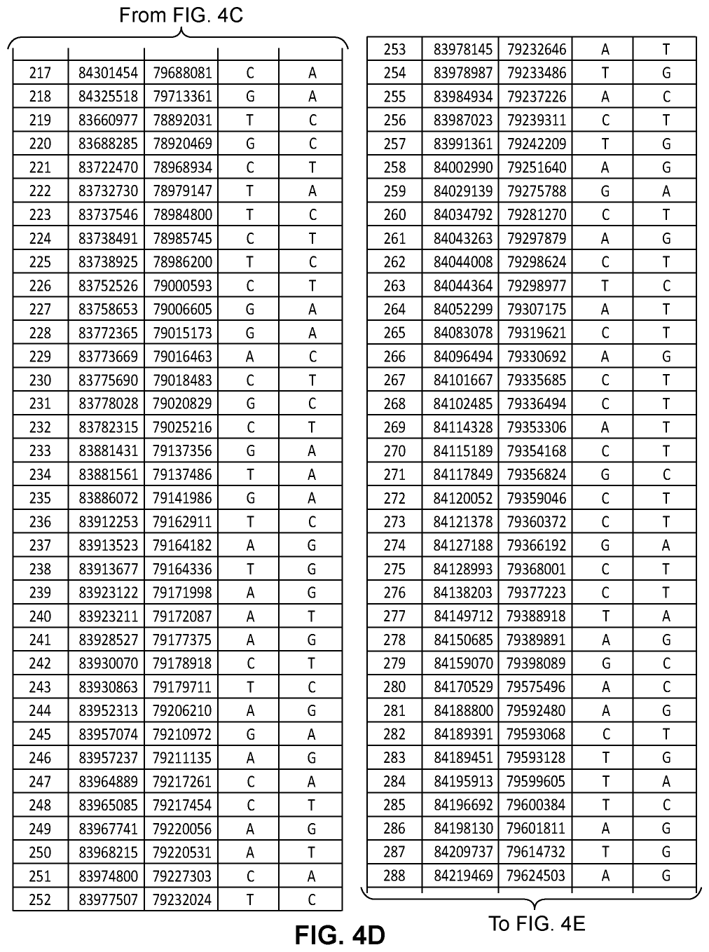

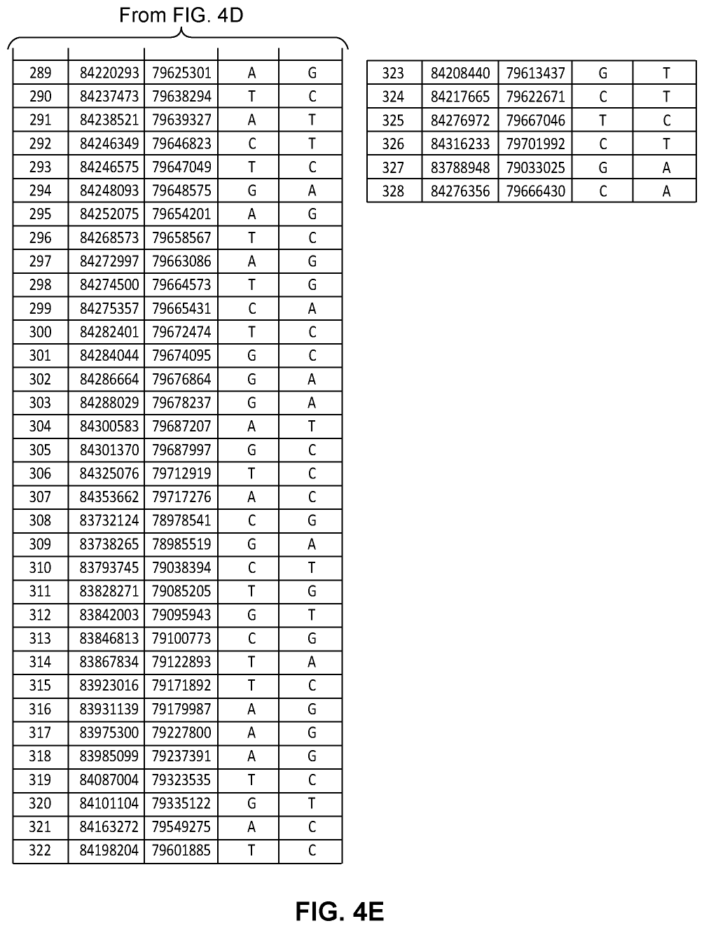

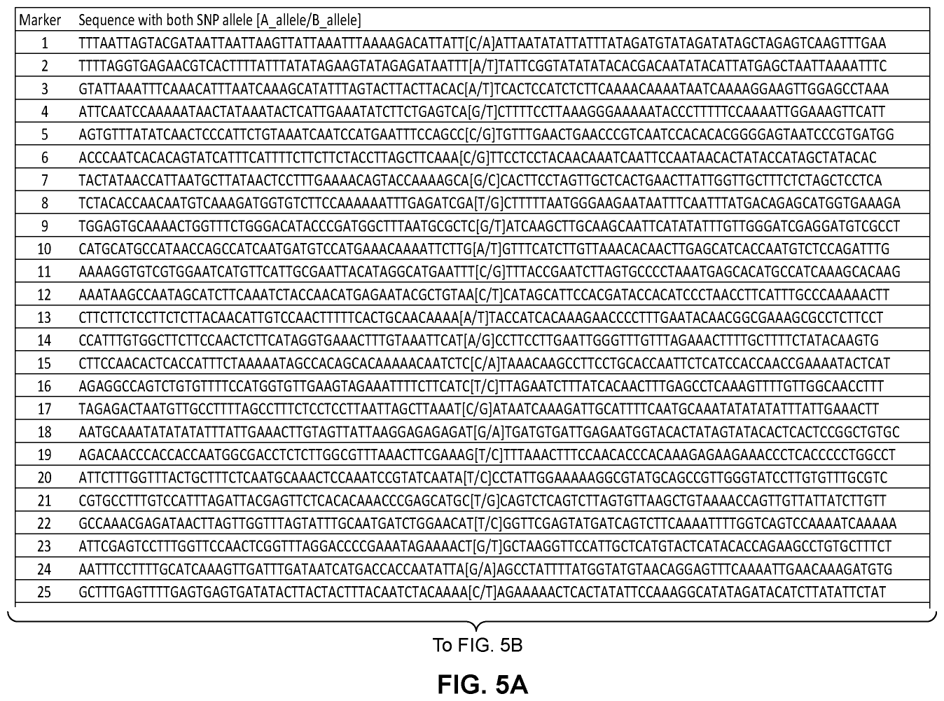

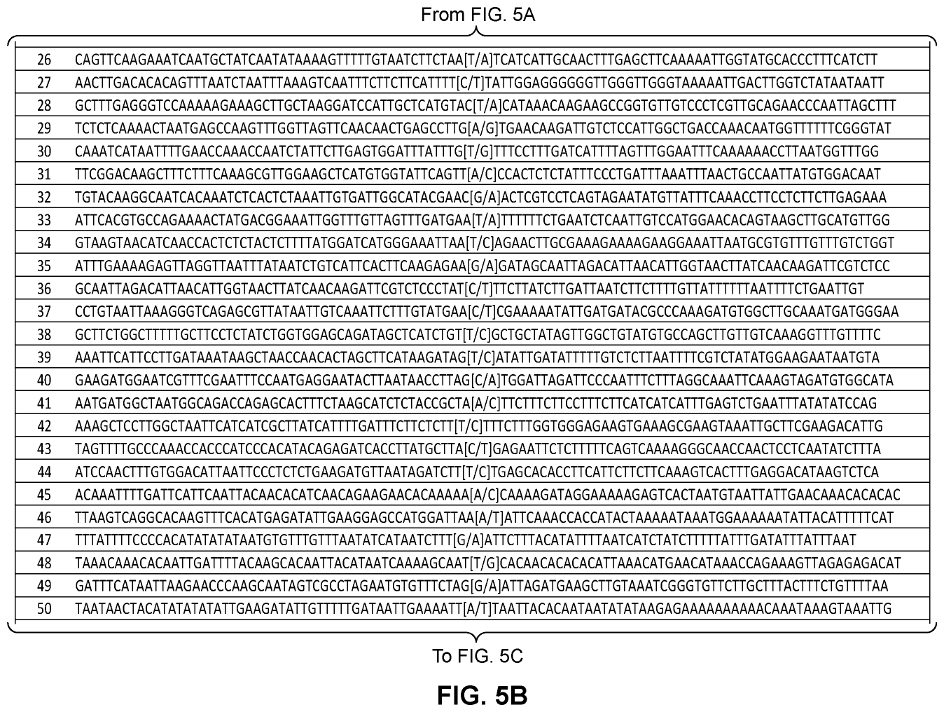

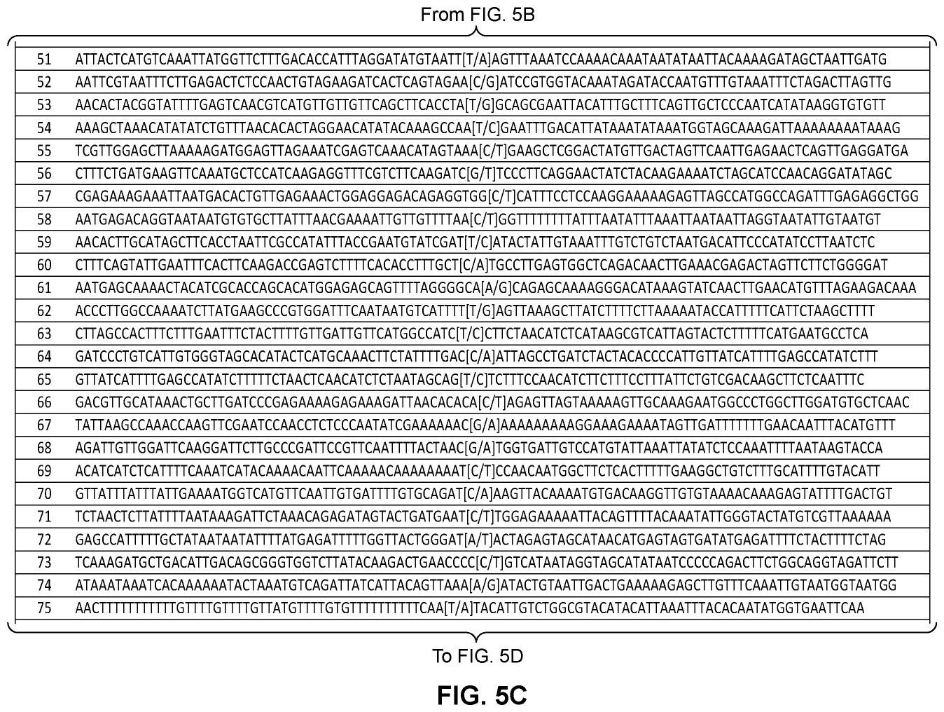

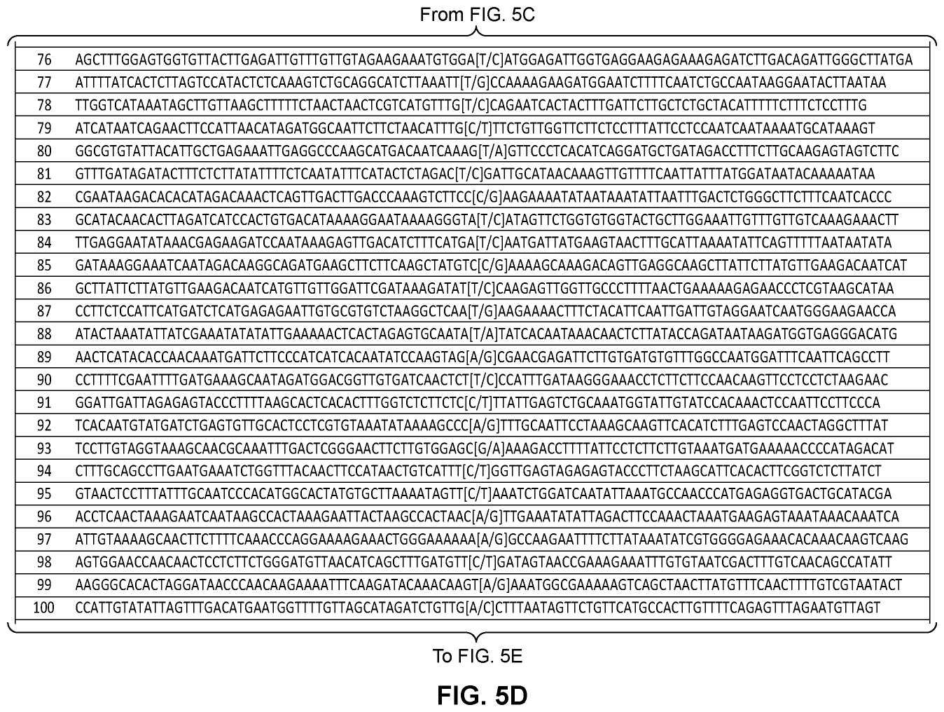

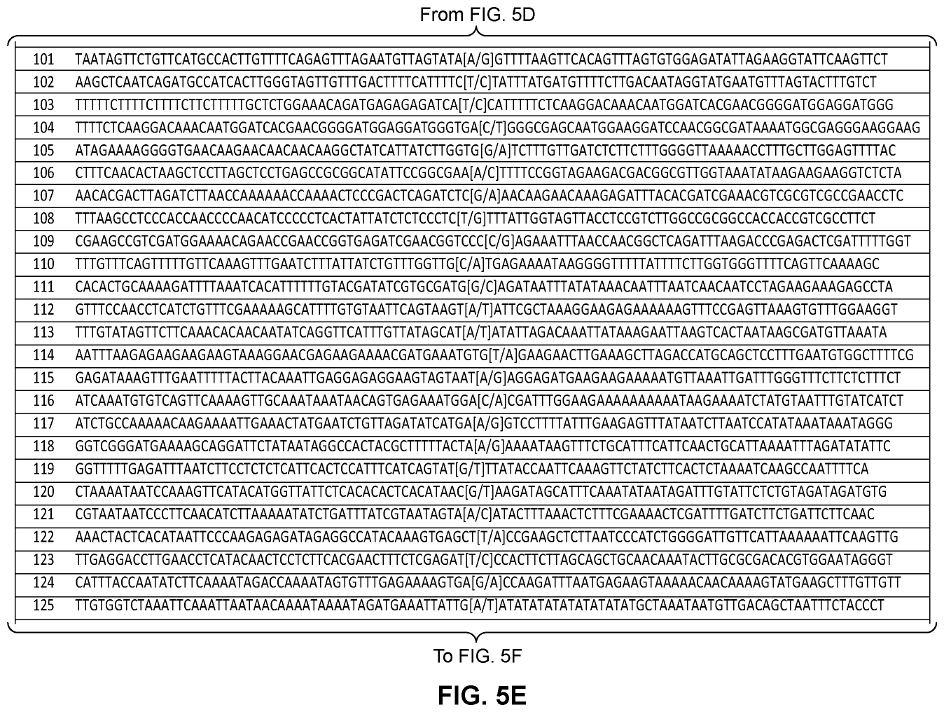

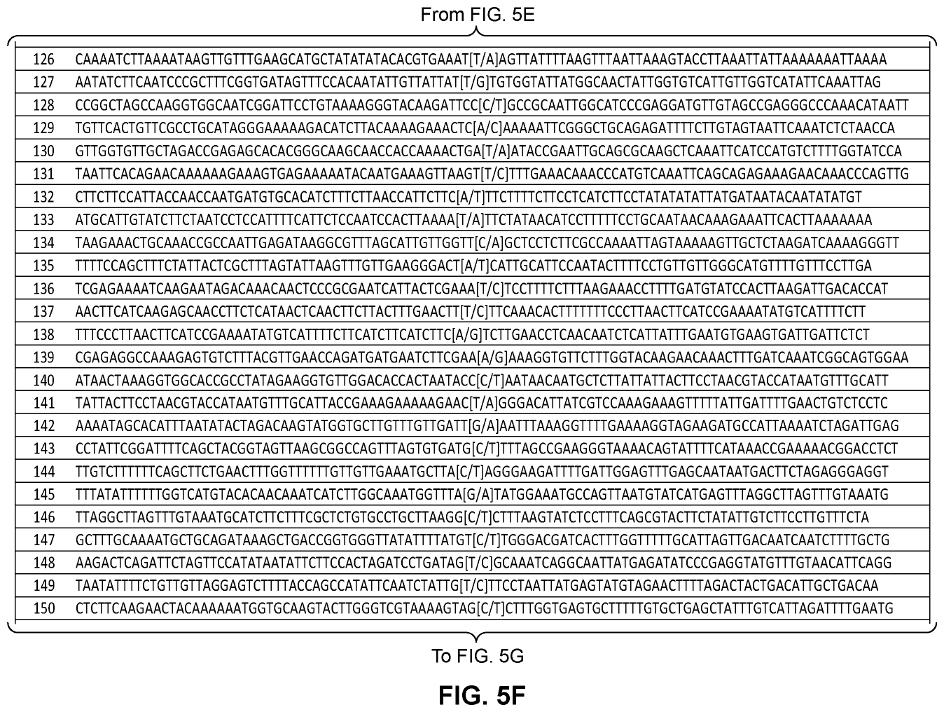

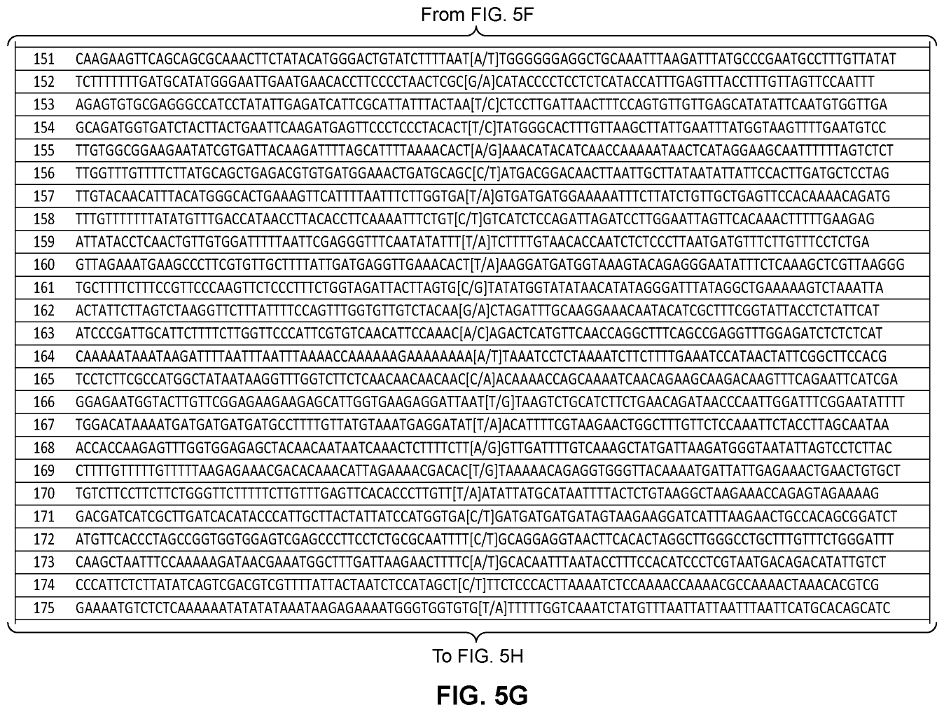

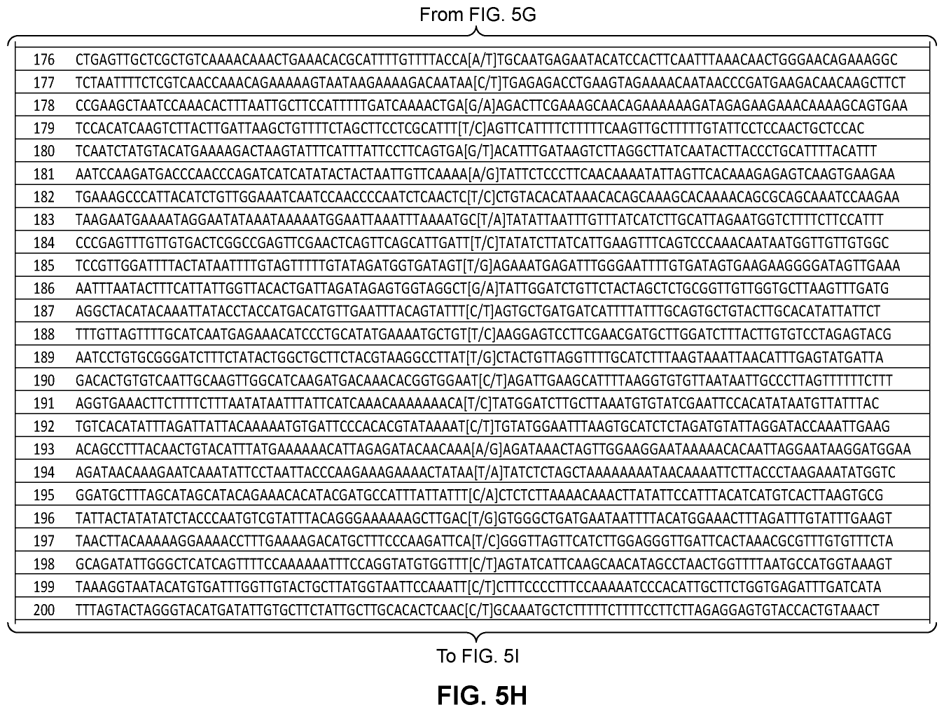

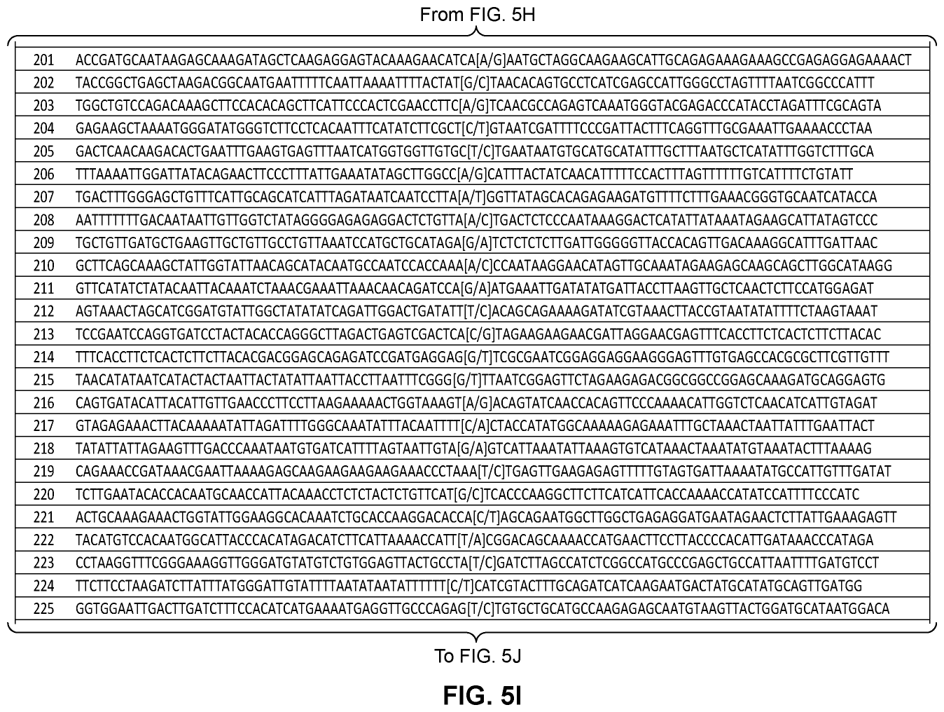

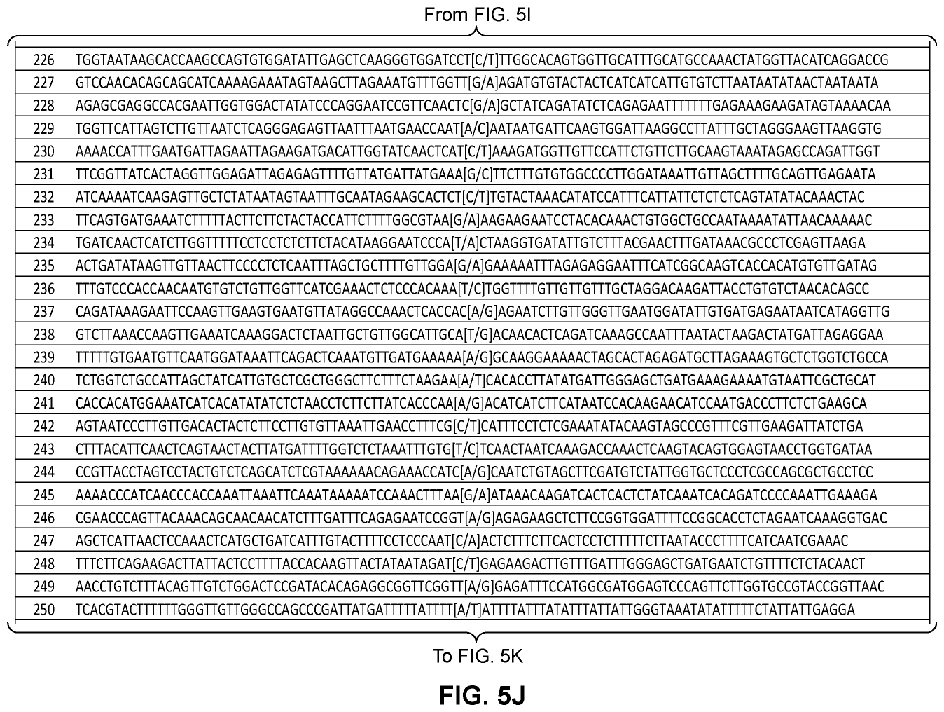

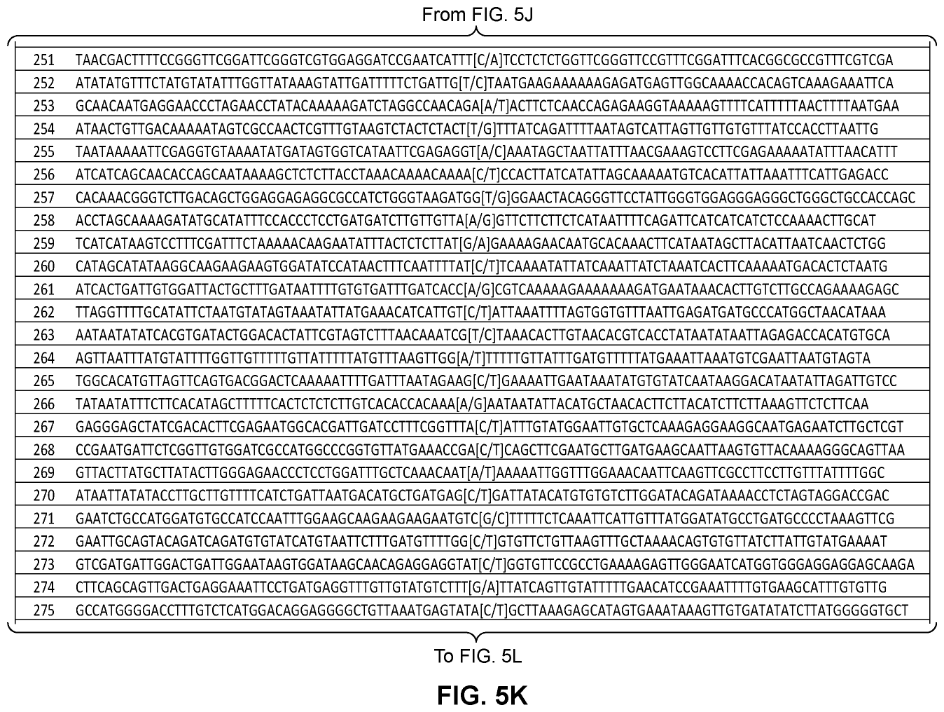

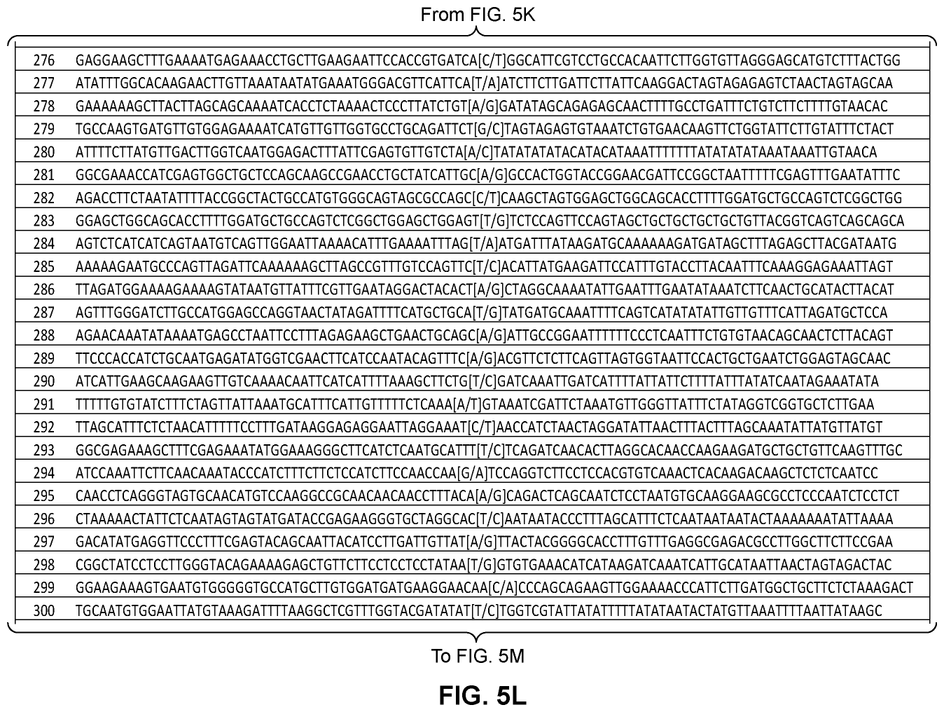

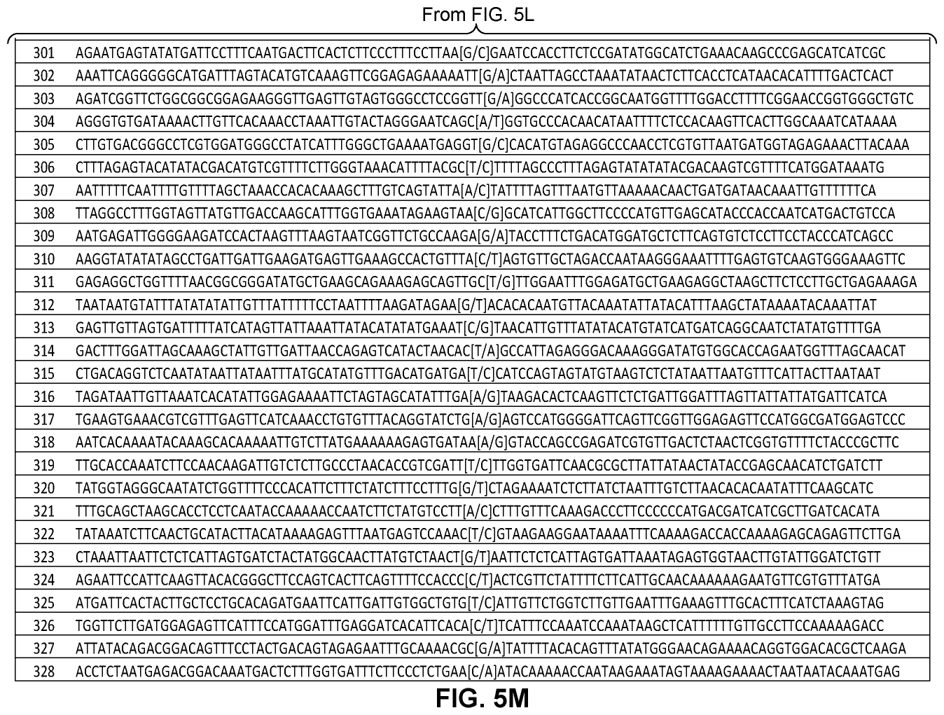

SNP markers in the QTL region on Chromosome 2 identified in Example 5 above were extracted. 328 SNP markers were identified. The SNP positions and alleles for the two phases (Phase A and Phase B) are shown in FIGS. 4A-4E. The SNP sequences are shown in FIGS. 5A-5M, in which sequences are shown in the 5′ to 3′ orientation from left to right, with the A and B alleles shown in square brackets in the center of the sequence as [A/B].

### Example 7—Validation of Powdery Mildew Linked SNP Markers and Molecular Market Development for Breeding

Molecular markers are developed to screen plants at an early stage in development for powdery mildew resistance. Validation of developed molecular markers is initiated. Marker assisted selection of resistance to powdery mildew using SNP markers is conducted.

### Example 8—Haplotype-Based Marker Development: SNP Marker Associated with the QTL for Resistance to Cannabis Powdery Mildew

### Gel Based Allele-Specific PCR Assays

To tag and select the QTL region in the F₁ generation, SNPs for marker development were selected if they are heterozygous (ab) in the maternal genome (in our cross resistant parent) and homozygous (aa) in the paternal genome (in our cross the susceptible parent). Allele-specific PCR primer were designed to genotype the SNPs located within the PM resistant QTL region on the chromosome 2. Markers in the QTL interval, and related sequences are described in table/list of sequences. Primers were designed from the SNP flanking sequences using tetra primer-amplification refractory mutation system-based polymerase chain reaction (T-ARMS-PCR; see Table 2) based approach (Medrano and Oliveira et al., 2014). Briefly, primers are designed in a such a way that two allele-specific amplicons are generated using two pairs of primers, one pair (outer forward and inner reverse) producing an amplicon representing the allele 1 and the other pair (inner forward and outer reverse) producing an amplicon representing the allele 2. The two outer primers (outer forward and outer reverse) generate the outer fragment of the SNP locus and acts as an internal PCR control. The inner PCR primer pair was designed with a deliberate mismatch at position −2 from the 3′ end to increase allele specificity of inner primers. PCR amplification was carried out at 95° C. for 2 min, followed by 35 cycles of denaturation at 95° C. for 30 see, annealing at 55-60° C. for 30 and extension at 72° C. for 1 min, and a final extension at 72° C. for 5 min. The annealing temperature was optimised for each marker separately. The resulted PCR products are visualized on 1.5% agarose gel.

TABLE 2

| | | |
|---|---|---|
| | | Genotype |
| | | pattern |
| SNP primer Id | Primer sequences | (bp) |

T-ATMS-PCR primer sequences of 91K.chr2Ap83851294 [T/C] SNP

| | | |
|---|---|---|
| 91K.chr2Ap83851294.C.IF | 5′-CCATATTTACCGAATGTATCGCTC-3′ | 302 bp |
| | (SEQ ID NO. 329) | (outer) |
| 91K.chr2Ap83851294.T.IR | 5′-TTAGACAGACAAATTTACAATAGTCTA-3′ | 203 bp |
| | (SEQ ID NO. 330) | (C allele) |
| 91K.chr2Ap83851294.OF | 5′-CTTACAATGCCACATTATTAGAGAA-3′ | 150 bp |
| | (SEQ ID NO. 331) | (T allele) |
| 91K.chr2Ap83851294.OR | 5′-ATGATACTTGGTCTGTAATCAATGA-3′ | |
| | (SEQ ID NO. 332) | |

T-ATMS-PCR primer sequences of 91K.chr2Ap83929791 [C/T] SNP

| | | |
|---|---|---|
| 91K.chr2Ap83929791.C.IF | 5′-TTTACAACTTCCATAACTGTCATGTC-3′ | 316 bp |
| | (SEQ ID NO. 333) | (outer) |
| 91K.chr2Ap83929791.T.IR | 5′-CTTAGAAGGGTACTCTCTACTCAAACA-3′ | 200 bp |
| | (SEQ ID NO. 334) | (C allele) |
| 91K.chr2Ap83929791.OF | 5′-TGTTATTCTAATGAAATCTGATCCG-3′ | 169 bp |
| | (SEQ ID NO. 335) | (T allele) |
| 91K.chr2Ap83929791.OR | 5′-GAACTTTAGTAGCAATGTGAGTTGG-3′ | |
| | (SEQ ID NO. 336) | |

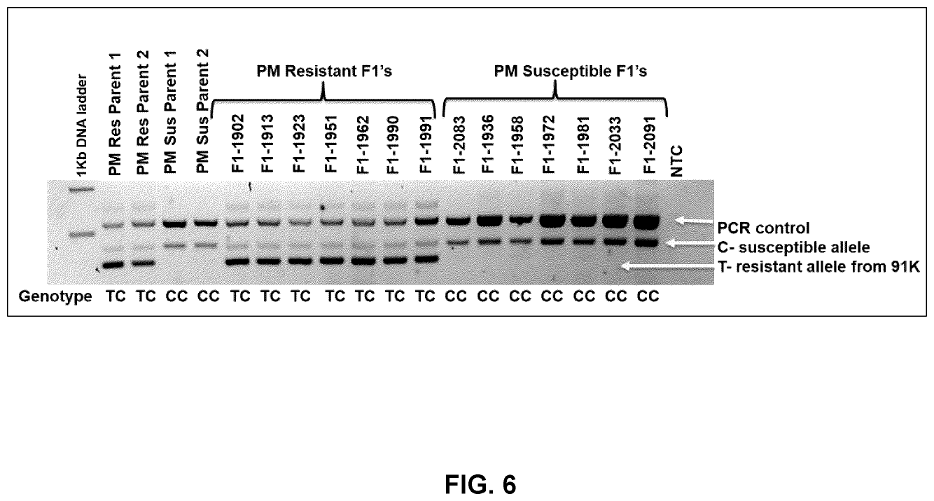

To confirm the utility of the PCR primer identifying the PM resistant and susceptible plants in the F₁ generation, known PM resistant and susceptible plants/cultivars along with the segregating F₁ progenies from the cross were genotyped using the 91K.chr2Ap83851294[T/C] and 91K.chr2Ap83929791[C/T] primers. FIG. 6 shows the results of 91K.chr2Ap83851294[T/C] SNP markers on known PM resistant and susceptible lines along with a few segregating F₁ progenies.

Prediction of disease response was made based on PCR product analysis whether or not the F₁ plant contains a heterozygous genotype similar to PM resistant parent or a homozygous genotype similar to susceptible parent. The predicted phenotypes (PM response) were confirmed with the observed response of F₁ plants in an artificially inoculated PM trial. The results are as below (Table 3). All the predicted resistant F₁ plants had the resistant genotype while the all the susceptible F₁ plants had the susceptible genotype. Table 1: Results of the PM resistant QTL linked SNP markers using agarose gel-based T-ARMS marker assays along with their predicted and observed responses in a PM inoculation trial.

TABLE 3
Confirmation of Predicted phenotype

| | | | Predicted | Observed |
|---|---|---|---|---|
| | | | phenotype | response of |
| | | | based on | plants |
| | Marker genotype for | Marker genotype for | genotype | (phenotype) in |
| | 91K.chr2Ap83851294[T/ | 91K.chr2Ap83929791[C/T] | of PM | a control PM |
| | C] [Amplicon size and | [Amplicon size and SNP | QTL SNP | inoculation |
| Plant Id | SNP genotype] | genotype] | markers | trial |
| Resistant Parent plant 1 | 302, 203 and 150 bp; TC | 316, 200 and 169 bp; CT | R | R |
| Resistant Parent plant 2 | 302, 203 and 150 bp; TC | 316, 200 and 169 bp; CT | R | R |
| Susceptible Parent plant 1 | 302 and 203 bp; CC | 316, and 169 bp; TT | S | S |
| Susceptible Parent plant 2 | 302 and 203 bp; CC | 316, and 169 bp; TT | S | S |
| F₁-1902 | 302, 203 and 150 bp; TC | 316, 200 and 169 bp; CT | R | R |
| F₁-1913 | 302, 203 and 150 bp; TC | 316, 200 and 169 bp; CT | R | R |
| F₁-1923 | 302, 203 and 150 bp; TC | 316, 200 and 169 bp; CT | R | R |
| F₁-1936 | 302 and 203 bp; CC | 316, and 169 bp; TT | S | S |
| F₁-1951 | 302, 203 and 150 bp; TC | 316, 200 and 169 bp; CT | R | R |
| F₁-1958 | 302 and 203 bp; CC | 316, and 169 bp; TT | S | S |
| F₁-1962 | 302, 203 and 150 bp; TC | 316, 200 and 169 bp; CT | R | R |
| F₁-1972 | 302 and 203 bp; CC | 316, and 169 bp; TT | S | S |
| F₁-1981 | 302 and 203 bp; CC | 316, and 169 bp; TT | S | S |
| F₁-1990 | 302, 203 and 150 bp; TC | 316, 200 and 169 bp; CT | R | R |
| F₁-1991 | 302, 203 and 150 bp; TC | 316, 200 and 169 bp; CT | R | R |
| F₁-2033 | 302 and 203 bp; CC | 316, and 169 bp; TT | S | S |
| F₁-2083 | 302 and 203 bp; CC | 316, and 169 bp; TT | S | S |
| F₁-2091 | 302 and 203 bp; CC | 316, and 169 bp; TT | S | S |

R: PM Resistant (average PM score 0-1, S: PM Susceptible (mean PM score 3-4) based on the 0-4 disease scoring scale.

### Fluorescent-Based Allele-Specific SNP Genotyping Assays

Competitive allele specific PCR for SNP genotyping assay based on dual FRET (Fluorescent Resonance Energy Transfer) including KASP (Kompetitive Allele Specific PCR (LGC, Biosearch), and/or PACE (PCR Allele Competitive Extension, (3CR Bioscience, UK) assays were developed as per the manufacturer guidelines. Briefly, for each genotyping assay, two allele-specific primers, one primer for each allele of the SNP and one common genome specific primer was designed. The allele specific primers were designed with the melting temperature between 60-67° C. (64° C. optimum) with its 3′ end ending at the SNP. The common reverse primers were designed with the melting temperature between 60-67° C. (66° C. optimum) and min and max length of 18-30 bp. The two allele-specific primers contain universal tail sequences on their 5′ ends correspond with a universal FRET cassette: one labelled with FAM™ dye and the other with HEX™ dye. The reaction master mix contains all the components required for PCR and universal FRET reporting cassettes, which binds to the corresponding tail sequences and emits fluorescence (https://www.biosearchtech.com/how-does-kasp-work). If the genotype at a given SNP is homozygous, only one of the two possible fluorescent signals will be generated, and a mixed fluorescent signal will be generated If the genotype is heterozygous. PCR reactions were optimized with both high-quality genomic DNA using commercial DNA extraction kits as well as crud DNA extracts using alkaline lysis solutions. Genotyping reactions were carried out in a volume of 10 μL (96 well plate) or 5 μL volume (384 well plate). Genotyping reactions were carried out using enzyme activation (94° C. for 15 min), 10 cycles of template denaturation at 94° C. for 20 secs and annealing and extension at 65° C. to 57° C. for 60 secs (with 0.8° C. drop per cycle), followed by 30 cycles of denaturation at 94° C. for 20 secs and annealing and extension at 57° C. for 60 secs. Finally, read the plate at 30° C. for 1 minute. Additional one or two recycling was performed with three cycles of 94° C. for 20 secs and 57° C. for 60 secs. Fluorescence detection of the reactions was performed using a QuantStudio™ 6 Flex real-time PCR system (Thermo Fisher) and the data analysed using the genotyping tool in the QuantStudio™ Design and Analysis Software Version 2.6. This generates allelic discrimination plot, with VIC or HEX as X-axis and FAM as Y-axis. PACE/KASP primer sequences linked with the QTL for PM resistance are listed in Table 4.

TABLE 4
PACE/KASP primer sequences

| PACE/KASP primer Id | Primer Sequences |
|---|---|
| 91Kv2Chr2Ap83738925-A1 | 5′-GAAGGTGACCAAGTTCATGCTT |
| | GAAAATGAGGTTGCCCAGAGT-3′ |
| | (SEQ ID NO. 337) |
| 91Kv2Chr2Ap83738925-A2 | 5′-GAAGGTCGGAGTCAACGGATTT |
| | GAAAATGAGGTTGCCCAGAGC-3′ |
| | (SEQ ID NO. 338) |
| 91Kv2Chr2Ap83738925-Com | 5′-CTCTCTTGGCATGCAGCAC |
| | A- 3′ (SEQ ID NO. 339) |
| 91Kv2Chr2Ap83851939-A1 | 5′-GAAGGTGACCAAGTTCATGCTC |
| | GAGTCTTTTCACACCTTTGCTC-3′ |
| | (SEQ ID NO. 340) |
| 91Kv2Chr2Ap83851939-A2 | 5′-GAAGGTCGGAGTCAACGGATTC |
| | GAGTCTTTTCACACCTTTGCTA-3′ |
| | (SEQ ID NO. 341) |
| 91Kv2Chr2Ap83851939-Com | 5′-TTGTCTGAGCCACTCAAGGC |
| | A-3′ (SEQ ID NO. 342) |
| 91kv2Chr2Ap84141949-A1 | 5′-GAAGGTGACCAAGTTCATGCTC |
| | CATTCGTGTCAACATTCCAAAC |
| | A- 3′ (SEQ ID NO. 343) |
| 91kv2Chr2Ap84141949-A2 | 5′-GAAGGTCGGAGTCAACGGATTC |
| | CATTCGTGTCAACATTCCAAAC |
| | C-3′ (SEQ ID NO. 344) |
| 91kv2Chr2Ap84141949-Com | 5′-GAAAGCCTGGTTGAACATGAGT |
| | CT- 3′ (SEQ ID NO. 345) |
| 91kv2Chr2Ap84085089-A1 | 5′-GAAGGTGACCAAGTTCATGCTA |
| | CAGGAAAAGTATTGGAATGCAATG |
| | T-3′ (SEQ ID NO. 346) |
| 91kv2Chr2Ap84085089-A2 | 5′-GAAGGTCGGAGTCAACGGATTA |
| | CAGGAAAAGTATTGGAATGCAATG |
| | A-3′ (SEQ ID NO. 347) |
| 91kv2Chr2Ap84085089-Com | 5′-GCTTTAGTATTAAGTTTGTTGA |
| | AGGGACT-3′ |
| | (SEQ ID NO. 348) |
| 91kv2Chr2Ap84026493-A1 | 5′-GAAGGTGACCAAGTTCATGCTG |
| | CAAGCAACCACCAAAACTGAT-3′ |
| | (SEQ ID NO. 349) |
| 91kv2Chr2Ap84026493-A2 | 5′-GAAGGTCGGAGTCAACGGATTG |
| | CAAGCAACCACCAAAACTGAA-3′ |
| | (SEQ ID NO. 350) |
| 91kv2ChrAp84026493-Com1 | 5′-GCTTGCGCTGCAATTCGGTA |
| | T-3′ (SEQ ID NO. 351) |
| 91kv2Chr2Ap84275859-A1 | 5′-GAAGGTGACCAAGTTCATGCTC |
| | CTGTTAAATCCATGCTGCATAGA |
| | G-3′ (SEQ ID NO. 352) |
| 91kv2Chr2Ap84275859-A2 | 5′-GAAGGTCGGAGTCAACGGATTC |
| | CTGTTAAATCCATGCTGCATAGA |
| | A-3′ (SEQ ID NO. 353) |
| 91kv2Chr2Ap84275859-Com | 5′-GTCAACTGTGGTAACCCCC |
| | A-3′ (SEQ ID NO. 354) |

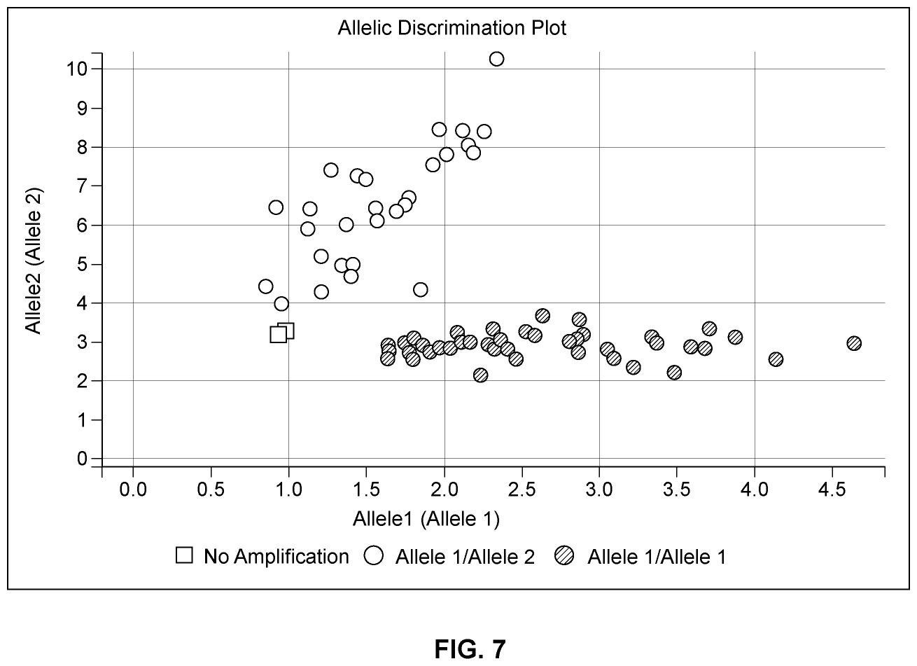

KASP/PACE SNP markers were validated on randomly selected 72 plants including parental lines and F_{P} progenies from multiple populations and standard checks. Response of these lines against the PM infection was scored in an artificially inoculated PM trial. The 25 F₁ plants with the PM resistant phenotype also had the genotype like the resistant plant (source of PM resistance QTL), while the 37 F₁ plants with the PM susceptible phenotype had the marker genotype like the susceptible parents (Table 5) (FIG. 7).

TABLE 5
Validation of PACE/KASP based SNP marker assays associated
with the QTL for resistance to cannabis powdery mildew.

| | | Observed response of F₁ plants |
|---|---|---|
| | Predicted phenotype based on | on 28 days post inoculation. |
| | genotype of KASP/PACE | Plants scoring 0-1 |
| | marker. Resistant (R) and | are considered resistant (R) |
| plant Id | Susceptible (S) | and 2-4 as susceptible (S) |
| Resistant parent | R | R |
| Susceptible parent-1 | R | R |
| Susceptible parent-2 | S | S |
| Susceptible parent 3 | S | S |
| F1-164 | R | R |
| F1-165 | S | S |
| F1-166 | S | S |
| F1-167 | R | R |
| F1-168 | R | R |
| F1-169 | S | S |
| F1-170 | S | S |
| F1-171 | S | S |
| F1-172 | R | R |
| F1-175 | S | S |
| F1-176 | S | S |
| F1-177 | R | R |
| F1-178 | S | S |
| F1-179 | S | S |
| F1-180 | R | R |
| F1-181 | R | R |
| F1-182 | S | S |
| F1-183 | S | S |
| F1-184 | S | S |
| F1-185 | S | S |
| F1-186 | S | S |
| F1-187 | S | S |
| F1-188 | S | S |
| F1-189 | R | R |
| F1-190 | R | R |
| F1-191 | R | R |
| F1-192 | S | S |
| F1-194 | R | R |
| F1-195 | S | S |
| F1-196 | S | S |
| F1-197 | R | R |
| F1-198 | R | R |
| F1-200 | R | R |
| F1-201 | S | S |
| F1-202 | S | S |
| F1-203 | R | R |
| F1-204 | R | R |
| F1-205 | S | S |
| F1-206 | S | S |
| F1-207 | S | S |
| F1-208 | R | R |
| F1-211 | S | S |
| F1-212 | S | S |
| F1-218 | S | S |
| F1-219 | S | S |
| F1-220 | R | R |
| F1-221 | S | S |
| F1-222 | S | S |
| F1-224 | R | R |
| F1-225 | R | R |
| F1-226 | S | S |
| F1-227 | R | R |
| F1-228 | S | S |
| F1-229 | R | R |
| F1-230 | R | R |
| F1-231 | S | S |
| F1-232 | R | R |
| F1-233 | S | S |
| F1-234 | R | R |
| F1-235 | S | S |
| F1-244 | S | S |
| F1-247 | S | S |
| Susceptible check 1 | S | S |
| Susceptible check 2 | S | S |

### Example 9—Validation of Powdery Mildew Linked SNP Markers and Molecular Market Development for Breeding

To validate the powdery mildew linked SNP markers and determine the effect of PM QTL in different genetic background, we developed three different F₁ populations by crossing the PM resistant cultivar Res-1 (donor of PM resistant QTL) with the three different susceptible cannabis cultivars (Sus-1, Sus-2, and Sus-3). The F₁ populations (212 plants) along with the resistant and susceptible checks were grown under standard growth conditions and artificially inculcated with PM spores as described in Example 2—“Phenotyping the F₁ population”. PM inoculated plants were scored after early visible signs of powdery mildew growth were detected (approximately two weeks after inoculation) up to four weeks post-inoculation, using a 0-4 disease scoring scale. SNP markers linked with the PM resistant QTL on chromosome 2 were used to select for the donor QTL region from 91K (PM resistant plant). For that leaf samples from the young F₁ seedlings were collected and genotyped using PM linked markers. Based on the analysis of multiple PM linked marker, F₁ plants with heterozygous allele like the resistant parent were predicted as PM resistant, while the plants with homozygous allele (like the susceptible parent) were predicted as PM susceptible. Out of 212 F₁ plants, 105 plants were predicted as PM resistant and 107 as PM susceptible plants. The predicted PM phenotype was compared with the observed response of plants scored using 0-4 disease scoring scale. All three population were analysed separately. The mean score of the predicted resistant F₁ plants per population were ranged from 0.3 to 0.7 per population, while the mean score of predicted susceptible F₁ plant per population ranged from 2.9 to 3.8 (Table 6). This high correlation between marker based predicted plant response (phenotype) with the observed plant response (phenotype) in an artificially inoculated PM trial revealed utility of the identified QTL region, sequences, and SNP markers for marker-assisted selection for powdery mildew resistance in cannabis.

TABLE 6
Results of PM marker validation on three different F₁ populations.

| | | Average observed PM | | Average observed PM |
|---|---|---|---|---|
| | Number of F₁ plants | score of predicted | Number of F₁ plants | score of predicted |
| | predicted as PM | resistant F₁ plants on | predicted as | susceptible F₁ plants |
| | resistant based on | 28 days post | susceptible based | on 28 days post |
| | the genotype of PM | inoculation using 0-4 | on the genotype of | inoculation using 0-4 |
| F₁ population | QTL markers | disease scoring scale. | PM QTL markers | disease scoring scale. |
| Res-1 X Sus-1 | 30 | 0.3 (R) | 27 | 3 (R) |
| Res-1 X Sus-2 | 36 | 0.4 (R) | 36 | 2.9 (S) |
| Res-1 X Sus-3 | 39 | 0.7 (R) | 44 | 3.8 (S) |

| | Number of | Average observed PM score |
|---|---|---|
| checks and parents | plants | based on 0-4 scale and |
| Res-1 (PM resistant parent) | 10 | 0 (R) |
| Sus-1 (PM susceptible parent) | 10 | 3.2 (S) |
| Sus-2 (PM Susceptible parent) | 10 | 3.6 (S) |
| PM Susceptible check 1) | 10 | 3.1 (S) |
| PM Susceptible check 2) | 10 | 4 (S) |

Observed PM scores of parental material and checks used in the PM screening experiment on 28 days post inoculation.
Plants scoring 0-1 disease score are considered resistant (R) and 2-4 as susceptible (S).

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### BIBLIOGRAPHY

Bengtsson, Therdse, Inger Ahman, Outi Manninen, Lars Reitan, Therese Christerson, Jens Due Jensen, Lene Krusell, Ahmed Jahoor, and Jihad Orabi. “A novel QTL for powdery mildew resistance in nordic spring barley (Hordeum vulgare L. ssp. vulgare) revealed by genome-wide association study.” Frontiers in plant science 8 (2017): 1954.
Di Marzo, V., & Piscitelli, F. (2015). The Endocannabinoid System and its Modulation by Phytocannabinoids. In Neurotherapeutics. https://doi.org/10.1007/s13311-015-0374-6
Doerge, Rebecca W., and Gary A. Churchill. “Permutation tests for multiple loci affecting a quantitative character.” Genetics 142, no. 1 (1996): 285-294.
Edwards, J. H. (1919). Haldane and the mutation rate. Journal of Genetics 8:299-309.
ElSohly, Mahmoud A., Mohamed M. Radwan, Waseem Gul, Suman Chandra, and Ahmed Galal. “Phytochemistry of Cannabis sativa L.” Phytocannabinoids (2017): 1-36.
Farnsworth, Norman R. “Pharmacognosy and Chemistry of” Cannabis Sativa”.” Journal of the American Pharmaceutical Association 9, no. 8 (1969): 410.
Fournier, G., Richez-Dumanois, C., Duvezin, J., Mathieu, J. P., & Paris, M. (1987). Identification of anew chemotype in Cannabis sativa: Cannabigerol-dominant plants, biogenetic and agronomic prospects. Planta Medica. https://doi.org/10.1055/s-2006-962705
Ge, Xintian, Weiwei Deng, Zheng Zhou Lee, Francisco J. Lopez-Ruiz, Patrick Schweizer, and Simon R. Ellwood. “Tempered mlo broad-spectrum resistance to barley powdery mildew in an Ethiopian landrace.” Scientific reports 6, no. 1 (2016): 1-10.
Golicz, A. A., P. E. Bayer, and D. Edwards. “Skim-based genotyping by sequencing In Plant Genotyping.” (2015): 257-270.
Henning, J. A., D. H. Gent, M. S. Townsend, J. L. Woods, S. T. Hill, and D. Hendrix. “QTL analysis of resistance to powdery mildew in hop (Humulus lupulus L.).” Euphytica 213, no. 4 (2017): 98.
Hyma, Katie E., Paola Barba, Minghui Wang, Jason P. Londo, Charlotte B. Acharya, Sharon E. Mitchell, Qi Sun, Bruce Reisch, and Lance Cadle-Davidson. “Heterozygous mapping strategy (HetMappS) for high resolution genotyping-by-sequencing markers: a case study in grapevine.” PloS one 10, no. 8 (2015): e0134880.
Jansen, Ritsert C. “Interval mapping of multiple quantitative trait loci.” Genetics 135, no. 1 (1993): 205-211.
Kao, Chen-Hung. “On the differences between maximum likelihood and regression interval mapping in the analysis of quantitative trait loci.” Genetics 156, no. 2 (2000): 855-865.
Li, Yahui, Xiaohan Shi, Jinghuang Hu, Peipei Wu, Dan Qiu, Yunfeng Qu, Jingzhong Xie et al. “Identification of a recessive gene PmQ conferring resistance to powdery mildew in wheat landrace Qingxinmai using BSR-Seq analysis.” Plant disease 104, no. 3 (2020): 743-751.
McPartland, J. M., & Guy, G. W. (2017). Models of Cannabis Taxonomy, Cultural Bias, and Conflicts between Scientific and Vernacular Names. Botanical Review. https://doi.org/10.1007/s12229-017-9187-0
McPartland, J. M., & Small, E. (2020). A classification of endangered high-THC Cannabis (Cannabis sativa subsp. indica) domesticates and their wild relatives. PhytoKeys. https://doi.org/10.3897/PHYTOKEYS.144.46700
Nekrasov, Vladimir, Congmao Wang, Joe Win, Christa Lanz, Detlef Weigel, and Sophien Kamoun. “Rapid generation of a transgene-free powdery mildew resistant tomato by genome deletion.” Scientific reports 7, no. 1 (2017): 1-6.
Pépin, N., Z. K. Punja, and D. L. Joly. “Occurrence of powdery mildew caused by Golovinomyces cichoracearum sensu lato on Cannabis sativa in Canada.” Plant Disease 102, no. 12 (2018): 2644-2644.
Punja, Zamir K., Danielle Collyer, Cameron Scott, Samantha Lung, Janesse Holmes, and Darren Sutton. “Pathogens and molds affecting production and quality of Cannabis sativa L.” Frontiers in plant science 10 (2019): 1120.
Ren, Yan, Weixiu Hou, Caixia Lan, Bhoja R. Basnet, Ravi P. Singh, Wei Zhu, Xiyong Cheng, Dangqun Cui, and Feng Chen. “QTL analysis and nested association mapping for adult plant resistance to powdery mildew in two bread wheat populations.” Frontiers in plant science 8 (2017): 1212.
Schultes, R. E., Schultes, R. E., Klein, W. M., Plowman, T., & Lockwood, T. E. (1974). Cannabis: an example of taxonomic neglect. Botanical Museum Leaflets, Harvard University.
Scott, Cameron, and Zamir K. Punja. “Evaluation of disease management approaches for powdery mildew on Cannabis sativa L. (marijuana) plants.” Canadian Journal of Plant Pathology (2020): 1-19.
Seifi, Alireza, Dongli Gao, Zheng Zheng, Stefano Pavan, Luigi Faino, Richard GF Visser, Anne-Marie A. Wolters, and Yuling Bai. “Genetics and molecular mechanisms of resistance to powdery mildews in tomato (Solanum lycopersicum) and its wild relatives.” European journal of plant pathology 138, no. 3 (2014): 641-665.
Small, E., & Beckstead, H. D. (1973). Cannabinoid phenotypes in Cannabis sativa. Nature. https://doi.org/10.1038/245147a0
Small, Ernest. “Interfertility and chromosomal uniformity in Cannabis.” Canadian Journal of Botany 50, no. 9 (1972): 1947-1949.
Small, Ernest, and Arthur Cronquist. “A practical and natural taxonomy for Cannabis.” Taxon (1976): 405-435.
Sun, Suli, Zhongyi Wang, Haining Fu, Canxing Duan, Xiaoming Wang, and Zhendong Zhu. “Resistance to powdery mildew in the pea cultivar Xucai 1 is conferred by the gene er1.” The Crop Journal 3, no. 6 (2015): 489-499.
Tabone, Tania, Diane F. Mather, and Matthew J. Hayden. “Temperature switch PCR (TSP): Robust assay design for reliable amplification and genotyping of SNPs.” BMC genomics 10, no. 1 (2009): 1-14.
Thompson, George Richard, J. M. Tuscano, M. Dennis, A. Singapuri, S. Libertini, R. Gaudino, A. Torres, A. M. P. Delisle, J. D. Gillece, and J. M. Schupp. “A microbiome assessment of medical marijuana.” Clin Microbiol infect 23, no. 4 (2017) 269-270.
Xu, Xuewen, Ting Yu, Ruixue Xu, Yang Shi, Xiaojian Lin, Qiang Xu, Xiaohua Qi, Yiqun Weng, and Xuehao Chen. “Fine mapping of a dominantly inherited powdery mildew resistance major-effect QTL, Pm1. 1, in cucumber identifies a 41.1 kb region containing two tandemly arrayed cysteine-rich receptor-like protein kinase genes.” Theoretical and applied genetics 129, no. 3 (2016): 507-516.
Zhang, Luyan, Lei Meng, Wencheng Wu, and Jiankang Wang. “GACD: Integrated software for genetic analysis in clonal F 1 and double cross populations.” Journal of Heredity 106, no. 6 (2015): 741-744.
Zhao, Fukai, Yinghui Li, Baoju Yang, Hongbo Yuan, Cong Jin, Lixun Zhou, Hongcui Pei et al. “Powdery mildew disease resistance and marker-assisted screening at the Pm60 locus in wild diploid wheat Triticum urartu.” The Crop Journal 8, no. 2 (2020): 252-259.
Braun, U., and Cook. R. T. A. 2012. Taxonomic Manual of the Erysiphales (Powdery Mildews). CBS Biodiversity Series 11. CBS-KNAW Fungal Biodiversity Centre, Utrecht, The Netherlands. Google Scholar
Farinas, C., and F Peduto Hand. “First Report of Golovinomyces spadiceus Causing Powdery Mildew on industrial Hemp (Cannabis sativa) in Ohio.” Plant Disease 101, no. 10 (2020): 2727-2727.
McPartland, J. M. “Cannabis pests.” Journal of the International Hemp Association 3, no. 2 (1996): 40-52.
Wiseman, Michele S, Taylor Bates, Andrea Garfinkel, Cynthia M. Ocamb, and David H. Gent. “First report of Powdery Mildew Caused by Golovinomyces ambrosiae on Cannabis sativa in Oregon.” Plant Disease ja (2021).
Medrano, Ruan Felipe Vieira, and Camila Andréa de Oliveira. “Guidelines for the tetra-primer ARMS-PCR technique development.” Molecular biotechnology 56, no. 7 (2014): 599-608.

1. A method for creating a population of Cannabis plants with enhanced powdery mildew resistance, the method comprising: a) providing a first population of Cannabis plants; b) detecting the presence of a genetic marker that is genetically linked to a powdery mildew resistance locus on a defined linkage group by 20 cM or less in the first population; c) selecting one or more Cannabis plants containing said marker from the first population of Cannabis plants; and d) producing a population of offspring from at least one of said selected Cannabis plants.

2. The method of claim 1, wherein the genetic marker detected is genetically linked to the powdery mildew resistance locus by less than about 15 cM.

3. The method of claim 1, wherein the genetic marker detected is genetically linked to the powdery mildew locus by less than about 10 cM.

4. The method of claim 1, wherein the genetic marker detected is genetically linked to the powdery mildew locus by less than about 5 cM.

5. The method of claim 1, wherein the genetic marker detected is located within a chromosome interval comprising and flanked by a first marker and a second marker.

6. The method of claim 5, wherein the chromosome interval is located on Chromosome 2.

7. The method of claim 6, wherein the chromosome interval is located on Chromosome 2 at 83660977-84353662 bp.

8. The method of claim 1, wherein the genetic marker detected is located within a chromosome interval comprising and flanked by a first nucleic acid sequence and a second nucleic acid sequence, optionally wherein the chromosome interval is located on Chromosome 2 at 83660977-84353662 bp.

9. The method of claim 1, wherein the genetic marker is selected from the markers set forth in FIGS. 4A-4E.

10. The method of claim 1, wherein the genetic marker has a sequence selected from the sequences set forth in FIGS. 5A-5M and sequences having at least, greater than or about 75%, 80%, 85%, 90%, 95%, 96%, 97%, 98% or 99% sequence identity thereto.

11. A method of creating a population of Cannabis plants comprising at least one allele associated with enhanced powdery mildew resistance comprising at least one sequence selected from a group of resistance marker nucleic acid sequences, the method comprising the steps of: a) genotyping a first population of Cannabis plants, said population containing at least one allele associated with enhanced powdery mildew resistance, the at least one allele associated with enhanced powdery mildew resistance comprising at least one sequence selected from the group consisting of resistance marker nucleic acid sequences; b) selecting from said first population one or more identified Cannabis plants containing said at least one allele associated with enhanced powdery mildew resistance comprising at least one sequence selected from the group consisting of resistance nucleic acid sequences; and c) producing from said selected Cannabis plants a second population, thereby creating a population of Cannabis plants comprising at least one allele associated with enhanced powdery mildew resistance comprising at least one sequence selected from the group consisting of resistance nucleic acid sequences.

12. The method of claim 11, wherein the at least one allele comprises at least one allele set forth in FIGS. 4A-4E.

13. The method of claim 11, wherein the at least one sequence is selected from the sequences set forth in FIGS. 5A-5M and sequences having at least, greater than or about 75%, 80%, 85%, 90%, 95%, 96%, 97%, 98% or 99% sequence identity thereto.

14. A method for creating a population of Cannabis plants with enhanced powdery mildew resistance, the method comprising: a) providing a first population of Cannabis plants; b) concurrently detecting the presence of at least one genetic marker that is genetically linked to at least one resistance loci by about 20 cM or less in the first population; c) selecting one or more Cannabis plants containing said at least one marker from the first population of Cannabis plants; and d) producing a population of offspring from at least one of said selected Cannabis plants.

15. The method of claim 14, wherein the at least one genetic marker detected is genetically linked to at least one resistance loci by less than about 15 cM.

16. The method of claim 14, wherein the at least one genetic marker detected is genetically linked to at least one resistance loci by less than about 10 cM.

17. The method of claim 14, wherein the at least one genetic marker detected is genetically linked to at least one resistance loci by less than about 5 cM.

18. The method of claim 14, wherein the at least one resistance loci is located on Chromosome 2.

19. The method of claim 18, wherein the at least one resistance loci is located within the chromosome interval on Chromosome 2 at 83660977-84353662 bp.

20. The method of claim 14, wherein the at least one genetic marker is selected from the markers set forth in FIGS. 4A-4E.

21.-56. (canceled)
